(19) United States
(12) Patent Application Publication
(10) Pub. No.: US 2026/0117214 A1
(43) Pub. Date: Apr. 30, 2026

(54) Prime Editing Systems having pegRNA with Reduced Auto-inhibitory Interaction

(71) Applicant: University of Massachusetts, Westborough, MA (US)

(72) Inventors: Scot Wolfe, Winchester, MA (US); Jonathan Watts, Worcester, MA (US); Pengpeng Liu, Worcester, MA (US); Karthikeyan Ponnienselvan, Worcester, MA (US); David Keener, Natick, MA (US); Gitali Devi, Grafton, MA (US)

(21) Appl. No.: 19/471,027

(22) PCT Filed: Feb. 16, 2024
(86) PCT No.: PCT/US2024/016216

§ 371 Date: Sep. 30, 2025

Related U.S. Application Data
(60) Provisional application No. 63/496,615, filed on Apr. 17, 2023.

(51) Int. Cl. C12N 9/22; C12N 9/12; C12N 15/11

(52) U.S. Cl. CPC C12N 9/226; C12N 9/1276; C12N 15/11; C07K 2319/80; C12N 2310/20; C12N 2310/315; C12N 2310/321; C12N 2310/322; C12N 2320/51; C12Y 207/07049

(57) ABSTRACT

Improved prime editing systems having pegRNA with reduced auto-inhibition, and methods of their use, are described.

### CROSS-REFERENCE TO RELATED APPLICATIONS

The present application claims priority to U.S. Provisional Patent Application Ser. No. 63/496,615, entitled “Prime Editing Systems having pegRNA with Reduced Auto-inhibitory Interaction” and filed Apr. 17, 2023. The foregoing application is incorporated herein by reference in its entirety.

### GOVERNMENT RIGHTS IN INVENTION

This invention was made with government support under TR002668 awarded by the National Institutes of Health. The government has certain rights in the invention.

### TECHNICAL FIELD

The present invention relates to improved prime editing systems, and more particularly to prime editing systems having improved pegRNA designs with reduced auto-inhibitory interaction.

### BACKGROUND ART

The correction of genetic mutations ex vivo or in vivo has broad potential therapeutic applications for a range of human genetic diseases. Prime editor (PE) proteins comprised of a Cas9 nickase and an engineered reverse transcriptase have enabled precise nucleotide changes, sequence insertions, and deletions. Prime editing systems do not induce double-stranded DNA breaks and do not require a donor DNA template in conjunction with homology-directed repair to introduce precise sequence changes into the genome. Unlike base editing systems, which suffer from the challenge of bystander base conversion in some sequence contexts, prime editing systems can rewrite local sequences based on a co-delivered RNA template sequence. Consequently, prime editors provide a potentially revolutionary tool for somatic genome editing, and improving the efficiency of prime editing systems would expand their therapeutic potential.

### SUMMARY OF THE EMBODIMENTS

One embodiment of the invention provides a programmable prime editing system for modification of a double-stranded target DNA sequence comprising a target strand and a complementary non-target strand, the system comprising a prime editor protein, the prime editor protein being a fusion protein comprising a nucleic acid programmable DNA binding domain fused to a reverse transcriptase domain, the DNA binding domain having nickase activity, and a pegRNA comprising, in a 5′ to 3′ direction: (i) a spacer sequence comprising a region of complementarity to the target strand of the double-stranded target DNA sequence; (ii) a gRNA core that interacts with the DNA binding domain; and (iii) an extension arm, the extension arm comprising, in a 5′ to 3′ orientation: (a) a DNA synthesis template encoding one or more nucleotide changes compared to a region downstream of a nick site in the non-target strand of the double-stranded target DNA sequence, and (b) a primer binding sequence comprising a region of complementarity to a primer sequence upstream of the nick site in the non-target strand of the double-stranded target DNA sequence and to a portion of the spacer sequence: wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.

In some embodiments, a given one of the at least one self-avoiding base and a given one of the at least one corresponding complement self-avoiding base, respectively, may be selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

In some embodiments, the pegRNA comprises a modification conferring resistance to nuclease degradation. The pegRNA may comprise at least one nucleotide comprising a modification conferring resistance to nuclease degradation. In some embodiments, a 5′ series of 1-50 nucleotides of a 5′ terminus of the pegRNA comprises a modification conferring resistance to nuclease degradation. In some embodiments, a 3′ series of 1-50 nucleotides of a 3′ terminus of the pegRNA comprises a modification conferring resistance to nuclease degradation. In some embodiments, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, the primer binding sequence consists of 5-15 nucleotides. In other embodiments, the primer binding sequence consists of 5-9 nucleotides. In some embodiments, the primer binding sequence consists of a number of nucleotides, the number of nucleotides being selected so as to provide a primer binding sequence having a melting temperature (Tm) of 34°-40° C. with the primer sequence. In other embodiments, the primer binding sequence consists of a number of nucleotides, the number of nucleotides being selected so as to provide a primer binding sequence having a melting temperature (Tm) of 35.5°-38.5° C. with the primer sequence.

The programmable prime-editing system may further comprise a competing oligonucleotide, the competing oligonucleotide being complementary to the primer binding sequence. In some embodiments, the competing oligonucleotide is further complementary to at least a portion of the DNA synthesis template. In some embodiments, the competing oligonucleotide has a length of 5-25 nucleotides. In other embodiments, the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

In some embodiments, the DNA synthesis template comprises at least one RTT self-avoiding base.

One embodiment of the invention provides a programmable prime editing system for modification of a double-stranded target DNA sequence comprising a target strand and a complementary non-target strand, the system comprising a prime editor protein, the prime editor protein being a fusion protein comprising a nucleic acid programmable DNA binding domain fused to a reverse transcriptase domain, the DNA binding domain having nickase activity, and a pegRNA comprising, in a 5′ to 3′ direction: (i) a spacer sequence comprising a region of complementarity to the target strand of the double-stranded target DNA sequence; (ii) a gRNA core that interacts with the DNA binding domain; and (iii) an extension arm, the extension arm comprising, in a 5′ to 3′ orientation: (a) a DNA synthesis template encoding one or more nucleotide changes compared to a region downstream of a nick site in the non-target strand of the double-stranded target DNA sequence, and (b) a primer binding sequence comprising a region of complementarity to a primer sequence upstream of the nick site in the non-target strand of the double-stranded target DNA sequence and to a portion of the spacer sequence: wherein a terminus of the pegRNA selected from the group consisting of a 3′ terminus, a 5′ terminus, and combinations thereof, comprises a series of 1-50 nucleotides comprising a modification conferring resistance to nuclease degradation.

In some embodiments, the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA. In some embodiments, a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

In some embodiments, independently for any given nucleotide of the series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, the primer binding sequence consists of 5-15 nucleotides. In other embodiments, the primer binding sequence consists of 5-9 nucleotides.

In some embodiments, the primer binding sequence consists of a number of nucleotides, the number of nucleotides being selected so as to provide a primer binding sequence having a melting temperature (Tm) of 34°-40° C. with the primer sequence. In other embodiments, the primer binding sequence consists of a number of nucleotides, the number of nucleotides being selected so as to provide a primer binding sequence having a melting temperature (Tm) of 35.5°-38.5° C. with the primer sequence.

In some embodiments, the programmable prime-editing system further comprises a competing oligonucleotide, the competing oligonucleotide being complementary to the primer binding sequence. In some embodiments, the competing oligonucleotide is further complementary to at least a portion of the DNA synthesis template. In some embodiments, the competing oligonucleotide has a length of 5-25 nucleotides. In some embodiments, the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

In some embodiments, the DNA synthesis template comprises at least one RTT self-avoiding base.

One embodiment of the invention provides a programmable prime editing system for modification of a double-stranded target DNA sequence comprising a target strand and a complementary non-target strand, the system comprising a prime editor protein, the prime editor protein being a fusion protein comprising a nucleic acid programmable DNA binding domain fused to a reverse transcriptase domain, the DNA binding domain having nickase activity, and a pegRNA comprising, in a 5′ to 3′ direction: (i) a spacer sequence comprising a region of complementarity to the target strand of the double-stranded target DNA sequence; (ii) a gRNA core that interacts with the DNA binding domain; and (iii) an extension arm, the extension arm comprising, in a 5′ to 3′ orientation: (a) a DNA synthesis template encoding one or more nucleotide changes compared to a region downstream of a nick site in the non-target strand of the double-stranded target DNA sequence, (b) a primer binding sequence comprising a region of complementarity to a primer sequence upstream of the nick site in the non-target strand of the double-stranded target DNA sequence and to a portion of the spacer sequence; and a competing oligonucleotide, the competing oligonucleotide being complementary to the primer binding sequence.

In some embodiments, the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA. In some embodiments, a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

In some embodiments, a 5′ series of 1-50 nucleotides of a 5′ terminus of the pegRNA comprises a modification conferring resistance to nuclease degradation. In some embodiments, a 3′ series of 1-50 nucleotides of a 3′ terminus of the pegRNA comprises a modification conferring resistance to nuclease degradation. In some embodiments, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, the primer binding sequence consists of 5-15 nucleotides. In some embodiments, the primer binding sequence consists of 5-9 nucleotides.

In some embodiments, the primer binding sequence consists of a number of nucleotides, the number of nucleotides being selected so as to provide a primer binding sequence having a melting temperature (Tm) of 34°-40° C. with the primer sequence. In some embodiments, the primer binding sequence consists of a number of nucleotides, the number of nucleotides being selected so as to provide a primer binding sequence having a melting temperature (Tm) of 355.5-38.5° C. with the primer sequence.

In some embodiments, the competing oligonucleotide is further complementary to at least a portion of the DNA synthesis template. In some embodiments, the competing oligonucleotide has a length of 5-25 nucleotides. In some embodiments, the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

In some embodiments, the DNA synthesis template comprises at least one RTT self-avoiding base.

One embodiment of the invention provides a programmable prime editing system for modification of a double-stranded target DNA sequence comprising a target strand and a complementary non-target strand, the system comprising a prime editor protein, the prime editor protein being a fusion protein comprising a nucleic acid programmable DNA binding domain fused to a reverse transcriptase domain, the DNA binding domain having nickase activity, and a pegRNA comprising, in a 5′ to 3′ direction: (i) a spacer sequence comprising a region of complementarity to the target strand of the double-stranded target DNA sequence; (ii) a gRNA core that interacts with the DNA binding domain; and (iii) an extension arm, the extension arm comprising, in a 5′ to 3′ orientation: (a) a DNA synthesis template encoding one or more nucleotide changes compared to a region downstream of a nick site in the non-target strand of the double-stranded target DNA sequence, (b) a primer binding sequence comprising a region of complementarity to a primer sequence upstream of the nick site in the non-target strand of the double-stranded target DNA sequence and to a portion of the spacer sequence, and (c) a 3′ extension, the 3′ extension being complementary to the primer binding sequence, thereby forming a hairpin with the primer binding sequence.

In some embodiments, the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA. In some embodiments, a given one of the at least one self-avoiding base and a given one of the at least one corresponding complement self-avoiding base, respectively, is selected from the group consisting of 2-aminopurine and 2-thiouracil: 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

In some embodiments, a 5′ series of 1-50 nucleotides of a 5′ terminus of the pegRNA comprises a modification conferring resistance to nuclease degradation. In some embodiments, a 3′ series of 1-50 nucleotides of a 3′ terminus of the pegRNA comprises a modification conferring resistance to nuclease degradation. In some embodiments, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, the primer binding sequence consists of 5-15 nucleotides. In some embodiments, the primer binding sequence consists of 5-9 nucleotides.

In some embodiments, the primer binding sequence consists of a number of nucleotides, the number of nucleotides being selected so as to provide a primer binding sequence having a melting temperature (Tm) of 34°-40° C. with the primer sequence. In some embodiments, the primer binding sequence consists of a number of nucleotides, the number of nucleotides being selected so as to provide a primer binding sequence having a melting temperature (Tm) of 35.5°-38.5° C. with the primer sequence.

In some embodiments, the 3′ extension is further complementary to at least a portion of the DNA synthesis template. In some embodiments, the 3′ extension is 5-25 nucleotides in length.

In some embodiments, the 3′ extension consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

In some embodiments, the DNA synthesis template comprises at least one RTT self-avoiding base.

One embodiment of the invention provides a method for site-specific modification of a double-stranded target DNA sequence comprising a target strand and a non-target strand, the method comprising contacting the double-stranded target DNA sequence with at least one of the programmable prime editing systems disclosed herein, in accordance with embodiments of the invention, wherein the contacting results in nicking the non-target strand of the double-stranded target DNA sequence to form a free 3′ end at the nick site; annealing the primer binding sequence with the primer sequence upstream of the nick site in the non-target strand of the double-stranded target DNA; synthesizing a single strand of DNA encoded by the DNA synthesis template from the free 3′ end of the non-target strand of the double-stranded target DNA sequence; and replacing the region downstream of the nick site in the non-target strand of the double-stranded target DNA sequence with the single strand of DNA encoded by the DNA synthesis template, thereby modifying the sequence of the double-stranded target DNA sequence.

One embodiment of the invention provides a non-naturally occurring pegRNA comprising, from 5′ to 3′: (i) a spacer sequence comprising a region of complementarity to a target strand of a double-stranded target DNA sequence; (ii) a gRNA core configured to interact with a DNA binding domain of a prime editor protein; and (iii) an extension arm, the extension arm comprising, in a 5′ to 3′ orientation: (a) a DNA synthesis template encoding one or more nucleotide changes compared to a region downstream of a nick site in a non-target strand of the double-stranded target DNA sequence, and (b) a primer binding sequence comprising a region of complementarity to a primer sequence upstream of the nick site in the non-target strand of the double-stranded target DNA sequence and to a portion of the spacer sequence; wherein the primer binding sequence comprises at least one self-avoiding base and the portion of the spacer sequence comprises at least one corresponding complement self-avoiding base so as to reduce base pairing of the primer binding sequence to the portion of the spacer sequence.

In some embodiments, a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

In some embodiments, a 5′ series of 1-50 nucleotides of a 5′ terminus of the pegRNA comprises a modification conferring resistance to nuclease degradation. In some embodiments, a 3′ series of 1-32 nucleotides of a 3′ terminus of the pegRNA comprises a modification conferring resistance to nuclease degradation. In some embodiments, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, the primer binding sequence consists of 5-15 nucleotides. In some embodiments, the primer binding sequence consists of 5-9 nucleotides.

In some embodiments, the primer binding sequence consists of a number of nucleotides, the number of nucleotides being selected so as to provide a primer binding sequence having a melting temperature (Tm) of 34°-40° C. with the primer sequence. In some embodiments, the primer binding sequence consists of a number of nucleotides, the number of nucleotides being selected so as to provide a primer binding sequence having a melting temperature (Tm) of 35.5°-38.5° C. with the primer sequence.

In some embodiments, the DNA synthesis template comprises at least one RTT self-avoiding base.

One embodiment of the invention provides a non-naturally occurring pegRNA comprising, from 5′ to 3′: (i) a spacer sequence comprising a region of complementarity to a target strand of a double-stranded target DNA sequence: (ii) a gRNA core configured to interact with a DNA binding domain of a prime editor protein; and (iii) an extension arm, the extension arm comprising, in a 5′ to 3′ orientation: (a) a DNA synthesis template encoding one or more nucleotide changes compared to a region downstream of a nick site in a non-target strand of the double-stranded target DNA sequence, and (b) a primer binding sequence comprising a region of complementarity to a primer sequence upstream of the nick site in the non-target strand of the double-stranded target DNA sequence and to a portion of the spacer sequence, (c) a 3′ extension, the 3′ extension being complementary to the primer binding sequence, thereby forming a hairpin with the primer binding sequence.

In some embodiments, the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA. In some embodiments, a given one of the at least one self-avoiding base and a given one of the at least one corresponding complement self-avoiding base, respectively, is selected from the group consisting of 2-aminopurine and 2-thiouracil: 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

In some embodiments, a 5′ series of 1-50 nucleotides of a 5′ terminus of the pegRNA comprises a modification conferring resistance to nuclease degradation. In some embodiments, a 3′ series of 1-32 nucleotides of a 3′ terminus of the pegRNA comprises a modification conferring resistance to nuclease degradation. In some embodiments, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, the primer binding sequence consists of 5-15 nucleotides. In some embodiments, the primer binding sequence consists of 5-9 nucleotides.

In some embodiments, the primer binding sequence consists of a number of nucleotides, the number of nucleotides being selected so as to provide a primer binding sequence having a melting temperature (Tm) of 34°-40° C. with the primer sequence. In some embodiments, the primer binding sequence consists of a number of nucleotides, the number of nucleotides being selected so as to provide a primer binding sequence having a melting temperature (Tm) of 35.5°-38.5° C. with the primer sequence.

In some embodiments, the 3′ extension is further complementary to at least a portion of the DNA synthesis template. In some embodiments, the 3′ extension is 5-25 nucleotides in length.

In some embodiments, the 3′ extension consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

In some embodiments, the DNA synthesis template comprises at least one RTT self-avoiding base.

One embodiment of the invention provides a method of treating a subject having or suspected of having a disease or disorder, the method comprising administering at least one of the programmable prime-editing systems disclosed herein, in accordance with embodiments of the invention, ex vivo, to a cell from the subject.

In some embodiments, the cell is incubated at 10°-34° C. for a period of time. In some embodiments, the cell is incubated at 32°-42° C. for the period of time. In some embodiments, the cell is incubated at 34°-40° C. for the period of time. In some embodiments, the cell is incubated at 35°-39° C. for the period of time. In some embodiments, the cell is incubated at 35.5°-38.5° C. for the period of time. In some embodiments, the cell is incubated for the period of time at a temperature, selected from the group consisting 20°±0.5° C., 21°±0.5° C., 22°±0.5° C., 23°±0.5° C., 24°±0.5° C., 25°±0.5° C., 26°±0.5° C., 27°±0.5° C., 28°±0.5° C., 29°±0.5° C., 30°±0.5° C., 31°±0.5° C., 32°±0.5° C., 33°±0.5° C., and 34°±0.5° C.

In some embodiments, period of time is 1-96 hours. In some embodiments, the period of time is 12-72 hours. In some embodiments, the period of time is 24-72 hours. In some embodiments, the period of time is 24-48 hours.

### BRIEF DESCRIPTION OF THE DRAWINGS

The foregoing features of embodiments will be more readily understood by reference to the following detailed description, taken with reference to the accompanying drawings, in which:

FIG. 1A is a bar graph showing conversion of a stop codon (TAG) to glutamine (CAG) by prime editing to restore function to a mCherry reporter in HEK293T cells using 200 ng PEmax plasmid and 100 ng pegRNA plasmid for transient transfection.

FIG. 1B is a bar graph showing conversion of a stop codon (TAG) to glutamine (CAG) by prime editing to restore function to a mCherry reporter in HEK293T cells using 1 μg PEmax mRNA and 100 pmol pegRNA for mRNA nucleofection. FIG. 1C is a bar graph showing conversion of a stop codon (TAG) to glutamine (CAG) by prime editing to restore function to a mCherry reporter in HEK293T cells using 50 pmol PEmax protein and 200 pmol pegRNA for RNP nucleofection. Frequencies of mCherry positive cells were quantified by flow cytometry 72 hours following treatment. One-way ANOVA was used to compare all the groups for each graph, PBS14 was used as a control column for multiple comparisons. ns indicates P>0.05, ** indicates P≤0.01, *** indicates P≤0.001 and **** indicates P≤0.0001. FIG. 1D is a bar graph showing PE-specified intended substitution (G·C to T·A transversion) at the +5 position of FA Complementation Group F (FANCF) site and other editing outcomes (indels and imprecise prime editing are combined) using 200 ng PEmax plasmid and 100 ng pegRNA plasmid for transient transfection. FIG. 1E is a bar graph showing PE-specified intended substitution (G·C to T·A transversion) at the +5 position of FANCF and other editing outcomes (indels and imprecise prime editing are combined) using 1 μg PEmax mRNA and 100 pmol pegRNA for mRNA nucleofection. FIG. 1F is a bar graph showing PE-specified intended substitution (G·C to T·A transversion) at the +5 position of FANCF and other editing outcomes (indels and imprecise prime editing are combined) using 50 pmol PEmax protein and 200 pmol pegRNA for RNP electroporation. Cells were harvested 72 hours following treatment. One-way ANOVA was used to compare the intended edit across all the groups for each graph, PBS13 was used as a control column for multiple comparisons. ns indicates P>0.05, ** indicates P≤0.01, and **** indicates P≤0.0001. FIG. 1G is a bar graph showing RNP-mediated PE3 editing efficiencies in an mCherry reporter cell line with different ratios of pegRNA:nk sgRNA. The amount of PEmax protein (50 μmol) and pegRNA (200 μmol) was held constant while increasing the amount of nk sgRNA delivered by electroporation. Frequency of mCherry positive cells was quantified by flow cytometry 72 hours following treatment. One-way ANOVA was used to compare all the groups for each graph, PE2 was used as a control column for multiple comparisons. ns stands for P>0.05, * indicates P≤0.05,** indicates P≤0.01, and **** indicates P≤0.0001. FIG. 1H is a bar graph showing RNP-mediated PE3 editing efficiencies at FANCF (+5 G to T) in HEK293T cells. FIG. 1I is a bar graph showing RNP-mediated PE3 editing efficiencies at HEK4 (+5 G to T) in HEK293T cells. The amount of PEmax protein (50 μmol) and pegRNA (200 μmol) was held constant while increasing the amount of nk sgRNA delivered by electroporation. Editing efficiency reflects the frequency of sequencing reads from amplicon deep sequencing that contain the intended edit or others (indels and imprecise prime editing) among all sequencing reads. Values and error bars reflect mean±s.d. of n=3 independent biological replicates. One-way ANOVA was used to compare the intended edit across all the groups for each graph, PE2 was used as a control column for multiple comparisons. ns indicates P>0.05, * indicates P≤0.05,** indicates P≤0.01, and **** indicates P≤0.0001.

FIG. 2A is a bar graph showing PE2 editing efficiencies at FANCF (+5 G to T) in HEK293T cells using unmodified pegRNAs and epegRNAs containing an evopreQ1 pseudoknot with two different PBS lengths delivered via transient transfection (200 ng PEmax-encoding plasmid with 100 ng pegRNA or epegRNA-encoding plasmid). FIG. 2B is a bar graph showing PE2 editing efficiencies in restoring function to a mCherry reporter in HEK293T cells using unmodified pegRNAs and epegRNAs containing an evopreQ1 pseudoknot with two different PBS lengths delivered via transient transfection (200 ng PEmax-encoding plasmid with 100 ng pegRNA or epegRNA-encoding plasmid). Editing efficiency reflects the frequency of sequencing reads from amplicon deep sequencing 72 hours following treatment that contain the intended edit or others (indels and imprecise prime editing) among all sequencing reads. Values and error bars reflect mean±s.d. of n=3 independent biological replicates. One-way ANOVA statistical analysis were used for FANCF precise editing and mCherry reporter editing. ns indicates P>0.05, ** indicates P≤0.01, and **** indicates P≤0.0001. FIG. 2C is a schematic of small RNA-seq library preparation. Briefly, HEK293T cells were transfected with plasmids encoding one of two effectors (SpCas9 or PEmax), and one guide RNA (sgRNA, pegRNA or epegRNA). Cells were harvested after 2 days, crosslinked, and then lysed for total RNA isolation. To sequence the bound pegRNA or epegRNA population, the SpCas9 or PEmax protein (containing 3×HA-tag) with the bound RNA were immunoprecipitated then crosslinking was reversed to purify the bound RNA. This was followed by 3′ DNA adapter ligation (3′ adapter contains 15 bp UMIs sequence) to the purified RNA, cDNA synthesis and 2 rounds of PCR to add sequencing adapters. The final library was deep sequenced and analyzed. FIG. 2D shows bulk or effector-bound RNA species present from each treatment group. Small RNAs were categorized into six species based on the length of 3′ truncation: full-length pegRNA or epegRNA, epegRNA with 3′ motif (pseudoknot) truncated, pegRNA or epegRNA with truncated but potentially functional PBS (≥7 nt remaining), pegRNA or epegRNA with truncated likely insufficient PBS (<7 nt), pegRNA or epegRNA with truncated RTT, and pegRNA or epegRNA with truncated sgRNA scaffold. Abundance of each RNA species was calculated based on UMIs incorporated into the 3′ adaptor from the small RNA-seq library.

FIG. 3A is a bar graph showing RNP-mediated PE2 editing efficiency at FANCF (+5 G to T) in HEK293T cells using varying pegRNA PBS lengths. FIG. 3B is a bar graph showing RNP-mediated PE2 editing efficiency at FANCF (+5 G to T) in different cell lines (HEK293T, U20S, RPE-1). FIG. 3C is a bar graph showing RNP-mediated PE2 editing efficiency at MECP2 (+4+5 TG to CC) in HEK293T cells using varying pegRNA PBS lengths. FIG. 3D is a bar graph showing RNP-mediated PE2 editing efficiency at HEK4 (+5 G to T) in different cell lines (HEK293T, U20S, RPE-1).

FIG. 4A is a bar graph showing RNP and mRNA-mediated PE3 editing efficiencies at FANCF (+5 G to T) in fibroblast cells at 30° C. and 37° C. FIG. 4B is a bar graph showing RNP and mRNA-mediated PE3 editing efficiencies at Mecp2 (+4+5 TG to CC) in fibroblast cells at 30° C. and 37° C. Editing efficiencies reflect the frequency of sequencing reads that contain the intended precise edit or others (indels and imprecise prime editing) among all sequencing reads. Bars and error bars represent mean±s.d. (n=3 biologically independent replicates). FIG. 4C is a bar graph showing RNP and mRNA-mediated PE3 editing efficiencies at FANCF (+5 G to T) in Primary T cells at 30° C. and 37° C. FIG. 4D is a bar graph showing RNP and mRNA-mediated PE3 editing efficiencies at CCR5 (+4+5 TG to CC) in Primary T cells at 30° C. and 37° C. Editing efficiency reflects the frequencies of sequencing reads that contain the intended precise edit and others (indels and imprecise prime editing) among all sequencing reads. Bars and error bars represent mean±s.d. (n=3 biologically independent replicates). One-way ANOVA statistical analysis was used to determine the significance of precise prime editing at different temperatures, ns indicates P>0.05, * indicates P≤0.05, and ** indicates P≤0.01.

FIG. 5A is a schematic of the architecture of the PEmax protein expression construct disclosed herein. FIG. 5A discloses SEQ ID NO: 3 (SGGSSGGSKRTAGSYPYDVPDYADGSEFESPKKKRKVSGGSSGGS) annotated as “SGGSX2-HA_Tag-SV40-SGGSX2.” FIG. 5B shows a prime editing strategy for converting the stop codon to restore mCherry expression in the reporter cell line. The asterisked sequence denotes the PAM and the underlined sequence denotes the spacer region of the pegRNA. The boxed sequence denotes the stop codon to be converted to a glutamine to restore sequence function. The nucleotides in lowercase are the edits incorporated to change the stop codon and the PAM sequence. FIG. 5B discloses SEQ ID NOS 4-5, respectively, in order of appearance. FIG. 5C shows a prime editing strategy to introduce a G→T transversion mutation at the +5 position of a FANCF target site. The asterisked sequence denotes the PAM and the underlined sequence denotes the spacer region of the pegRNA. The nucleotide lowercase denotes the edit incorporated at the +5 position. FIG. 5C discloses SEQ ID NOS 6-7, respectively, in order of appearance. FIG. 5D is a bar graph showing PE2 RNP based prime editing using pegRNA with a 14 nt PBS for mCherry in HEK293T cells. 50 pmol PEmax protein and 100 pmol pegRNA were used for electroporation of the RNP complex. Frequency of mCherry positive cells was quantified by flow cytometry. FIG. 5E is a bar graph showing PE2 RNP based prime editing using pegRNA with a 13 nt PBS for base substitution (G·C to T·A transversion) at the +5 position of FANCF site. Editing efficiency reflects the frequency of sequencing reads that contain the intended prime edit or others (indels and imprecise prime editing) among all sequencing reads from amplicon deep sequencing. Values and error bars reflect mean±s.d. of n=3 independent biological replicates.

FIG. 6A is a schematic showing that the PBS and spacer sequence within a pegRNA are complementary to each other and can potentially form intramolecular and intermolecular interactions through Watson-Crick base pairing. The complementarity can extend into the first 3 nucleotides (nt) of the RTT region if it is identical to the DNA target site. FIG. 6B is a schematic showing a DNA-competing oligonucleotide (CO) used for in vitro cleavage assays. COs complementary to the PBS or the entire PBS-RTT region were used to relieve auto-inhibitory interactions between the PBS and the spacer sequence. FIG. 6C is a gel image and a bar graph of in vitro cleavage data showing that mCherry pegRNA with a 14 nt PBS is inactive for Cas9 nuclease-based cleavage until a CO complementary to the PBS-RTT is used to disrupt the PBS< > spacer interaction. 5 pmol of Cas9 was complexed with 10 pmol of pegRNA or sgRNA and 50 pmol of CO complementary to the PBS or PBS+RTT was included where indicated. The RNP complex was incubated with 500 ng of target DNA for 20 minutes to carry out the cleavage reaction. Gel image is a representative outcome of one of three independent experiments. Values and error bars reflect mean±s.d. of n=3 independent replicates. FIG. 6D is a gel image and a bar graph of in vitro cleavage data showing that FANCF pegRNA is inactive for Cas9 nuclease based cleavage until a CO complementary to the PBS-RTT is used to disrupt the PBS< > spacer interaction. Reducing the PBS length to 7 nt results in a FANCF pegRNA that is able to program Cas9 to cleave the target site. 5 pmol of Cas9 protein was complexed with 10 pmol of pegRNA or sgRNA and 50 pmol of CO complementary to the PBS or PBS+RTT was included where indicated. The RNP complex was incubated with 500 ng of target DNA for 20 minutes to carry out the cleavage reaction. Gel image is a representative outcome of one of three independent experiments. Values and error bars reflect mean±s.d. of n=3 independent replicates. FIG. 6E is a gel image and a bar graph of in vitro cleavage data showing that reducing the length of the PBS within the mCherry pegRNA increases the Cas9 nuclease cleavage rate of a cognate target site. 5 pmol of Cas9 protein was complexed with 10 pmol of pegRNA or sgRNA. The RNP complex was incubated with 500 ng of target DNA for 20 minutes to carry out the cleavage reaction. Gel image is a representative outcome of one of three independent experiments. Values and error bars reflect mean±s.d. of n=3 independent replicates. FIG. 6F shows results of an in vitro competition-based cleavage assay examining the relative binding efficiency of a pegRNA and sgRNA for Cas9. 500 ng of the PCR product was used for the DNA target, 5 pmol of Cas9 protein was complexed with 10 pmol of sgRNA or pegRNA for 20 minutes followed by competition with 10 pmol of competing sgRNA wherever applicable (see table describing the contents of each lane). The resulting RNP complex was incubated with 500 ng of appropriate target DNA for 20 minutes to carry out the cleavage reaction. Lane 10 shows that when the mCherry sgRNA is loaded first on Cas9 and then competed with an sgRNA targeting the AAVS1 site, it is able to cleave the AAVS1 PCR product marginally. By contrast, in lane 6, when the mCherry pegRNA is loaded on Cas9 first and then competed with the AAVS1 sgRNA, it cleaves the AAVS1 PCR product to a greater extent. Gel image is a representative outcome of one of three independent experiments. FIG. 6G is a bar graph showing quantification of the cleavage products from Lanes 6, 10, and 11 of FIG. 6F indicating that AAVS1 sgRNA is able to effectively outcompete mCherry pegRNA to Cas9 when compared to the mCherry sgRNA. Values and error bars reflect mean±s.d. of n=3 independent replicates. Comparison of mean values was conducted with unpaired, two-tailed Student's t-test, **** indicates P≤0.0001.

FIG. 7A shows prime editing efficiencies at FANCF (+5 G to T) titrating different amounts of expression plasmids for PEmax and pegRNA, delivered by transient transfection to 100k HEK293T cells. The mass ratio of PEmax-encoding plasmid and pegRNA-encoding plasmid were maintained at 2:1. Editing efficiency reflects the frequency of sequencing reads that contain the intended precise edit among all sequencing reads from amplicon deep sequencing. Values and error bars reflect mean±s.d. of n=3 independent biological replicates. FIG. 7B is a bar graph showing prime editing efficiencies at FANCF (+5 G to T) using PEmax RNP programmed with pegRNA, delivered by electroporation to HEK293T cells. FIG. 7C is a bar graph showing prime editing efficiencies at HEK4 (+5 G to T) using PEmax RNP programmed with pegRNA, delivered by electroporation to HEK293T cells. The molar ratio of PE protein:pegRNA delivered was varied between 1:2 and 1:10, maintaining the PE protein at 50 pmol. Editing efficiency reflects the frequency of sequencing reads that contain the intended edit or others (indels and imprecise prime editing) among all sequencing reads from amplicon deep sequencing. Values and error bars reflect mean±s.d. of n=3 independent biological replicates. One-way ANOVA statistical analyses were used to compare the intended editing from different molar ratios of PE protein:pegRNA, 100 pmol pegRNA group was used as a control column for multiple comparisons. ns indicates P>0.05, * indicates P≤0.05, ** indicates P≤0.01, and **** indicates P≤0.0001. FIG. 7D is a plot comparing precise prime editing rates at FANCF (+5 G to T) between three different delivery platforms (transfection of expression plasmids encoding the prime editor and pegRNA, or electroporation of PE mRNA, or RNP with synthetic pegRNAs) in HEK293T cells from the experiments of FIG. 1D-F.

FIG. 8A shows prime editing strategies to correct a T158M mutation in MECP2, to disrupt the GATA1 binding motif of BCL11A erythroid enhancer, and to create a +5 G→T mutation at a HEK4 target site. Asterisked sequences denote the PAM and underlined sequences denote the spacer region of a corresponding pegRNA. Nucleotides in lowercase denote the edit incorporated. Dashes indicate deletions. FIG. 8A discloses SEQ ID NOS 8-13, respectively, in order of appearance. FIG. 8B is a bar graph showing efficiency of correction of the T158M mutation at MECP2 in HEK293T cells with a panel of pegRNAs having different PBS lengths. FIG. 8C is a bar graph showing efficiency of disruption of the GATA1 binding motif at BCL11A in HEK293T cells with a panel of pegRNAs having different PBS lengths. FIG. 8D is a bar graph showing efficiency of FANCF+5G→T editing in U20S cells with a panel of pegRNAs having different PBS lengths. 50 pmol PEmax protein and 200 pmol pegRNA were used for RNP electroporation. Editing efficiency reflects the frequency of sequencing reads that contain the intended prime editing or others (indels and imprecise prime editing) among all sequencing reads from amplicon deep sequencing. Values and error bars reflect mean±s.d. of n=3 independent replicates. One-way ANOVA statistical analyses were used to compare the intended editing from different pegRNA, where the pegRNA with 13 nt PBS group was used as a control column for multiple comparisons. ns indicates P>0.05, * indicates P≤0.05, ** indicates P≤0.01, *** indicates P≤0.001, and **** indicates P≤0.0001. FIG. 8E is a bar graph showing editing efficiency using PE3 to introduce FANCF+5G→T edits in U20S cells with different concentrations of the nicking guide. FIG. 8F is a bar graph showing editing efficiency using PE3 to introduce HEK4+5G→T edits in U2OS cells with different concentrations of the nicking guide. The amount of PEmax protein (50 μmol) and pegRNA (200 μmol) was held constant while increasing the amount of nk sgRNA delivered by electroporation. Editing efficiency reflects the frequency of sequencing reads that contain the intended prime editing or others (indels and imprecise prime editing) among all sequencing reads from amplicon deep sequencing. Values and error bars reflect mean±s.d. of n=3 independent replicates. One-way ANOVA statistical analyses were used to compare the intended edit from different amounts of nicking sgRNAs, PE2 group was used as a control column for multiple comparisons. ns indicates P>0.05, * indicates P≤0.05, ** indicates P≤0.01, *** indicates P≤0.001, and **** indicates P≤0.0001.

FIG. 9A is a graph indicating calculated Tm for the interaction between the nicked 3′ DNA end produced by the prime editor at the mCherry stop codon locus, and the PBS region of corresponding pegRNA. FIG. 9B is a graph indicating calculated Tm for the interaction between the nicked 3′ DNA end produced by the prime editor at the FANCF +5G→T locus, and the PBS region of corresponding pegRNA. FIG. 9C is a graph indicating calculated Tm for the interaction between the nicked 3′ DNA end produced by the prime editor at the BCL11A GATA1 disruption locus, and the PBS region of corresponding pegRNA. FIG. 9D is a graph indicating calculated Tm for the interaction between the nicked 3′ DNA end produced by the prime editor at the MECP2 locus for correction of the T158M mutation, and the PBS region of corresponding pegRNA. Tms were calculated using the MELTING 5 software package for RNA-DNA hybrids(35). Tins are displayed as a function of the precise editing rate at each of the loci (mCherry stop codon, FANCF+5G→T, BCL11A GATA1 disruption and correction of the T158M mutation at MECP2) in HEK293T cells. At each of the four loci, the highest editing rate is observed for a calculated Tm of approximately 37° C. FIG. 9E is a graph of MELTING 5-predicted Tins for pegRNAs having different PBS lengths targeting SBDS IVS2+2T>C for the interaction between the nicked 3′ DNA end produced by the prime editor and the PBS region of corresponding pegRNA. FIG. 9F is a graph of MELTING 5-predicted Tins for pegRNAs having different PBS lengths targeting +5G→T HEK4 for the interaction between the nicked 3′ DNA end produced by the prime editor and the PBS region of corresponding pegRNA. pegRNA designs targeting these loci were chosen based on PBS lengths that have a predicted Tm ˜37° C. (9 nt PBS length for SBDS pegRNA, and 8 nt PBS length for HEK4 pegRNA). FIG. 9G shows a strategy for correction of SBDS IVS2+2T>C. +2 indicates the position of the transition mutation in intron 2 of SBDS, not the position of base conversion relative to the prime editor cleavage site. Asterisks denote the PAM sequence and the underlined sequence denotes the spacer region of the pegRNA. Nucleotides in lowercase denote the edit incorporated. FIG. 9G discloses SEQ ID NOS 14-15, respectively, in order of appearance. FIG. 9H is a bar graph showing editing rates at the SBDSP1 target site with a pegRNA that was designed based on the Tm prediction of FIG. 9E. For PE2, 50 pmol PEmax protein and 200 pmol pegRNA were used for RNP electroporation. For PE3, 50 pmol PEmax protein, 200 pmol pegRNA and 15 pmol of nicking sgRNA were used for RNP electroporation. FIG. 9I is a bar graph showing editing rates at the HEK4 target site with a pegRNA that was designed based on the Tm prediction of FIG. 9F. 50 pmol PEmax protein and 200 pmol pegRNA were used for RNP electroporation. Editing efficiency reflects the frequency of sequencing reads that contain the intended prime editing or others (indels and imprecise prime editing) among all sequencing reads from amplicon deep sequencing. Values and error bars reflect mean±s.d. of n=3 independent biological replicates. Comparison of PE2 and PE3 mediated intended edit for SBDSP1 was conducted with unpaired, two-tailed Student's t-test, **** indicates P≤0.0001.

FIG. 10A shows a strategy for introduction of a Tek R841W mutation. The asterisked sequence denotes the PAM and the arginine codon to be changed. The lowercase nucleotides denote the edit incorporated. FIG. 10A discloses SEQ ID NOS 16-17, respectively, in order of appearance. FIG. 10B is a bar graph comparing PE2 and PE3 prime editing approaches using pegRNAs with different PBS lengths (6 and 7 nt) that introduce the R841W mutation at the tek locus in zebrafish. For PE2, 12 M pegRNA and 6 μM PE protein were combined in nuclease-free water. For PE3 a nicking sgRNA was added to the PE2 complex at a 1 to 10 nicking sgRNA to pegRNA molar ratio. Editing efficiency reflects the frequency of sequencing reads that contain the intended precise edit or others (indels and imprecise prime editing) among all sequencing reads from amplicon deep sequencing. Values and error bars reflect mean±s.d. of n=3 independent biological replicates. Comparisons of mean values of PE2- and PE3-mediated precise editing for the two different pegRNA were conducted with unpaired, two-tailed Student's t-test, * indicates P≤0.05.

FIG. 11 shows a comparison of prime editing efficiency at 30° C. and 37° C. across multiple loci, different cell types, and different delivery methods (mRNA and RNP), Comparison of mean values was conducted with paired, two-tailed Student's t-test; ** stands for P≤0.01.

FIG. 12 shows the protein sequence of PEmax used for bacterial expression and purification. FIG. 12 discloses SEQ ID NO: 18.

FIG. 13A shows hydrogen bonding for various base pairings of uracil and adenosine, and exemplary self-avoiding bases 2-thiouracil and 2-aminopurine, in accordance with embodiments of the invention. FIG. 13B shows hydrogen bonding for various base pairings of cytosine and guanine, and exemplary self-avoiding bases N4-ethyl-cytosine and hypoxanthine, in accordance with embodiments of the invention. Adapted from Hoshika et al. Nucleic Acids Symp Ser (Oxf). 2008; (52):129-30.

FIG. 14A is a drawing depicting various regions of pegRNA, in accordance with embodiments of the invention. Adapted from Synthego (www.synthego.com/guide/crispr-methods/prime-editing). FIG. 14B is a drawing showing domains of an exemplary prime editor protein, in accordance with embodiments of the invention. FIG. 14C is a drawing showing an exemplary ribonucleoprotein complex (RNP) comprising a prime editor protein bound to a pegRNA, in accordance with embodiments of the invention. FIG. 14D is a schematic of a mechanism of prime editing for PE2 prime editing systems, in accordance with embodiments of the invention. FIGS. 14B-D from Synthego (www.synthego.com/guide/crispr-methods/prime-editing).

### DETAILED DESCRIPTION OF SPECIFIC EMBODIMENTS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

“Nucleobase” or “base,” as used herein, means a group of atoms that can be linked to a sugar moiety to create a nucleoside that is capable of incorporation into a nucleic acid molecule as a nucleotide, and wherein the group of atoms is capable of hydrogen bonding with a complementary nucleobase. Nucleobases may be naturally occurring or may be modified. “Nucleobase” and “base” are used interchangeably herein.

As used herein, “unmodified nucleobase,” “unmodified base,” “naturally occurring nucleobase,” “naturally occurring base,” and the like, means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” or “modified base” means any nucleobase that is not a naturally occurring nucleobase. As used herein, 5-methyl cytosine is not a “modified nucleobase”. Although hypoxanthine is a naturally occurring purine derivative (the base of the nucleoside inosine), as used herein, hypoxanthine is a modified nucleobase and is not a naturally occurring nucleobase.

As used herein, “self-avoiding base” or “self-avoiding nucleobase” means a modified base configured to form a more stable base pair with its corresponding complementary naturally occurring base (also referred to as a “complement naturally occurring base” or “natural complement”) than with its corresponding complementary self-avoiding base (also referred to as a “complement self-avoiding base”), based on the number and strength of hydrogen bonds formed by the base pair, as shown in Table 1 (Hoshika et al. Nucleic Acids Symp Ser (Oxf). 2008; (52):129-30).

TABLE 1
Self-avoiding bases and hydrogen bonds formed by base pairing

| First Base of Pair | Corresponding Second Base of Pair | Base Pair Hydrogen Bonds |
|---|---|---|
| 2-Thiouracil (self-avoiding base) | 2-Aminopurine (complement self-avoiding base) | 1 hydrogen bond |
| 2-Aminopurine (self-avoiding base) | 2-Thiouracil (complement self-avoiding base) | 1 hydrogen bond |
| 2-Thiouracil (self-avoiding base) | Adenosine (natural complement) | 2 hydrogen bonds |
| 2-Aminopurine (self-avoiding base) | Uracil (natural complement) | 2 hydrogen bonds |
| 2-Aminopurine (self-avoiding base) | Thymine (natural complement) | 2 hydrogen bonds |
| N4-ethyl-cytosine (self-avoiding base) | Hypoxanthine (complement self-avoiding base) | 1 hydrogen bond (and |
| | | 1 weak hydrogen bond) |
| N4-methyl-cytosine (self-avoiding base) | Hypoxanthine (complement self-avoiding base) | 1 hydrogen bond (and |
| | | 1 weak hydrogen bond) |
| Hypoxanthine (self-avoiding base) | N4-ethyl-cytosine (complement self-avoiding base) | 1 hydrogen bond (and |
| | | 1 weak hydrogen bond) |
| Hypoxanthine (self-avoiding base) | N4-methyl-cytosine (complement self-avoiding base) | 1 hydrogen bond (and |
| | | 1 weak hydrogen bond) |
| N4-ethyl-cytosine (self-avoiding base) | Guanine (natural complement) | 2 hydrogen bonds (and |
| | | 1 weak hydrogen bond) |
| N4-methyl-cytosine (self-avoiding base) | Guanine (natural complement) | 2 hydrogen bonds (and |
| | | 1 weak hydrogen bond) |
| Hypoxanthine (self-avoiding base) | Cytosine (natural complement) | 2 hydrogen bonds |

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease.

CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S. A. 98:4658-4663(2001); “CRISPR RNA maturation by trans-encoded small RNA and host factor Rnase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilis. Additional suitable Cas9 nucleases and sequences will be apparent to those of skill in the art based on U.S. Pat. No. 11,447,770, which is hereby incorporated by reference for its disclosure of Cas9 nucleases, and such Cas9 nucleases and Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737, the entire contents of which are incorporated herein by reference. In some embodiments, a Cas9 nuclease comprises one or more mutations that partially impair or inactivate the DNA cleavage domain. U.S. Pat. No. 11,447,770 is hereby incorporated by reference for its disclosure of Cas9.

The term “nickase,” “Cas9 nickase,” and “nCas9” refers to a Cas9 with one of its two nuclease domains inactivated. This enzyme is capable of cleaving only one strand of a target DNA. U.S. Pat. No. 11,447,770 is hereby incorporated by reference for its disclosure of Cas9 nickases.

As used herein, the terms “DNA synthesis template,” “reverse transcriptase template,” and “RTT” are used interchangeably to refer to the region or portion of the extension arm of a pegRNA that is utilized as a template strand by a polymerase, such as reverse transcriptase, of a prime editor to encode a 3′ replacement DNA flap that contains a desired edit and which then, through the mechanism of prime editing, replaces the corresponding endogenous strand of DNA at the target site. U.S. Pat. No. 11,447,770 is hereby incorporated by reference for its disclosure of DNA synthesis templates, the mechanisms of prime editing, components of prime editing systems, and methods of using prime editors and prime editing systems.

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

The term “extension arm” refers to a nucleotide sequence component of a pegRNA which provides several functions, including a primer binding site (PBS) and a DNA synthesis template (also referred to as an “reverse transcriptase template” or “RTT”) for reverse transcriptase. In some embodiments the extension arm is located at the 3′ end of the guide RNA. In various embodiments, the extension arm comprises the following components in a 5′ to 3′ direction: the DNA synthesis template and the primer binding site. Since polymerization activity of the reverse transcriptase is in the 5′ to 3′ direction, the preferred arrangement of the DNA synthesis template and primer binding site is in the 5′ to 3′ direction such that the reverse transcriptase, once primed by an annealed primer sequence, polymerases a single strand of DNA using the DNA synthesis template as a complementary template strand.

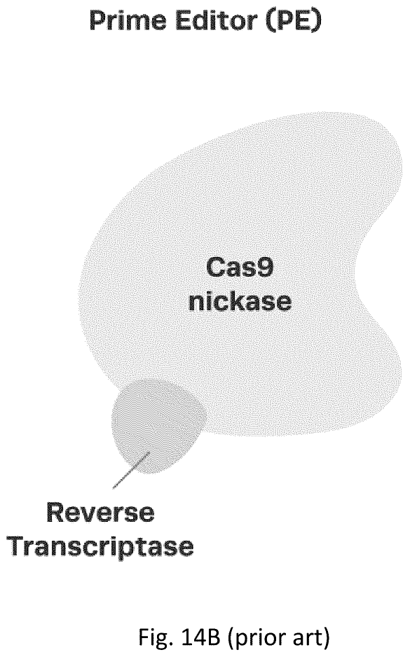

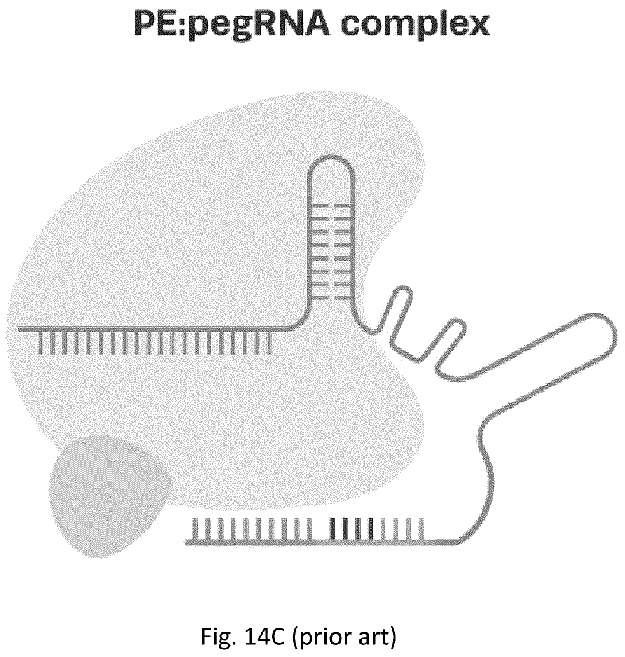

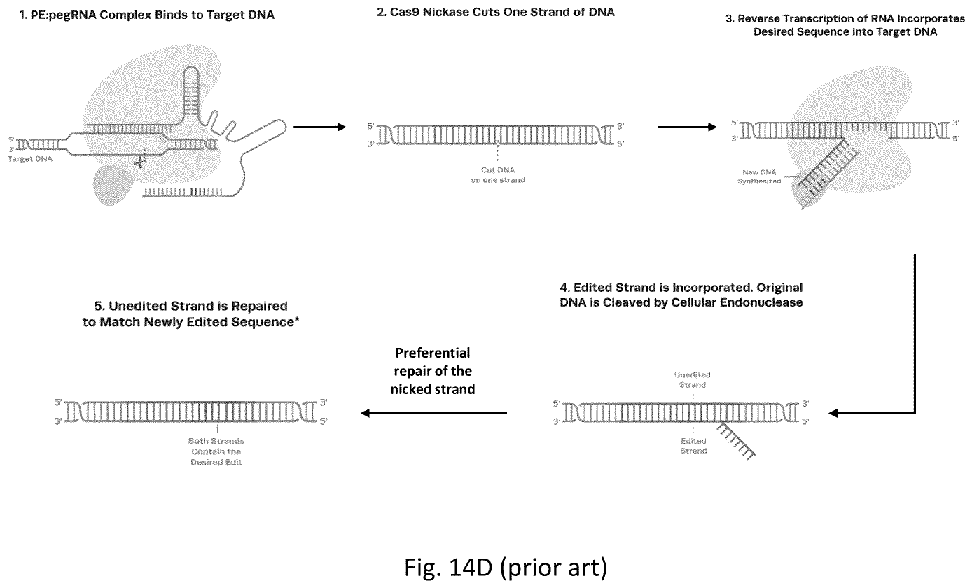

The extension arm may also be described as comprising generally two regions: a primer binding site (PBS) and a DNA synthesis template, as shown in FIG. 14A. The primer binding site binds to a primer sequence that is formed from the endogenous DNA strand of the target site when it becomes nicked by the prime editor complex, thereby exposing a 3′ end on the endogenous nicked strand. The binding of the primer sequence to the primer binding site on the extension arm of the pegRNA creates a duplex region with an exposed 3′ end (i.e., the 3′ of the primer sequence), which then provides a substrate for reverse transcriptase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor complex and which ultimately replaces the corresponding endogenous DNA strand of the target site that sits immediate downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the pegRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA. U.S. Pat. No. 11,447,770 is hereby incorporated by reference for its disclosure of extension arms.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. For example, a fusion protein may comprise a Cas9 nickase fused to a reverse transcriptase. Such a fusion protein is referred to herein as a “prime editor protein.” Certain embodiments of a prime editor protein include the embodiment of FIG. 14B.

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity a spacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. “Prime editing guide RNA” (or “pegRNA”) is a guide RNA that has been modified and designed for the prime editing methods and systems disclosed herein. A pegRNA associates with a prime editor protein. U.S. Pat. No. 11,447,770 is hereby incorporated by reference for its disclosure of guide RNA.

A pegRNA may comprise various structural elements that include, but are not limited to:

Spacer sequence—the sequence in the pegRNA (in some embodiments, having about 20 nts in length) which binds to the protospacer (target sequence) in the target DNA.

gRNA core (or gRNA scaffold or backbone sequence)—refers to the sequence within the gRNA that is responsible for Cas9 binding, it does not include a spacer sequence that is used to guide Cas9 to target DNA.

Extension arm—a single strand extension at the 3′ end of the pegRNA which comprises a primer binding sequence and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change.

Transcription terminator—the pegRNA may comprise a transcriptional termination sequence at the 3′ of the molecule.

The term “homology arm” refers to a portion of the extension arm that encodes a portion of the resulting reverse transcriptase-encoded single strand DNA flap that is to be integrated into the target DNA site by replacing the endogenous strand. The portion of the single strand DNA flap encoded by the homology arm is complementary to the non-edited strand of the target DNA sequence, which facilitates the displacement of the endogenous strand and annealing of the single strand DNA flap in its place, thereby installing the edit. The homology arm is part of the DNA synthesis template since it is by definition introduced into the target DNA by the polymerase of the prime editors described herein. U.S. Pat. No. 11,447,770 is hereby incorporated by reference for its disclosure of homology arms.

As used herein, the term “nucleic acid programmable DNA binding protein” or “napDNAbp,” of which Cas9 is an example, refer to a proteins which use RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., a spacer sequence of a guide RNA). In other words, the guide nucleic-acid “programs” the napDNAbp (e.g., Cas9 or equivalent) to localize and bind to a complementary sequence. U.S. Pat. No. 11,447,770 is hereby incorporated by reference for its disclosure of napDNAbps.

The term “nuclear localization sequence,” “nuclear localization signal,” “nuclear localization signal sequence,” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, a NLS comprises the amino acid sequence PKKKRKV (SEQ ID NO: 1) or MDSLLMNRRKFLYQFKNVRWAKGRRETYLC (SEQ ID NO: 2).

As used herein, the terms “prime editing guide RNA” or “pegRNA” refers to a specialized form of a guide RNA that has been modified to include one or more additional sequences for implementing the prime editing methods and systems described herein. The additional sequences comprise (i) a “DNA synthesis template” which encodes (copied by the polymerase, e.g., reverse transcriptase, of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA; and (ii) a “primer binding site.” As used herein the “primer binding site” comprises a sequence that hybridizes to a single-strand DNA sequence having a 3′ end generated from the nicked DNA of the R-loop. U.S. Pat. No. 11,447,770 is hereby incorporated by reference for its disclosure of pegRNA.

In certain embodiments, a structure of a pegRNA is represented by FIG. 14A, which shows a pegRNA having a 5′ extension arm, a spacer sequence, and a gRNA core. The 5′ extension further comprises in the 5′ to 3′ direction a DNA synthesis template (reverse transcriptase template) and a primer binding site.

As used herein, “PE2” refers to a PE complex comprising a fusion protein comprising a Cas9 nickase and a reverse transcriptase (RT), and a desired pegRNA, e.g., a fusion protein comprising Cas9(H840A) and a variant MMLV RT having the structure: [NLS]-[Cas9(H840A)]-[linker]-[MMLV_RT(D200N)(T330P)(L603W)(T306K)(W313F)]+a desired pegRNA. Certain embodiments of a PE complex include the embodiment of FIG. 14C. U.S. Pat. No. 11,447,770 is hereby incorporated by reference for its disclosure of PE2.

As used herein, “PE3” refers to PE2 plus a second-strand nicking guide RNA (Nk sgRNA) that complexes with the PE2 and introduces a nick in the non-edited DNA strand in order to induce preferential replacement of the edited strand. U.S. Pat. No. 11,447,770 is hereby incorporated by reference for its disclosure of nicking guide RNA, second-strand nicking, and PE3.

As used herein, the term “polymerase” refers to an enzyme that synthesizes a nucleotide strand and which may be used in connection with the prime editor systems described herein. Reverse transcriptase is a polymerase. U.S. Pat. No. 11,447,770 is hereby incorporated by reference for its disclosure of polymerases.

As used herein, the term “prime editing” refers to an approach for gene editing using napDNAbps (e.g., a Cas9 nickase), a polymerase (e.g., a reverse transcriptase), and specialized guide RNAs that include a DNA synthesis template for encoding desired new genetic information (or deleting genetic information) that is then incorporated into a target DNA sequence. Certain embodiments of prime editing are shown in the embodiment of FIG. 14D. U.S. Pat. No. 11,447,770 is hereby incorporated by reference for its disclosure of prime editing.

The term “prime editor protein” refers to fusion constructs comprising a napDNAbp (e.g., Cas9 nickase) and a polymerase (e.g., reverse transcriptase) and is capable of carrying out prime editing on a target nucleotide sequence in the presence of a pegRNA.

The term “prime editor” may refer to the fusion protein or to the fusion protein complexed with a pegRNA, and/or further complexed with a second-strand nicking sgRNA. U.S. Pat. No. 11,447,770 is hereby incorporated by reference for its disclosure of prime editors and prime editor proteins.

The terms “primer binding site,” “primer binding sequence,” and “PBS” are used interchangeably to refer to the nucleotide sequence located on a pegRNA as component of the extension arm (typically at the 3′ end of the extension arm) and serves to bind to the primer sequence that is formed after Cas9 nicking of the target site sequence by the prime editor. U.S. Pat. No. 11,447,770 is hereby incorporated by reference for its disclosure of primer binding sites.

The term “reverse transcriptase” describes a class of polymerases characterized as RNA-dependent DNA polymerases. All known reverse transcriptases require a primer to synthesize a DNA transcript from an RNA template. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. U.S. Pat. No. 11,447,770 is hereby incorporated by reference for its disclosure of reverse transcriptases.

The term “target site” refers to a sequence within a nucleic acid molecule that is edited by a prime editor (PE) disclosed herein. The target site further refers to the target sequence within a nucleic acid molecule to which a complex of the prime editor (PE) and gRNA binds.

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

As used herein, the term “3′ replacement DNA flap” or simply, “replacement DNA flap,” refers to the strand of DNA that is synthesized by the prime editor and which is encoded by the extension arm of the prime editor pegRNA. More in particular, the 3′ replacement DNA flap is encoded by the DNA synthesis template of the pegRNA. The 3′ replacement DNA flap comprises the same sequence as the 5′ endogenous DNA flap except that it also contains the edited sequence (e.g., single nucleotide change). The 3′ replacement DNA flap anneals to the target DNA, displacing or replacing a 5′ endogenous DNA flap (which can be excised, for example, by a 5′ flap endonuclease, such as FEN1 or EXO1) and then is ligated to join the 3′ end of the 3′ replacement DNA flap to the exposed 5′ hydoxyl end of endogenous DNA (exposed after excision of the 5′ endogenous DNA flap, thereby reforming a phosophodiester bond and installing the 3′ replacement DNA flap to form a heteroduplex DNA containing one edited strand and one unedited strand. DNA repair processes resolve the heteroduplex by copying the information in the edited strand to the complementary strand permanently installs the edit into the DNA. This resolution process can be driven further to completion by nicking the unedited strand, i.e., by way of “second-strand nicking,” U.S. Pat. No. 11,447,770 is hereby incorporated by reference for its disclosure of 3′ replacement DNA flaps, 5′ endogenous DNA flaps, and 5′ endogenous DNA flap removal, and second-strand nicking.

“Engineered pegRNA” or “epegRNA” is a pegRNA comprising a 3′ structured RNA pseudoknot (3′ of the PBS of the pegRNA) that protects the 3′ extension arm from degradation by exonucleases. epegRNAs are described in Liu et al., International PCT Application PCT/US2021/052097, filed Sep. 24, 2021, published as WO/2022067130 on Mar. 31, 2022, the contents of which are incorporated herein by reference for its disclosure of epegRNA.

The prime editor proteins disclosed herein form a complex with (e.g., bind or associate with) one or more RNA(s) that is not a target for cleavage. In some embodiments, an RNA-programmable nuclease, such as a prime editor protein, when in a complex with an RNA, may be referred to as a ribonucleoprotein complex, ribonucleoprotein, RNP, or RNP complex. The bound RNA(s) may be, for example, a pegRNA, an epegRNA, or a gRNA. Prime editor protein-pegRNA complexes may be referred to as PE RNPs.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

“Melting temperature” (“Tm”) is the temperature at which one half of the strands of a population of duplexed nucleic acid will dissociate to become single-stranded. In some embodiments, the duplexed nucleic acid is a RNA:DNA duplex. Melting temperature is a function of both the sequence and the length of the duplex. Methods of calculating Tm are well known in the art. See, e.g., Dumousseau et al. (2012) BMC Bioinformatics, 13, 101, hereby incorporated by reference for its disclosure of melting temperature calculation.

By “hybridizable” (and derivatives thereof) or “complementary” it is meant that a nucleic acid (e.g. RNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes; adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary. U.S. Publication No. US 2019/0010520 is hereby incorporated by reference for its disclosure of complementarity (including “complementary”) and hybridization (including “hybridizable”).

“Auto-inhibition,” as used herein, refers to the restriction of prime editing efficiency of prime-editing systems caused by intramolecular interactions of a pegRNA of the prime-editing system, the intramolecular interactions being a consequence of the inherent complementarity, and thus base pairing potential, of the PBS (or the PBS and at least a portion of the RTT) with the spacer sequence of the pegRNA.

Prime editors, e.g., PE2 systems, rewrite genomic sequence in a targeted manner through a multi-step process: [1] recognition of the target sequence through the spacer sequence encoded at the 5′ end of the pegRNA; [2] Nicking of the non-target DNA strand upon R-loop formation by the Cas9 nickase; [3] Annealing of the free 3′ end of the nicked DNA to the primer binding site (PBS) at the 3′ of the pegRNA; [4] Extension of the free 3′ DNA end by MMLV-RT appending the sequence defined by the reverse transcriptase template (RTT) region of the pegRNA; and [5] Incorporation of the extended DNA sequence into the genome through endogenous DNA repair pathways, which can be facilitated by sequence homology (Homology arm (HA)) encoded within the RTT. Rates of precise repair (successful template-dependent prime editing) can be increased by multiple approaches, including: prime editors with improved efficiency (3-6), pegRNA designs with improved stability(7-9), the introduction of a nick (PE3)(1) or second prime editor complex editing the opposite DNA strand(9-13), and inhibition of DNA repair factors (PE4 & PE5) that disfavor the incorporation of prime editor DNA products into the genome(6,14).

Utilization of prime editing systems to enable genome alteration has been primarily focused on DNA(1, 4, 15), RNA(6, 8, 16, 17) or viral delivery(4, 18-20). Prime editor protein-pegRNA complexes (PE RNPs) have been successfully employed in transformed cell lines, zebrafish embryos, primary human T cells and induced pluripotent cells(21,22). However, in general, precise editing rates in these studies were modest (>10%) compared to the editing rates that have been achieved by other delivery methodologies.

Here, we disclose that the inherent complementarity between the PBS and spacer sequence within a pegRNA restricts the efficiency of prime editing. This complementarity can extend into the first 3 nucleotides (nt) of the RTT region if it is identical to the DNA target site. Because RNA-RNA duplexes are typically more stable than RNA-DNA duplexes(23), and due to the intramolecular nature of the association between the spacer and PBS regions of the pegRNA, the formation of a PBS-spacer RNA duplex can preclude the formation of an R-loop by the prime editor at its target site. Finding the correct balance between PBS length and pegRNA sequence composition is critical to maximize prime editing activity by reducing this inherent “auto-inhibition” within a pegRNA sequence. Reducing complementarity/hybridization between a PBS and a spacer sequence within a pegRNA reduces this inherent auto-inhibition, caused by interaction of the PBS (or PBS and a portion of the RTT) with the spacer sequence, and improves prime editing activity of PE RNPs across multiple target sites and multiple cell types.

In some embodiments, a pegRNA comprises a modification conferring resistance to nuclease degradation. The pegRNA may comprise at least one nucleotide comprising the modification conferring resistance to nuclease degradation. In some embodiments, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

In some embodiments, reduced complementarity/hybridization between a PBS and a spacer sequence may be achieved by shortening PBS length in an end-protected pegRNA, thereby reducing auto-inhibition. We disclose here that end-protected pegRNAs require shorter PBS lengths than non-end-protected pegRNAs for efficient prime editing. An end protected pegRNA comprises one or more modifications conferring resistance to nuclease degradation. The modifications may be at a 3′ series of 1-50 nucleotides of a 3′ terminus of a pegRNA, at a 5′ series of 1-50 nucleotides of a 5′ terminus of the pegRNA, or at both a 3′ series of 1-50 nucleotides of the 3′ terminus of the pegRNA and a 5′ series of 1-50 nucleotides of the 5′ terminus of the pegRNA. In some embodiments, the 3′ series is 1-40 nucleotides of the 3′ terminus of the pegRNA. In other embodiments, the 3′ series is 1-30 nucleotides of the 3′ terminus of the pegRNA. In some embodiments, the 3′ series is 1-20 nucleotides of the 3′ terminus of the pegRNA. In some embodiments, the 3′ series is 1-10 nucleotides of the 3′ terminus of the pegRNA. In some embodiments, the 3′ series is 3-10 nucleotides of the 3′ terminus of the pegRNA. In some embodiments, the 3′ series is 1-5 nucleotides of the 3′ terminus of the pegRNA. In some embodiments, the 5′ series is 1-50 nucleotides of the 5′ terminus of the pegRNA. In some embodiments, the 5′ series is 1-40 nucleotides of the 5′ terminus of the pegRNA. In other embodiments, the 5′ series is 1-30 nucleotides of the 5′ terminus of the pegRNA. In some embodiments, the 5′ series is 1-20 nucleotides of the 5′ terminus of the pegRNA. In some embodiments, the 5′ series is 1-10 nucleotides of the 5′ terminus of the pegRNA. In some embodiments, the 5′ series is 3-10 nucleotides of the 5′ terminus of the pegRNA. In some embodiments, the 5′ series is 1-5 nucleotides of the 5′ terminus of the pegRNA. In some embodiments, the 3′ series of nucleotides is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, and 50 nucleotides of the 3′ terminus of the pegRNA. In some embodiments, the 5′ series of nucleotides is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, and 50 nucleotides of the 5′ terminus of the pegRNA.

In some embodiments, independently for any given nucleotide of the 5′ series and/or the 3′ series, the modification conferring resistance to nuclease degradation comprises a phosphorothioate modification. In other embodiments, independently for any given nucleotide of the 5′ series and/or the 3′ series, the modification conferring resistance to nuclease degradation comprises a 2′ modification selected from the group consisting of a 2′ O-methyl modification, a 2′-fluoro modification, a 2′-O-methoxyethyl (2′-MOE) modification, a 2′-deoxy modification, and a 2′-amino modification. In some embodiments, independently for any given nucleotide of the 5′ series and/or the 3′ series, the modification conferring resistance to nuclease degradation comprises both (i) a phosphorothioate modification and (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification. Additional modifications known to those of skill in the art to confer resistance to nuclease degradation are also contemplated as part of the present disclosure.

In some embodiments, the PBS of a pegRNA in accordance with embodiments of the invention is 5-20 nucleotides in length. In other embodiments, the PBS of the pegRNA is 5-15 nucleotides in length. In some embodiments, the PBS of the pegRNA is 5-10 nucleotides in length. In other embodiments, the PBS of the pegRNA is 6-8 nucleotides in length. In some embodiments, the PBS of the pegRNA consists of a number of nucleotides selected from the group consisting of 5, 6, 7, 8, 9, 10, 11, 12, 13, 14, 15, 16, 17, 18, 19, and 20 nucleotides.

In some embodiments, reduced complementarity/hybridization between a PBS and a spacer sequence may be achieved using a pegRNA comprising an extension, 3′ of the PBS of the pegRNA, that is complementary to the PBS, or complementary to the PBS and to at least a portion of the RTT, of the pegRNA, thereby forming a hairpin with the primer binding sequence (or with the PBS and at least a portion of the RTT). The 3′ extension, by preferentially hybridizing with the PBS, or with the PBS and with at least a portion of the RTT, thereby forming a hairpin structure, reduces potential hybridization of the PBS with the spacer sequence, thereby reducing auto-inhibition. In some embodiments, the 3′ extension is 5-25 nucleotides in length. In other embodiments, the 3′ extension is 5-20 nucleotides in length. In some embodiments, the 3′ extension is 5-15 nucleotides in length. In other embodiments, the 3′ extension is 5-10 nucleotides in length. In some embodiments, the 3′ extension consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

In other embodiments, a DNA-competing oligonucleotide (also referred to as a competing oligonucleotide) complementary to the PBS, or complementary to the PBS and to at least a portion of the RTT, of a pegRNA, co-administered to a cell along with an RNP comprising a prime editor protein in complex with the pegRNA, may be used to reduce hybridization between the PBS (and potentially a portion of the RTT) and the spacer sequence of the pegRNA. The competing oligonucleotide, by preferentially hybridizing with the PBS, or with the PBS and with at least a portion of the RTT, reduces potential hybridization of the PBS with the spacer sequence, thereby reducing auto-inhibition. In some embodiments, the competing oligonucleotide complementary to the PBS, or complementary to the PBS and to at least a portion of the RTT, is 5-25 nucleotides in length. In other embodiments, the competing oligonucleotide is 5-20 nucleotides in length. In some embodiments, the competing oligonucleotide is 5-15 nucleotides in length. In other embodiments, the competing oligonucleotide is 5-10 nucleotides in length. In some embodiments, the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides. In some embodiments, the competing oligonucleotide has a length, in nucleotides, selected from the group consisting of 5, 6, 7, 8, 9, 10, 11, 12, 13, 14, 15, 16, 17, 18, 19, 20, 21, 22, 23, 24, and 25.

In some embodiments, the competing oligonucleotide is an RNA oligonucleotide. In other embodiments, the competing oligonucleotide is a DNA oligonucleotide. In still other embodiments, the competing oligonucleotide comprises a ribonucleotide and a deoxyribonucleotide.

In some embodiments, at least one nucleotide of the competing oligonucleotide comprises a modification conferring resistance to nuclease degradation. In some embodiments, independently for any given nucleotide of the competing oligonucleotide, the at least one modification conferring resistance to nuclease degradation comprises a phosphorothioate modification. In other embodiments, independently for any given nucleotide of the competing oligonucleotide, the at least modification conferring resistance to nuclease degradation comprises a 2′ modification selected from the group consisting of a 2′ O-methyl modification, a 2′-fluoro modification, a 2′-O-methoxyethyl (2′-MOE) modification, a 2′-deoxy modification, and a 2′-amino modification. In still other embodiments, independently for any given nucleotide of the competing oligonucleotide, the at least modification conferring resistance to nuclease degradation comprises both (i) a phosphorothioate modification and (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification. Additional modifications known to those of skill in the art to confer resistance to nuclease degradation are also contemplated as part of the present disclosure.

In some embodiments, reduced complementarity between PBS and spacer sequences may be achieved by incorporating at least one self-avoiding base into the PBS, and at least one corresponding complement self-avoiding base into the spacer sequence, of a pegRNA, thereby reducing auto-inhibition. In some embodiments, a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

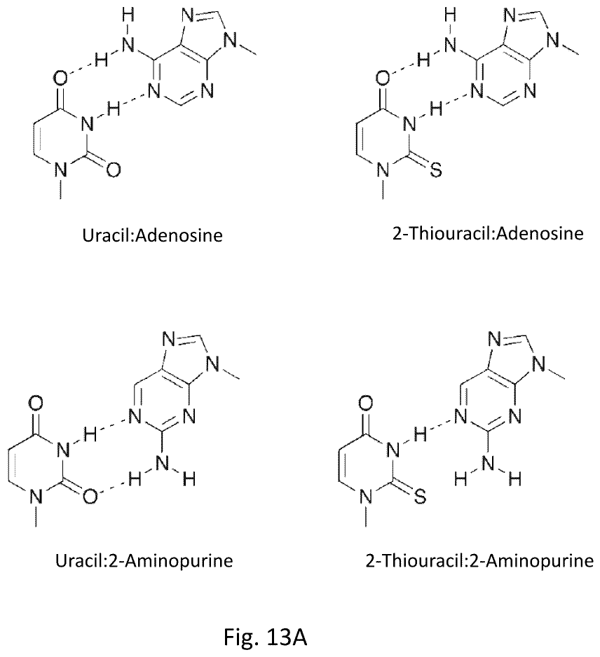

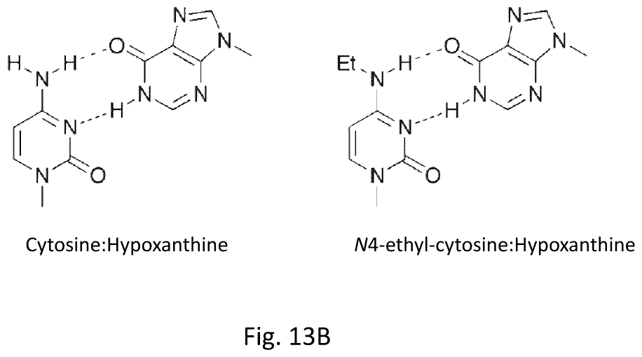

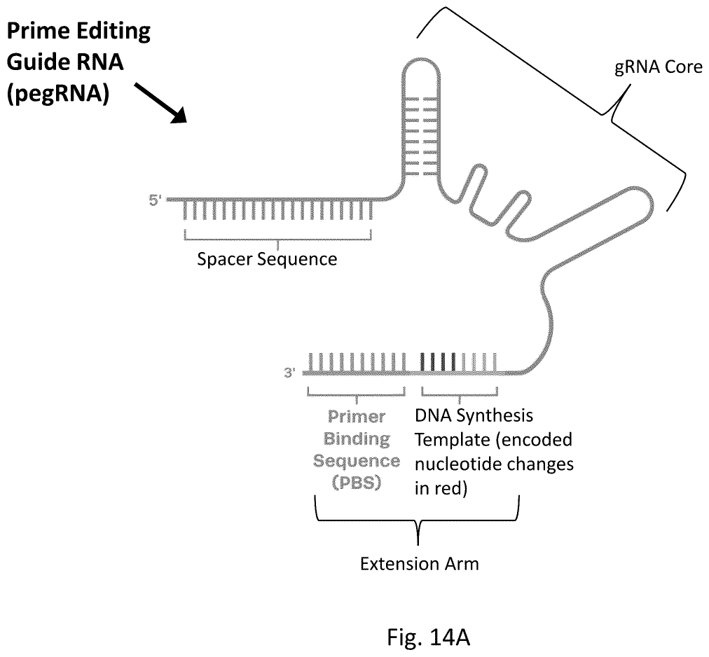

A self-avoiding base forms a more stable base pair with a complementary naturally occurring base than with its corresponding complement self-avoiding base (FIG. 13A and FIG. 13B). Thus, for example, when a nucleotide of the PBS of the pegRNA comprises a self-avoiding base, and a nucleotide of the spacer sequence of the pegRNA comprises a corresponding complement self-avoiding base, (i) base pairing of the self-avoiding base with the corresponding complement self-avoiding base is disfavored, i.e., hydrogen bonding is weaker, compared to base pairing of the self-avoiding base with a corresponding complementary naturally occurring base comprised by a genomic primer sequence, the primer sequence being complementary to the PBS, and (ii) base pairing of the corresponding complement self-avoiding base with the self-avoiding base is disfavored compared to base pairing of the corresponding complement self-avoiding base with a corresponding complementary naturally occurring base comprised a genomic target sequence, the target sequence being complementary to the spacer sequence. Through the use of one or more self-avoiding bases in the PBS, and one or more corresponding complement self-avoiding bases in the spacer sequence, the hybridization potential of the PBS with the spacer sequence is reduced, thereby reducing auto-inhibition. Exemplary self-avoiding bases and their corresponding complement self-avoiding bases, and corresponding complementary naturally occurring bases, are shown in Table 1.

In some embodiments, 1-20 nucleotides of the PBS of a pegRNA comprise a self-avoiding base. In other embodiments, 1-10 nucleotides of the PBS of a pegRNA comprise a self-avoiding base. In some embodiments, 1-5 nucleotides of the PBS of a pegRNA comprise a self-avoiding base. In yet other embodiments, 3-5 nucleotides of the PBS of a pegRNA comprise a self-avoiding base. In some embodiments, every nucleotide of the PBS of a pegRNA comprises a self-avoiding base.

In some embodiments, 1-30 nucleotides of the spacer sequence of a pegRNA comprise a corresponding complement self-avoiding base. In other embodiments, 1-25 nucleotides of the spacer sequence of a pegRNA comprise a corresponding complement self-avoiding base. In other embodiments, 1-20 nucleotides of the spacer sequence of a pegRNA comprise a corresponding complement self-avoiding base. In other embodiments, 1-15 nucleotides of the spacer sequence of a pegRNA comprise a corresponding complement self-avoiding base. In still other embodiments, 1-10 nucleotides of the spacer sequence of a pegRNA comprise a corresponding complement self-avoiding base. In some embodiments, 1-5 nucleotides of the spacer sequence of a pegRNA comprise a corresponding complement self-avoiding base. In yet other embodiments, 3-5 nucleotides of the spacer sequence of a pegRNA comprise a corresponding complement self-avoiding base. In some embodiments, every nucleotide of the spacer sequence of a pegRNA comprises a corresponding complement self-avoiding base.

In some embodiments, a pegRNA may further comprise an RTT comprising at least one self-avoiding base, and the spacer sequence of the pegRNA further comprises at least one corresponding complement self-avoiding base (corresponding to the at least one self-avoiding base of the RTT (i.e., a corresponding “complement RTT self-avoiding base”)), thus reducing the hybridization potential of the RTT with the spacer sequence and reducing auto-inhibition. As used herein, an “RTT self-avoiding base” means a self-avoiding base comprised by the DNA synthesis template (RTT) of a pegRNA, in accordance with embodiments of the invention. Preferably an RTT self-avoiding base is comprised by a series of RTT nucleotides adjacent to the PBS of a pegRNA.

In some embodiments, 1-20 nucleotides of the RTT, adjacent to the PBS, of a pegRNA comprise a self-avoiding base. In other embodiments, 1-10 nucleotides of the RTT, adjacent to the PBS, of a pegRNA comprise a self-avoiding base. In some embodiments, 1-5 nucleotides of the RTT, adjacent to the PBS, of a pegRNA comprise a self-avoiding base. In some embodiments, 1-3 nucleotides of the RTT, adjacent to the PBS, of a pegRNA comprise a self-avoiding base. In yet other embodiments, 3-5 nucleotides of the RTT, adjacent to the PBS, of a pegRNA comprise a self-avoiding base. In some embodiments, every nucleotide of the RTT of a pegRNA comprises a self-avoiding base.

As a non-limiting example of the use self-avoiding bases, it will be appreciated by those of skill in the art that, in some embodiments, (i) at least one adenosine of the PBS and/or RTT sequence of a pegRNA may be replaced with the self-avoiding base 2-aminopurine, with at least one uracil of the spacer sequence of the pegRNA being replaced by 2-aminopurine's corresponding complement self-avoiding base, 2-thiouracil; and/or (ii) at least one uracil of the PBS and/or RTT sequence of a pegRNA may be replaced with the self-avoiding base 2-thiouracil, with at least one adenosine of the spacer sequence of the pegRNA being replaced by 2-thiouracil's corresponding complement self-avoiding base, 2-aminopurine, and/or (iii) at least one guanine of the PBS and/or RTT sequence of a pegRNA may be replaced with the self-avoiding base hypoxanthine, with at least one cytosine of the spacer sequence of the pegRNA being replaced by hypoxanthine's corresponding complement self-avoiding base, N4-ethyl-cytosine; and/or (iv) at least one guanine of the PBS and/or RTT sequence of a pegRNA may be replaced with the self-avoiding base hypoxanthine, with at least one cytosine of the spacer sequence of the pegRNA being replaced by hypoxanthine's corresponding complement self-avoiding base, N4-methyl-cytosine; and/or (v) at least one cytosine of the PBS and/or RTT sequence of a pegRNA may be replaced with the self-avoiding base N4-ethyl-cytosine, with at least one guanine of the spacer sequence of the pegRNA being replaced by N4-ethyl-cytosine's corresponding complement self-avoiding base, hypoxanthine; and/or (vi) at least one cytosine of the PBS and/or RTT sequence of a pegRNA may be replaced with the self-avoiding base N4-methyl-cytosine, with at least one guanine of the spacer sequence of the pegRNA being replaced by N4-methyl-cytosine's corresponding complement self-avoiding base, hypoxanthine. The use of other self-avoiding bases and corresponding complement self-avoiding bases to reduce auto-inhibition are also contemplated as part of the present disclosure.

It will further be appreciated by those of skill in the art that complementarity/hybridization between the PBS and the spacer sequence of a pegRNA may be reduced through the use of one, or more than one, strategy disclosed herein for reducing auto-inhibition. For example, a pegRNA may be end-protected and also comprise self-avoiding bases. Any combination of strategies for reducing complementarity/hybridization between the PBS and the spacer sequence of the pegRNA is included as part of the present disclosure.

Moreover, we disclose that an optimal length, in nucleotides, of the PBS of a pegRNA is a length having a melting temperature (Tm) of approximately 37° C. Prime editing systems using a pegRNA comprising a PBS having a Tm of approximately 37° C. display maximum editing rates in cell culture. Remarkably, 37° C. is the temperature at which mammalian cells are typically incubated for growth during genome editing, and is close to the physiological temperature of humans.

Prime editing systems comprising a pegRNA having reduced auto-inhibition, RNPs comprising a pegRNA having reduced auto-inhibition, and pegRNA having reduced auto-inhibition, the pegRNA comprising a PBS having a Tm of approximately 37° C., are also contemplated as part of the present disclosure. Methods for calculating Tm are well known in the art. Preferably, Tm is calculated using thermodynamic analysis based on nearest-neighbor sequence composition, e.g., as described in Dumousseau et al. (2012) “MELTING, a flexible platform to predict the melting temperatures of nucleic acids”. BMC Bioinformatics, 13, 101, hereby incorporated by reference in its entirety.

In some embodiments, a pegRNA having reduced auto-inhibition, in accordance with embodiments of the invention, comprises a PBS having a Tm of 32°-42° C.

In other embodiments, a pegRNA having reduced auto-inhibition, in accordance with embodiments of the invention, comprises a PBS having a Tm of 34°-40° C. In still other embodiments, a pegRNA having reduced auto-inhibition, in accordance with embodiments of the invention, comprises a PBS having a Tm of 35°-39° C. In some embodiments, a pegRNA having reduced auto-inhibition, in accordance with embodiments of the invention, comprises a PBS having a Tm of 36°-38° C. In other embodiments, a pegRNA having reduced auto-inhibition, in accordance with embodiments of the invention, comprises a PBS having a Tm of 35.5°-38.5° C. In some embodiments, Tm is calculated using thermodynamic analysis based on nearest-neighbor sequence composition. In other embodiments, Tm is calculated using MELTING, as disclosed by Dumousseau et al. (2012) “MELTING, a flexible platform to predict the melting temperatures of nucleic acids”. BMC Bioinformatics, 13, 101. Those of skill in the art will appreciate that, for a given PBS sequence, a Tm of the PBS can be increased by increasing the length of the PBS, and a Tm of the PBS can be decreased by decreasing the length of the PBS.

In accordance with the present invention, a method for site-specific modification of a double-stranded target DNA, the method comprising contacting the double-stranded target DNA sequence with a prime editing system comprising a pegRNA having reduced auto-inhibition, is included as part of the present disclosure.

In addition, we disclose that cold shock treatment of a cell post PE RNP nucleofection significantly increases prime editing rates. A method of treating a subject having or suspected of having a disease or disorder, the method comprising administering a prime editing system or a PE RNP complex ex vivo to a cell from the subject, followed by an incubation of the cell at 10°-34° C. for a period of time, is therefore also included as part of the present disclosure. In some embodiments, the prime editing system or the PE RNP complex is administered to the cell via electroporation. In other embodiments, the prime editing system or the PE RNP complex is administered to the cell via nucleofection. In some embodiments, the prime editing system or the PE RNP complex comprises a pegRNA having reduced auto-inhibition as disclosed herein. In some embodiments, the pegRNA has a PBS having a Tm selected from the group consisting of 32°-42° C., 34°-40° C., 35°-39° C., 35.5°-38.5° C., and 36°-48° C. In some embodiments, a competing oligonucleotide complementary to a PBS, or complementary to the PBS and to at least a portion of a RTT sequence of the pegRNA, is co-administered to the cell along with the prime editing system or the PE RNP complex.

In some embodiments, the incubation of the cell is at 20°-34° C. for the period of time. In other embodiments, the incubation of the cell is at 25°-34° C. for the period of time. In some embodiments, the incubation of the cell is at 28°-32° C. for the period of time.

In other embodiments, the incubation of the cell is at 30° C. for the period of time. In some embodiments, the incubation of the cell is at a temperature, selected from the group consisting 20°±0.5° C., 21°±0.5° C., 22°±0.5° C., 23°:0.5° C., 24°±0.5° C., 25°±0.5° C., 26°±0.5° C., 27°±0.5° C., 28°±0.5° C., 29°±0.5° C., 30°±0.5° C., 31°±0.5° C., 32°±0.5° C., 33°±0.5° C., and 34°±0.5° C. for the period of time.

In some embodiments, the period of time is 1-96 hours. In other embodiments, the period of time is 1-72 hours. In some embodiments, the period of time is 12-96 hours. In other embodiments, the period of time is 12-72 hours. In some embodiments, the period of time is 24-96 hours. In other embodiments, the period of time is 24-72 hours. In some embodiments, the period of time is 48-96 hours. In other embodiments, the period of time is 48-72 hours. In some embodiments, the period of time is 12-24 hours. In other embodiments, the period of time is 12-36 hours. In some embodiments, the period of time is 24-48 hours. In other embodiments, the period of time is 24-36 hours. In some embodiments, the period of time is 36-50 hours. In other embodiments, the period of time is 36-48 hours.

The following examples are put forth so as to provide those of ordinary skill in the art with a disclosure and description of how to make and use embodiments of the present invention, and are not intended to limit the scope of the invention, nor are they intended to represent that the experiments below are all or the only experiments performed.

### Example 1: PBS and Spacer Region Interaction within pegRNA Limits Prime Editing Activity

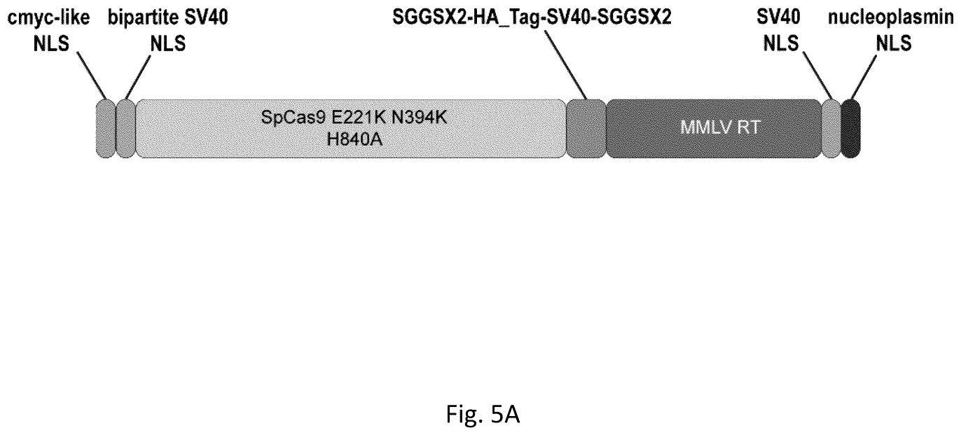

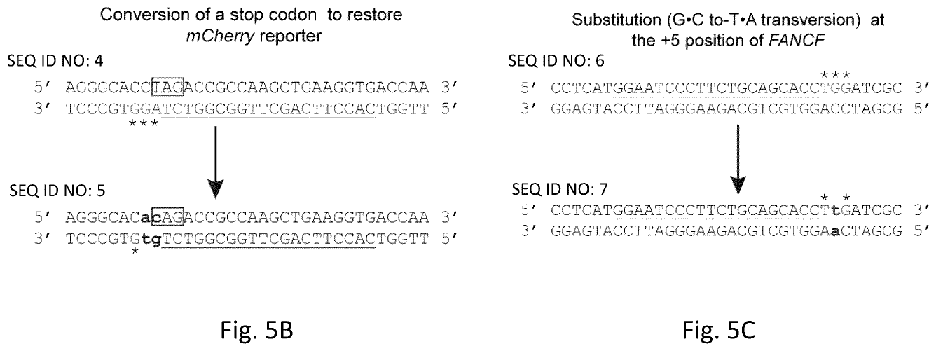

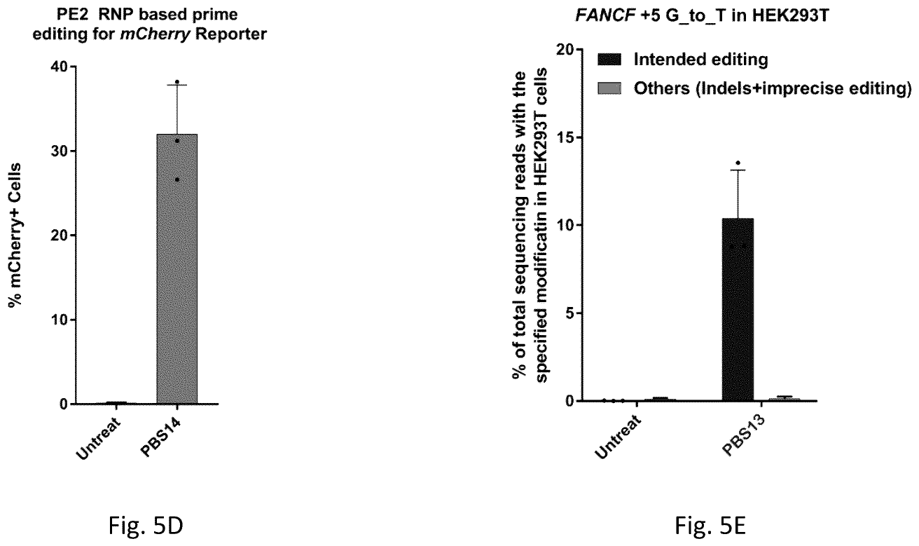

The bacterial expression and purification of prime editor (PE) protein has been described in the literature(21). We made modifications to the nuclear localization signal (NLS) sequences within the standard PE protein to improve its nuclear localization potential and included two additional point mutations from the PEmax architecture to improve the nickase activity(4, 6, 27). We then expressed and purified the PE protein from bacteria (FIG. 5A). We complexed the purified PE protein with synthetic, end-protected pegRNAs (PE RNPs) that were designed based on sequence composition parameters recommended by prior studies(1,29). However, initial tests of PE RNPs (PE2) delivered by electroporation to HEK293T cells yielded modest precise editing rates when employing pegRNAs with PBS lengths ˜13 nt (FIG. 5B-E). Previous studies using plasmid or lentiviral expression systems defined an optimal PBS length for the pegRNA of ˜13 nt in mammalian cells when the A·T and G·C distribution is relatively uniform(1,25). pegRNAs under those assay conditions were expressed endogenously via a U6 promoter and are subject to 3′ degradation(8). The PBS sequence is present at the 3′ end of the pegRNA, and so could be susceptible to truncation. We hypothesized that, in the case of chemically synthesized, end-protected pegRNAs, the PBS length requirements for optimal prime editing activity would be different from plasmid expressed pegRNAs. In particular, the optimal PBS length would reduce the complementarity between the spacer-PBS region to increase the rate of target recognition, nicking and RT priming.

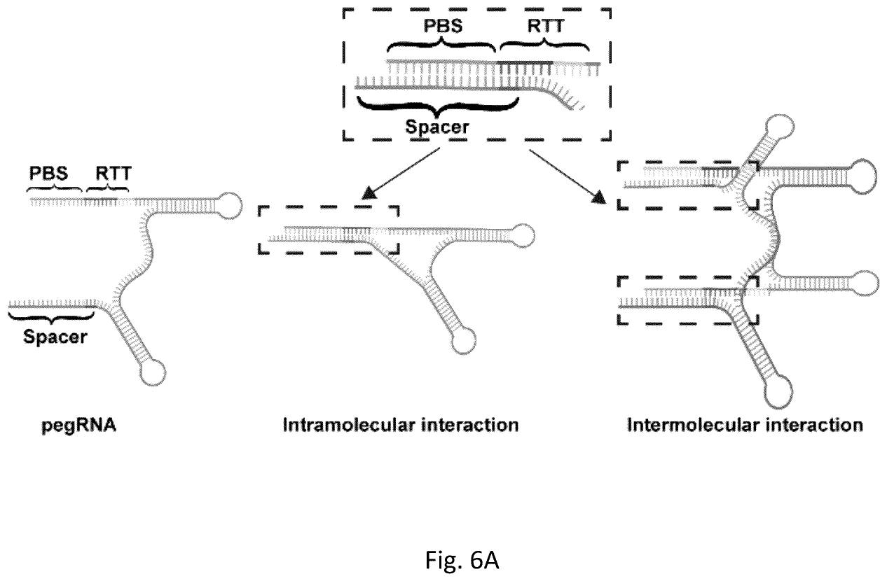

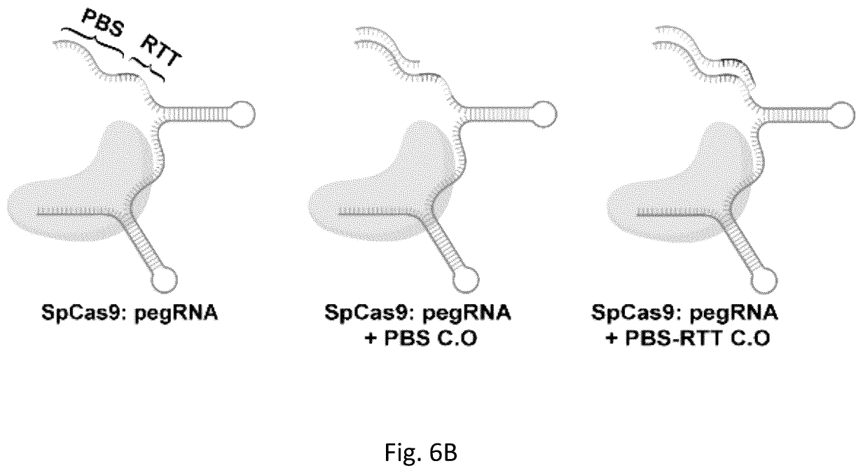

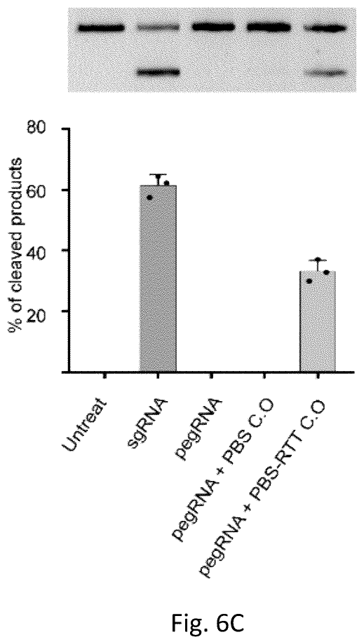

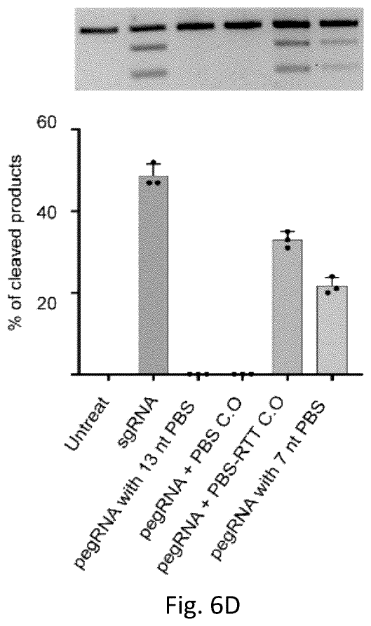

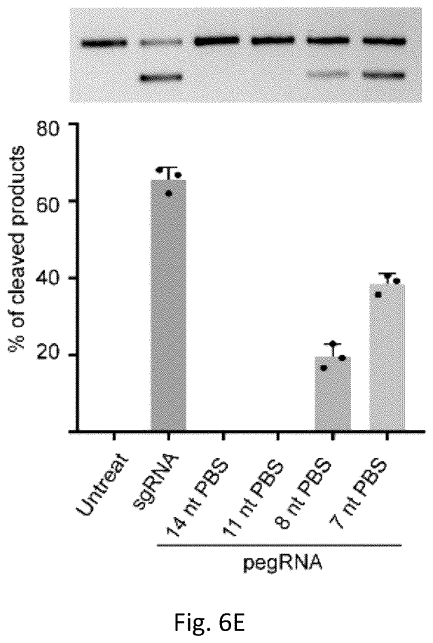

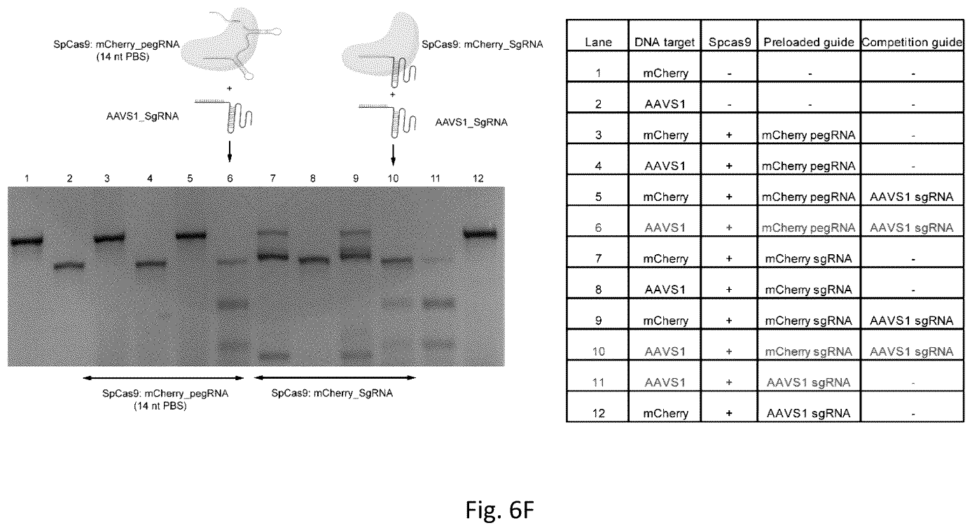

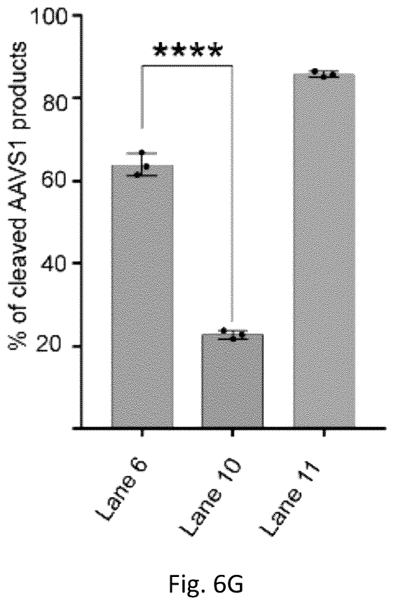

To test the level of auto-inhibition that is inherent in pegRNA structure (7) (FIG. 6A and FIG. 6B), we performed an in vitro DNA cleavage assay with SpCas9 nuclease complexed with synthetic pegRNAs. SpCas9 programmed with a pegRNA containing a standard ˜13 nt PBS was inactive for DNA cleavage (FIG. 6C and FIG. 6D). Inhibition was due to the PBS sequence, as co-administration of a competing oligonucleotide, complementary to the PBS-RTT region of the pegRNA, restored DNA cleavage activity (FIGS. 6B-D). Interestingly, including a competing oligonucleotide that is complementary only to the PBS region was not sufficient to overcome the auto-inhibition interaction at the concentration tested, which may be due in part to additional homology between the last three nucleotides of the RTT and spacer sequence. Thus, in vitro the cleavage activity of Cas9 can be restricted by the pegRNA sequence composition within the PBS region.

### Example 2: Synthetic pegRNAs with Shorter PBS Lengths Increase Prime Editing Efficiency of PE RNPs at Endogenous Loci

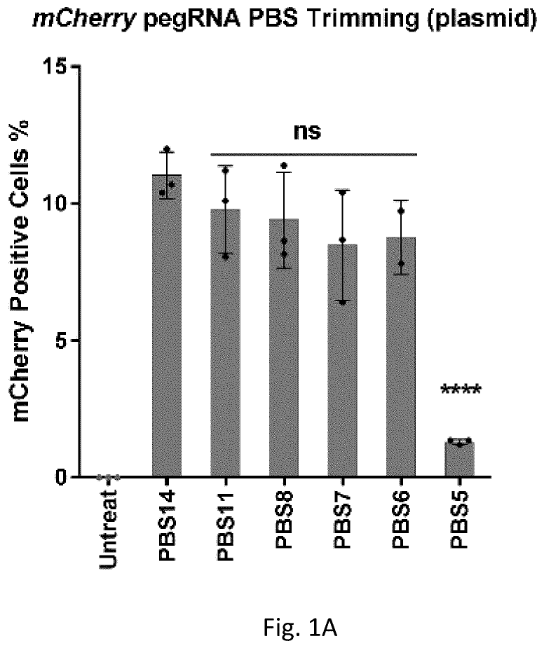

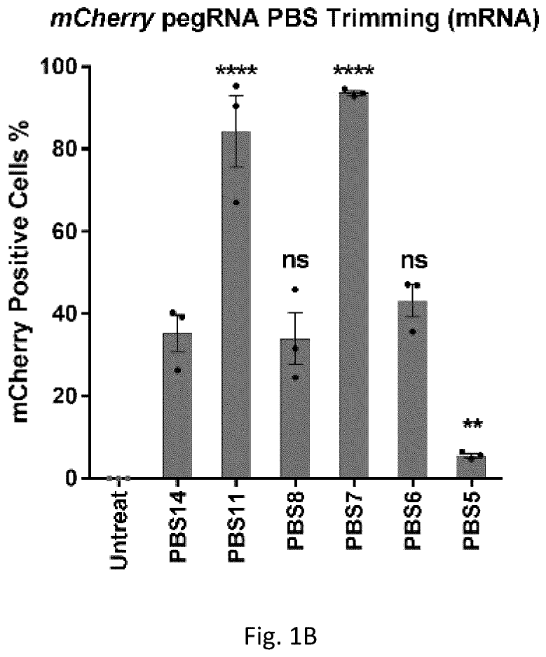

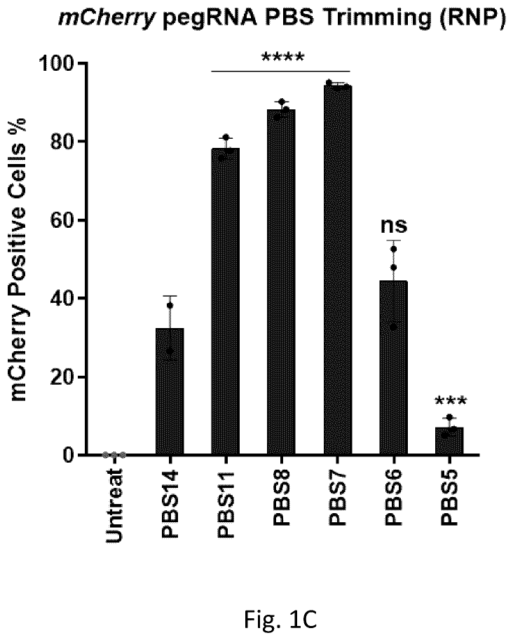

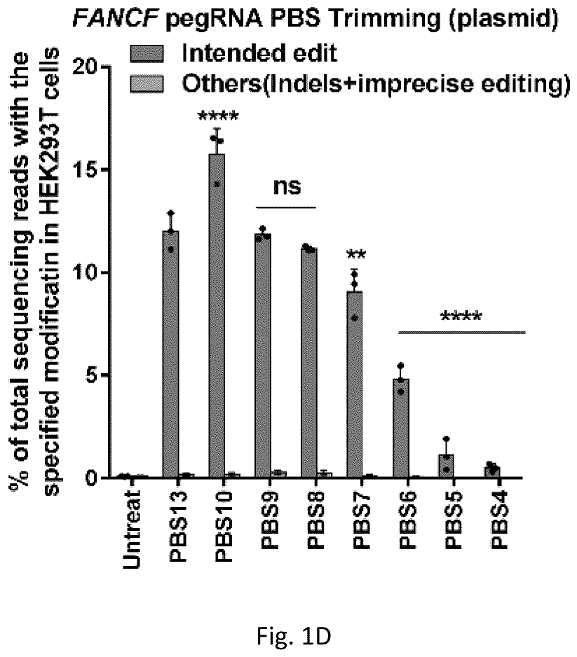

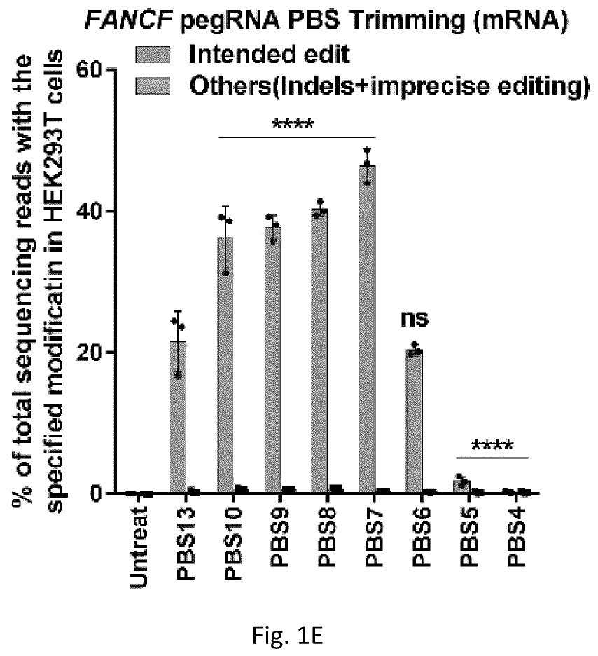

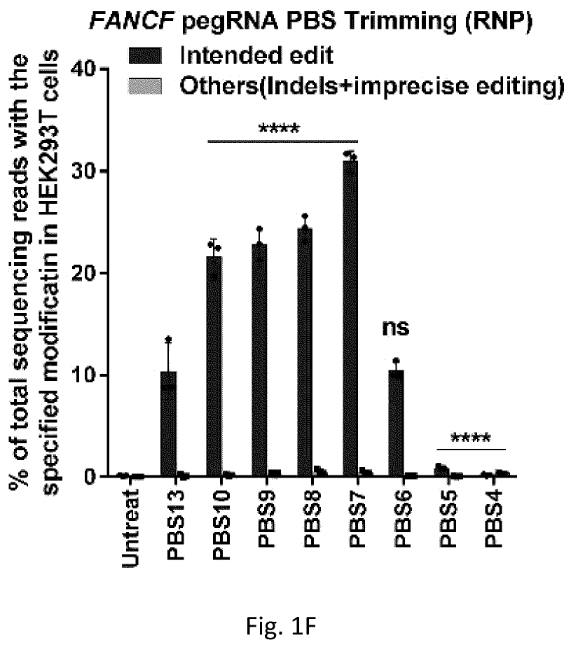

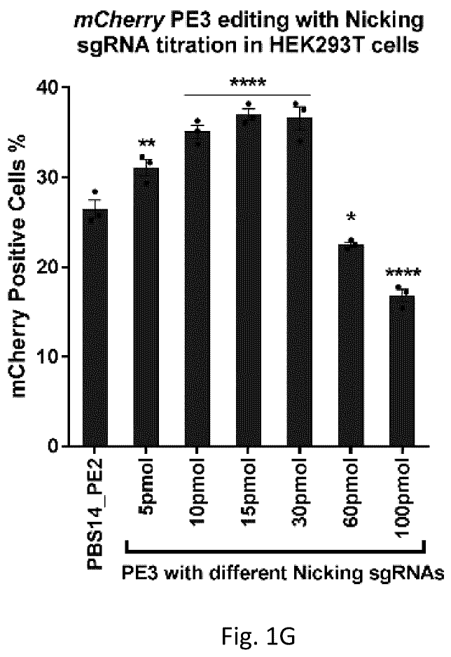

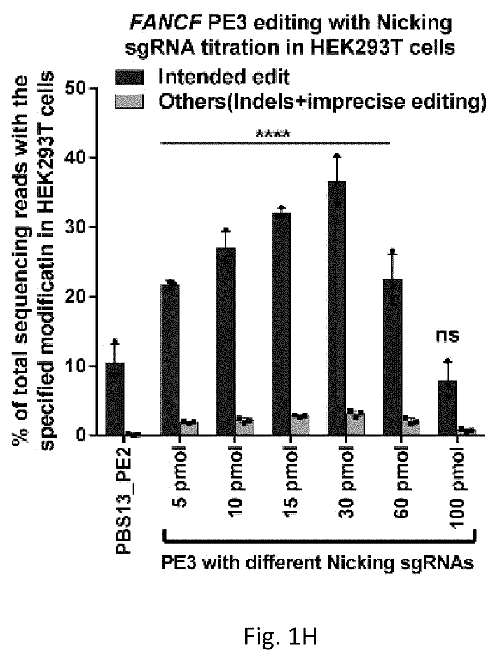

To examine the impact of the auto-inhibition interaction between the spacer and PBS sequence on PE activity, we tested the editing efficiency of a series of pegRNAs with different PBS lengths. We performed initial tests of these pegRNA designs in HEK293T cells on an mCherry reporter that contains a premature TAG stop codon that prevents translation of a functional protein(4) (FIG. 5C). We evaluated the prime editing efficiencies in the PE2 format for pegRNAs with different PBS lengths using three different delivery platforms (transfection of expression plasmids encoding the prime editor and pegRNA, or electroporation of PE mRNA or RNP with synthetic pegRNA). Consistent with prior studies for plasmid-encoded prime editor components, the 14 nt PBS had the highest editing efficiency (FIG. 1A). However, for PE mRNA or RNP delivered with synthetic pegRNAs, shorter PBS lengths provided higher activity, where the 7 nt PBS afforded the highest prime editing efficiency for both mRNA- and RNP-based systems (FIGS. 1B and 1C). Consistent with the increased prime editing rates observed when employing pegRNAs with a shorter PBS length, Cas9 nuclease activity in the in vitro DNA cleavage assay was also increased with these pegRNAs suggesting that auto-inhibition is reduced by the shorter PBS< > spacer complementarity (FIG. 6E).

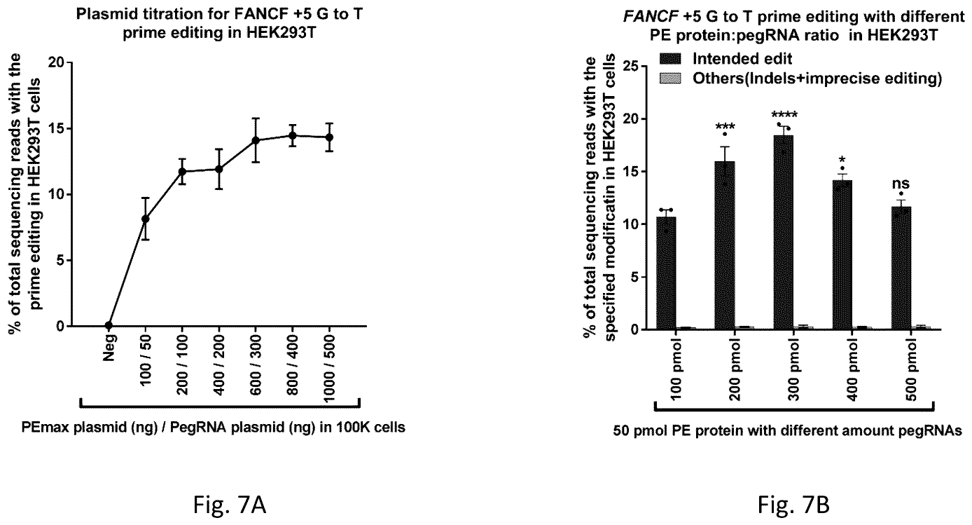

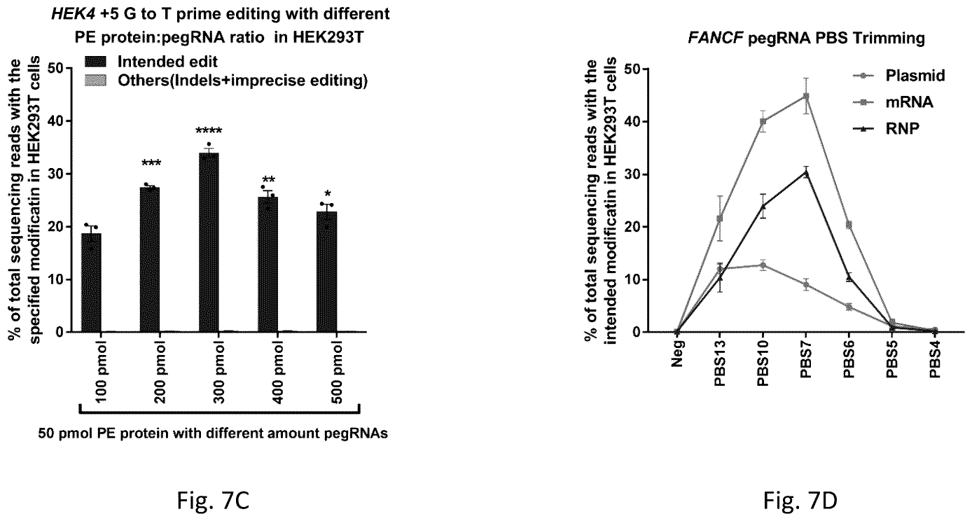

Motivated by our observations in the mCherry reporter cell line, we designed a series of pegRNAs with different PBS lengths for the previously described nucleotide substitution (+5 G to T) at the FANCF locus(1,21) (FIG. 5C). We observed the highest prime editing efficiency in the PE2 format when using a plasmid expression system under unsaturated conditions for the pegRNA with a 10 nt PBS (FIG. 1D, FIG. 7A), whereas the highest prime editing efficiency when delivering PE mRNA or RNP with a synthetic pegRNA occurred with a 7 nt PBS (FIG. 1E-F). Consistent with the prime editing activity outcome, the in vitro DNA cleavage assay using Cas9 nuclease programmed with the 7 nt PBS pegRNA targeting the FANCF site displayed higher activity (FIG. 6D).

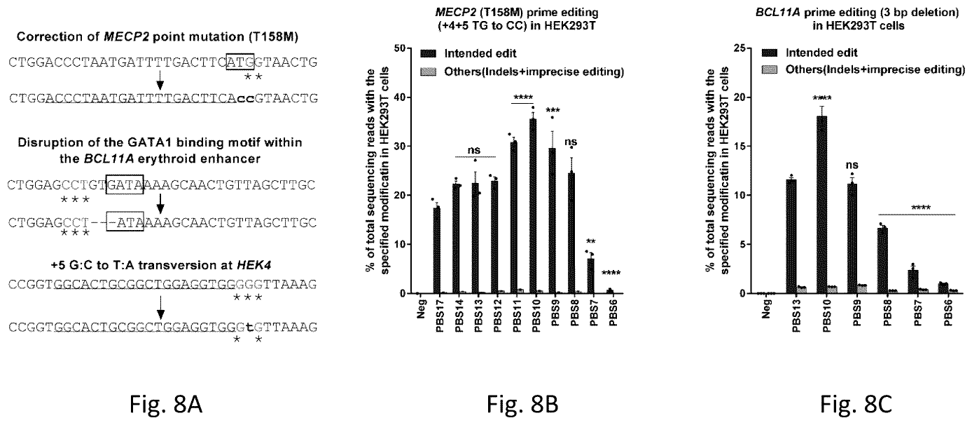

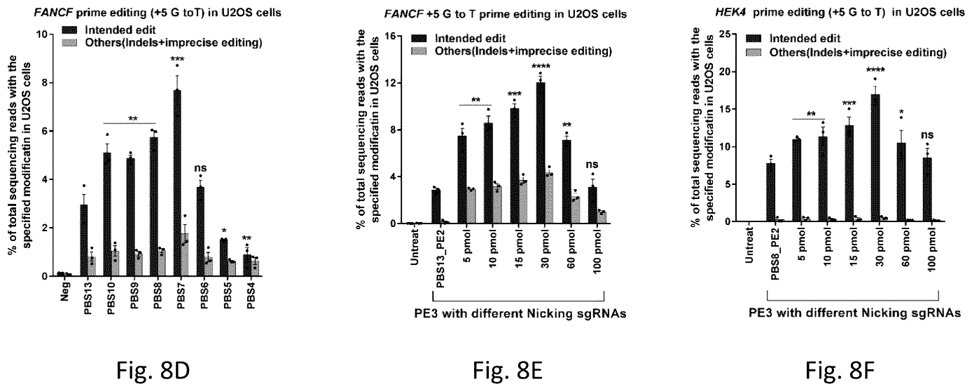

To determine if the observed trend for PBS length applies to other target site sequence compositions for PE RNPs, we evaluated the optimal PBS length for prime editing activity at two A/T-rich endogenous target sites, MECP2 and BCL11A. At MECP2 we used PE2 to correct a common point mutation (T158M) associated with Rett syndrome, an X-linked neurological disorder(30). The pegRNA PBS length series included a longer PBS (17 nt) based on the design parameters described by Anzalone et al., 2021 for A/T-rich target sites. Consistent with our prior evaluation of PE RNPs programmed with synthetic pegRNAs, shorter PBS lengths displayed higher rates of precise repair with a 10 nt PBS achieving maximum efficiency (FIG. 8A and FIG. 8B). At BCL11A we used PE2 to disrupt the GATA1 binding motif within the BCL11A erythroid enhancer that results in the induction of fetal γ-globin in erythroid progenitors and can ameliorate β-globinopathies like sickle cell disease and β-thalassemia(27,31). We designed pegRNAs with different PBS lengths designed to delete 3 bp from the GATA1 binding motif. Again, our results showed that for PE2-type RNPs, a pegRNA with a shorter PBS length (10 nt) creates the 3 bp deletion more efficiently than the pegRNA with a longer PBS length (FIG. 8A and FIG. 8C). Together, these results suggest that pegRNAs with shorter PBS lengths can broadly improve PE efficacy when employing an RNP format programmed with synthetic pegRNAs.

### Example 3: The Ratio of Nicking sgRNA and pegRNA Affects the Efficacy of PE3

Since we observed that the auto-inhibitory interaction between the PBS and the spacer sequence within the pegRNA interferes with target site cleavage, we questioned whether the spacer< >PBS interaction also impacts the binding affinity of Cas9 for the pegRNA. To test this, we designed an in vitro competition-based cleavage assay (FIG. 6F). We loaded Cas9 nuclease with an excess of either mCherry pegRNA or mCherry sgRNA for 20 minutes to form their respective RNP complexes. Next, a competing sgRNA targeting the AAVS1 locus was added to the binding reaction before carrying out the in vitro digestion with either the mCherry or AAVS1 target site for 20 minutes at 37° C. Since Cas9 cleavage of DNA in vitro is end-product inhibited(32), the amount of Cas9 complex loaded with each guide RNA can be assessed in the presence of excess DNA target. If the Cas9 nuclease has a lower binding affinity for the pegRNA compared to the sgRNA, the AAVS1 sgRNA should become preferentially bound to Cas9 even when preloaded with the mCherry pegRNA. When the mCherry sgRNA is pre-equilibrated with Cas9 and then competed with the AAVS1 sgRNA, the resulting complex has only modest cleavage activity on an AAVS1 PCR product. However, when the mCherry pegRNA is pre-equilibrated with Cas9 and then competed with the AAVS1 sgRNA, it cleaves the AAVS1 PCR product to a significantly greater extent (FIG. 5D and FIG. 5E). These data indicate that the binding affinity of Cas9 protein for the pegRNA is reduced relative to that of an sgRNA targeting the same locus.

Given the potential for the nicking sgRNA (Nk sgRNA) to displace the pegRNA in PE3 RNP editing applications if the pegRNA binds with lower affinity, we performed experiments to empirically determine the optimal ratio of the pegRNA and Nk sgRNA for maximal activity. We first performed an analysis for the optimal ratio of pegRNA to PE protein in the absence of Nk sgRNA at two different target sites (FANCF and HEK293T site 4 (HEK4)), which revealed higher precise editing rates as the pegRNA stoichiometry was increased up to a 6:1 ratio (FIG. 7B and FIG. 7C). We chose a 4:1 ratio of pegRNA to PE protein for a titration of Nk sgRNA to determine its optimal stoichiometry for PE3 editing. We tested PE3 editing in the mCherry reporter cell line (FIG. 1G) and at two endogenous target sites, FANCF and HEK4 in HEK293T cells (FIG. 1H, 1I). The PE protein was kept constant at 50 pmol and the pegRNA at 200 pmol. The Nk sgRNA concentration was varied from 5 pmol to 100 pmol. We observed that the PE3 system produced precise edits with the highest efficiency when a sub stoichiometric amount of Nk sgRNA is employed (15 to 30 μmol). At higher concentrations of the Nk sgRNA, the overall prime editing rate falls, which could be due to the sgRNA displacing the pegRNA across the majority of the delivered PE protein and thereby reducing the number of functional complexes for prime editing.

Recent studies have shown that mismatch repair (MMR) negatively influences prime editing outcomes(6,14). Given that HEK293T cells are partially MMR impaired(33), shifting prime editing to therapeutically relevant cell types that are proficient in MMR may reduce the rate of the desired editing outcome. To confirm that our PBS length analysis and optimal PE3 conditions for PE RNPs translate from HEK293T cells to other cell types where MMR is intact, we tested PE2 editing using the FANCF panel of pegRNAs with different PBS lengths (FIG. 8D) and PE3 editing at the FANCF and HEK4 loci in U20S cells(34) (FIG. 8E and FIG. 8F). We observed that prime editing outcomes in U20S cells for pegRNAs with different PBS lengths and different Nk sgRNA stoichiometries followed a similar trend as observed in HEK293T cells, albeit with lower precise editing rates.

### Example 4: Shorter PBS Lengths are Preferred for Plasmid Expression Systems that Generate 3′ End Protected epegRNAs

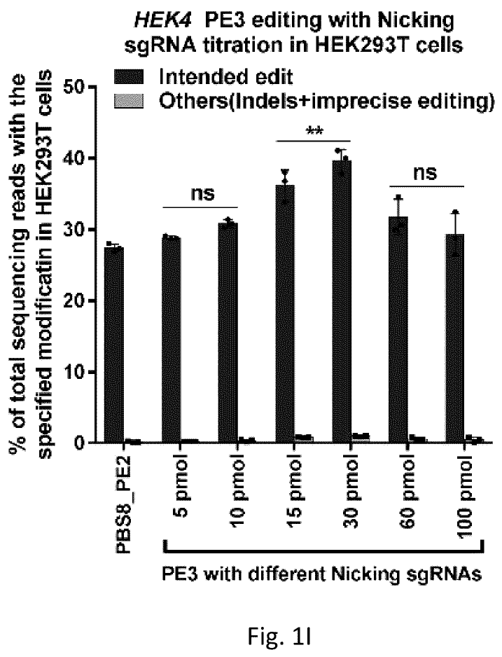

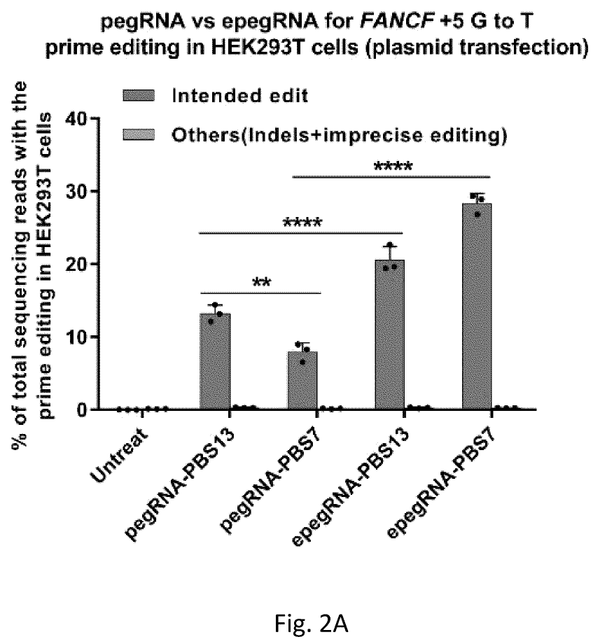

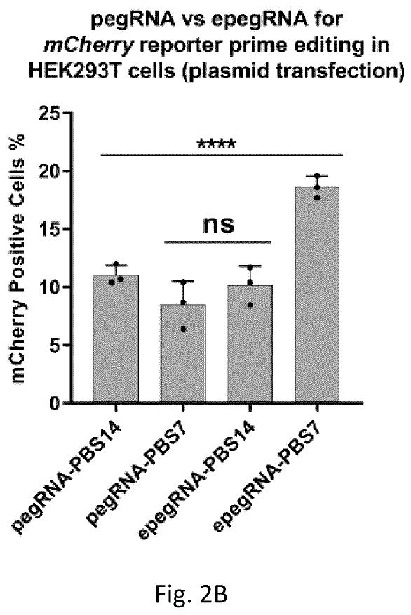

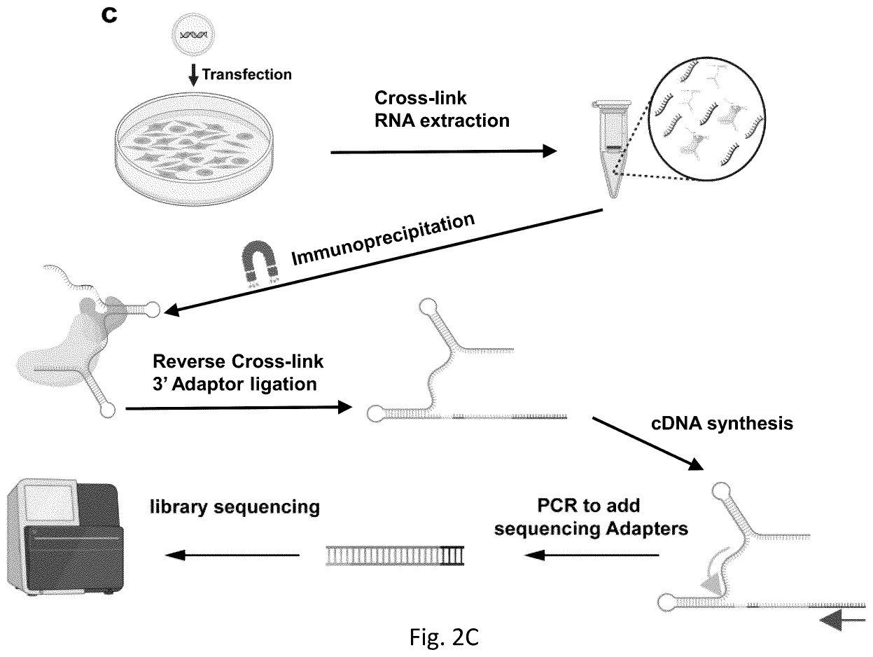

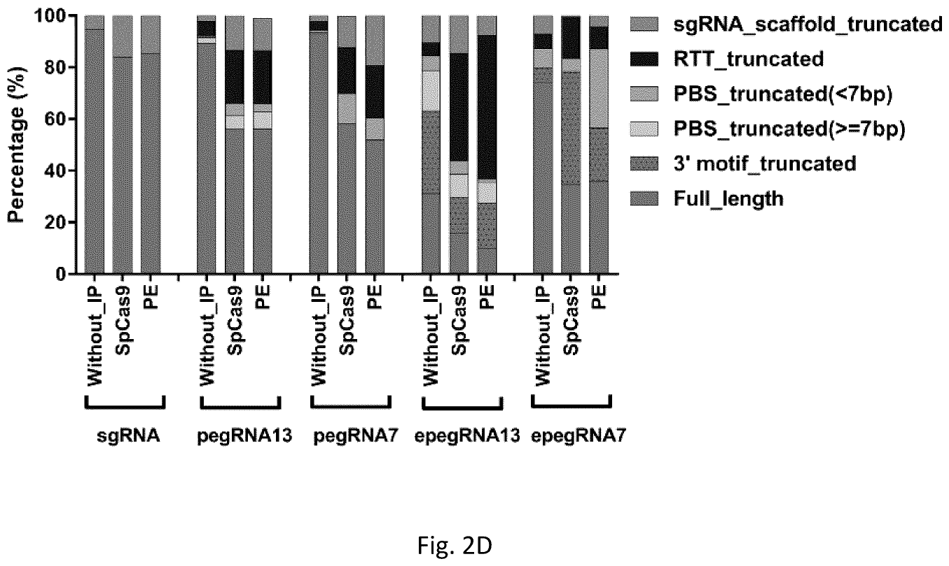

Based on our observation that the prime editor mRNA and RNP systems achieve higher rates of editing with shorter PBS lengths than plasmid expression systems (FIGS. 1A-I), we hypothesized that this dichotomy arises from the susceptibility of plasmid-expressed pegRNAs to 3′-exonuclease degradation(8). To address the 3′ degradation issue, others have appended a 3′ pseudoknot structure to stabilize the pegRNA sequence (referred to as an “epegRNA”), which increases the efficiency of prime editing(8). It has been demonstrated by Northern blot that although both pegRNAs and epegRNAs produced from plasmid expression systems are truncated in cells to varying degrees to a species of similar length to an sgRNA, epegRNAs are more stable than pegRNAs when exposed to cell lysates containing exonucleases. We hypothesized that the optimal PBS length would be shorter for epegRNAs produced from a plasmid expression system since they are 3′ end-protected similar to chemically synthesized pegRNAs. To explore the impact of PBS length on prime editing efficiency with epegRNAs, we built two epegRNA plasmid expression vectors for the FANCF target site (FANCF+5G→T), one with a 13 nt PBS and another with a 7 nt PBS. We observed higher precise editing rates for the epegRNA with the 7 nt PBS, which is consistent with the observations of prime editing with the chemically synthesized pegRNA at this site (FIG. 2A). Similarly, prime editing with an epegRNA containing a 7 nt PBS was superior to its longer PBS counterpart when targeting the stop codon in the mCherry reporter cell line (FIG. 2B). Thus, two different forms of pegRNA 3′ end-protection (chemical modification and RNA pseudoknot) yield similar changes in the optimal PBS length for prime editing.

### Example 5: 3′ Truncated Species Compete Full Length pegRNA for Loading onto Prime Editor Protein

The 3′ truncation of pegRNAs or epegRNAs expressed from plasmid could produce a distribution of species with different lengths. Based on our in vitro experiments evaluating the binding preference of Cas9 for an sgRNA over a pegRNA, different pegRNA truncation products could have different binding preferences to the prime editor protein when an excess of pegRNA is present within the cell. To examine the distribution of 3′ sequence lengths for U6 promoter-expressed pegRNA and epegRNA species and their relative loading distribution on prime editors, we performed small RNA-seq analysis on the total pegRNA and epegRNA population within the cell, and of the pegRNA and epegRNA bound to the immunoprecipitated prime editor protein (FIG. 2C). To eliminate the possibility that the RNaseH activity of MMLV-RT participates in the truncation of the pegRNA and epegRNA, we also performed pegRNA immunoprecipitation with Cas9 nuclease. Small RNA-seq on the bulk pegRNA and epegRNA species revealed that the majority of products were full-length or nearly full-length (FIG. 2D). However, we observed that the distribution of prime editor (or SpCas9) bound pegRNA and epegRNA species were enriched for truncated products. For the 13 nt PBS epegRNA, only ˜30% of the population loaded on the prime editor protein represents the full-length species, whereas for the 7 nt PBS epegRNA, ˜60-80% of the loaded population are full-length species (FIG. 2D). However, we do not notice a similar difference between the pegRNA with the 13 nt PBS and the pegRNA with the 7 nt PBS. In fact a greater fraction of bound truncated species was observed for the epegRNAs than the pegRNAs (FIG. 2D). This could be partly because an epegRNA has lower binding affinity than a pegRNA to Cas9(8). Regardless, it is evident that truncated species compete with full-length pegRNA for binding to the prime editor protein or Cas9 nuclease.

### Example 6: Tₘ of PBS:Spacer DNA Determines Optimal PBS Length

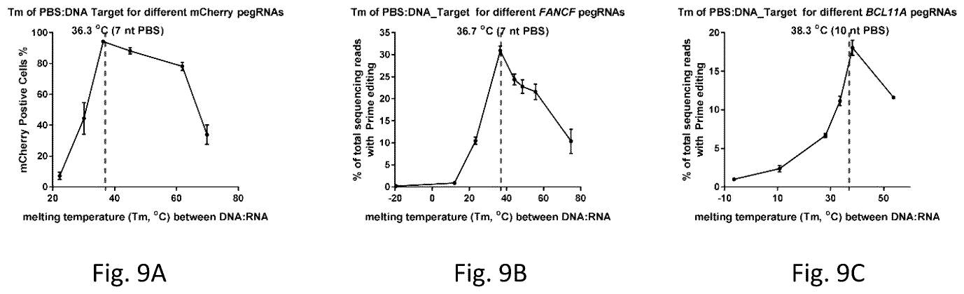

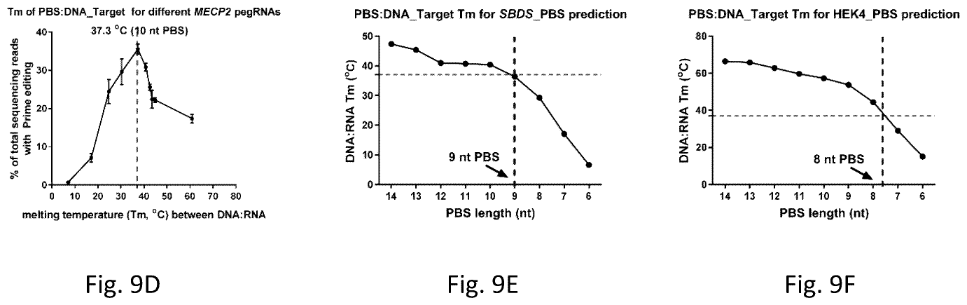

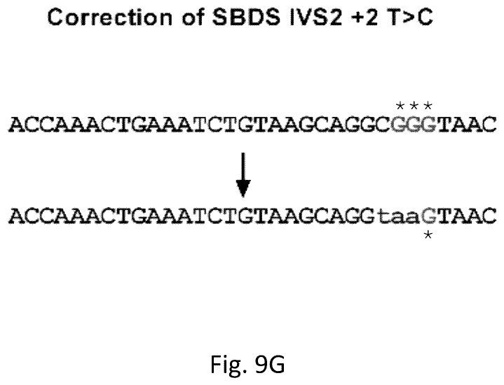

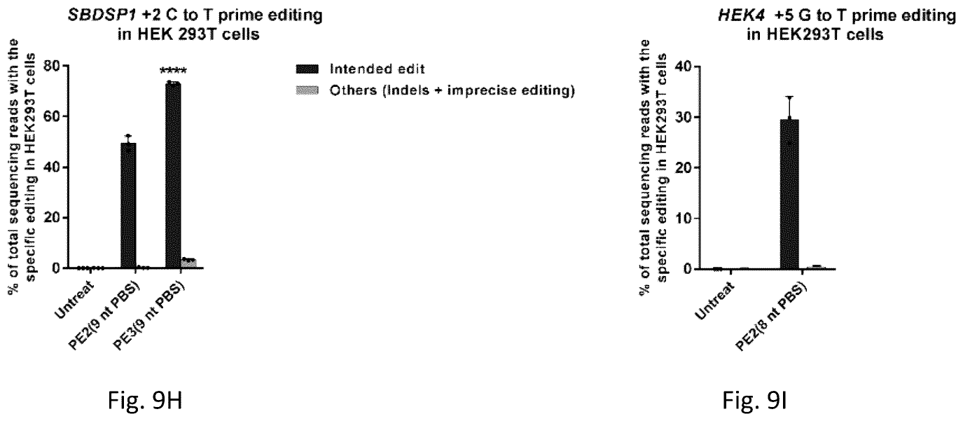

Consistent with prior models for PBS design(1,29), the optimal PBS length for precise editing was longer for the two A/T-rich target sites tested (MECP2 and BCL11A) than the two G/C-rich target sites (FANCF and mCherry). Using the MELTING 5 program(35), we estimated the melting temperature (Tm) of the optimal PBS sequence with the nicked target DNA for each pegRNA-target site combination. Surprisingly, we found that the estimated Tm of each PBS-target site combination for the optimal PBS length approaches 37° C., which is the growth temperature for mammalian cells (FIGS. 9A-D). To establish if we can design highly active pegRNAs based on the calculated PBS:target DNA Tm, we designed a pegRNA with a predicted Tm of −37° C. (9 nt PBS) for correction of SBDS IVS2+2T>C, a splice site mutation associated with almost all Shwachman-diamond syndrome cases(36) (FIG. 9E and FIG. 9G). This common mutation is believed to be derived via gene conversion from a neighboring pseudogene, SBDSP1(37)(38). Therefore, we tested the SBDS IVS2+2T>C correction pegRNA at the SBDSP1 site in HEK293T cells, which has an identical sequence with the SBDS IVS2+2T>C target site. We were able to achieve high editing rates up to 49.3% with PE2 and 73% with PE3 (FIG. 9H). Similarly, we designed a pegRNA with a predicted Tm of −37° C. (8 nt PBS) for the HEK4 target site (HEK4+5G→T) (FIG. 9F). We were able to achieve 29.7% editing rates with PE2 (FIG. 9I).

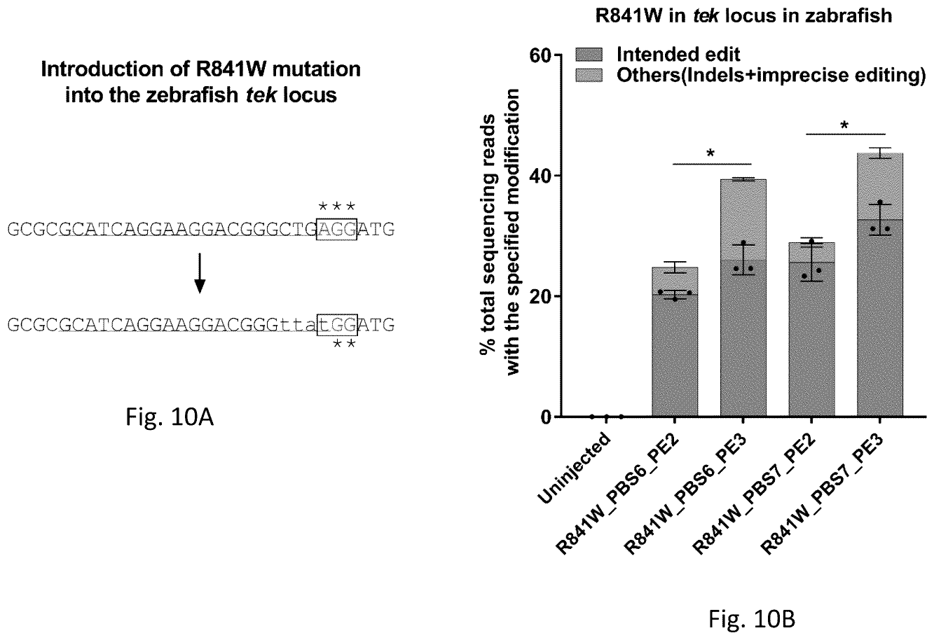

We also evaluated the utility of this PBS design parameter for prime editing in zebrafish embryos. PE RNPs have been used successfully to install germline mutations in zebrafish embryos with modest editing rates (<10%) for the introduction of point mutations(21). We focused on a mutation that leads to vascular malformations (VMs). VMs are associated with somatic and germline activating mutations in the gene encoding the endothelial-specific Angiopoietin-1 receptor tyrosine kinase, TEK(39,40). Germline mutations cause mild activation of the receptor and often require a somatic second hit to initiate VM formation. The most common germline mutation is an autosomal-dominant p.R849W change that leads to weak activation of the receptor(40). In zebrafish Tek, the homologous residue is R841. Zebrafish carrying the R841W mutations will provide a valuable tool for studying the cellular and molecular mechanisms of VMs during embryogenesis. Here, we designed a prime editing strategy to introduce the p.R841W mutation and a neighboring synonymous mutation into the zebrafish tek locus (FIG. 10A). We designed two pegRNAs that differ in the PBS length, one (7 nt) with a predicted Tm of 37° C. and another one (6 nt) with a predicted Tm near 28.5° C., which is the incubation temperature for zebrafish embryos. We observed that both these tek pegRNAs when delivered as PE2 RNPs to zebrafish embryos were able to efficiently introduce the desired codon conversions at the target site with an overall precise editing rate of ˜20 to 26% (FIG. 10B). Additionally, we tested if utilizing a PE3 approach would increase the rate of precise edits at the tek locus. We translated the pegRNA:sgRNA ratios that were optimized for the PE3 system in mammalian cells to zebrafish embryos. We saw a modest increase (˜1.2 fold) in precise editing rates when employing the PE3 approach compared to PE2, where we achieved an overall precise editing rate of 26 to 33% (FIG. 10B). Thus, optimizing the PBS length based on the reaction temperature for genome editing provides efficient editing outcomes in mammalian cells and zebrafish embryos.

### Example 7: Transient Cold Shock Enhances Prime Editor Activity

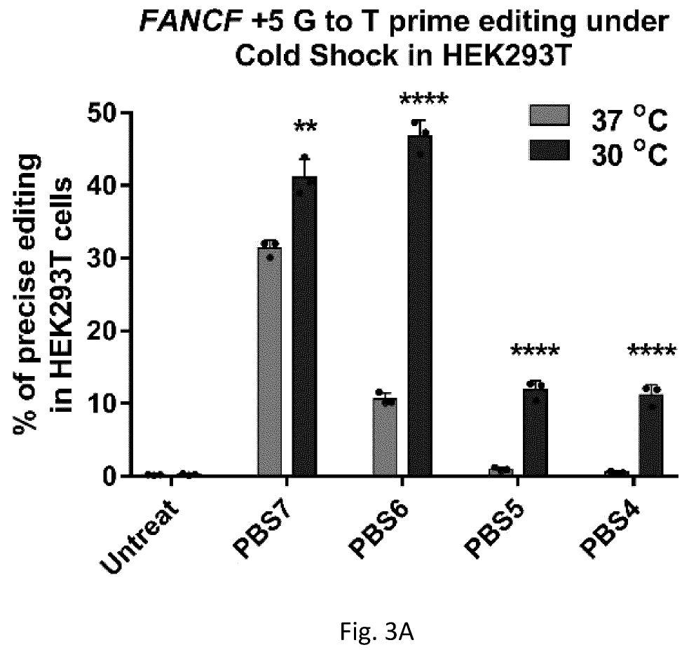

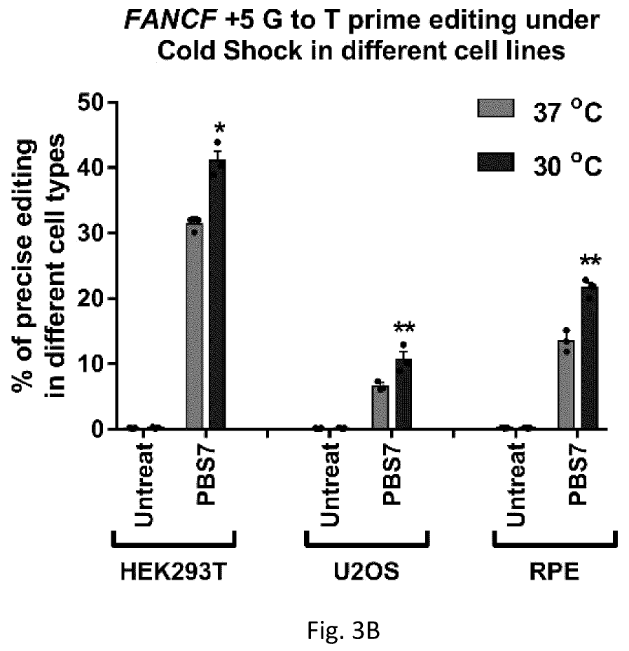

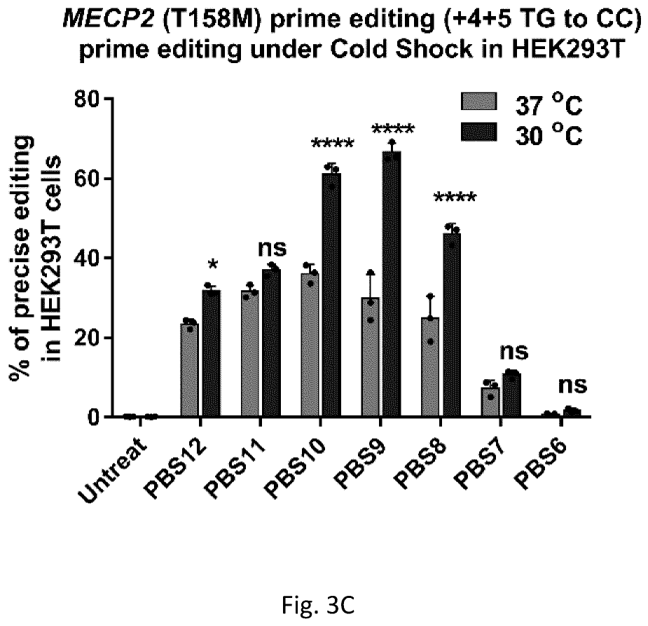

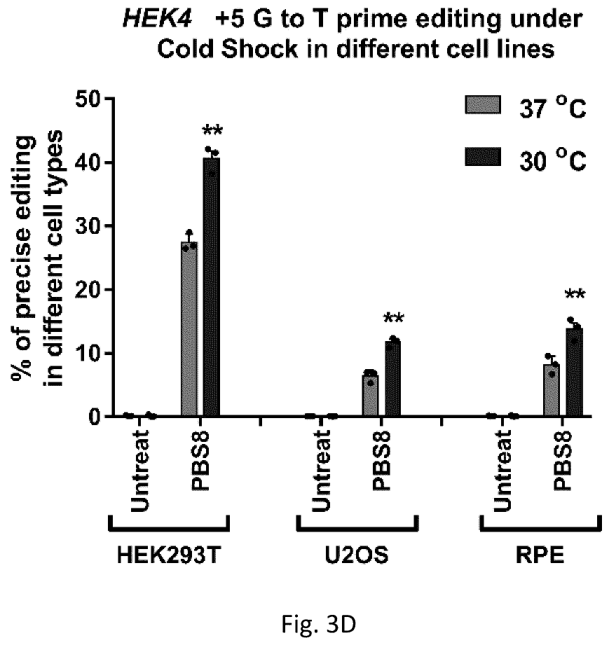

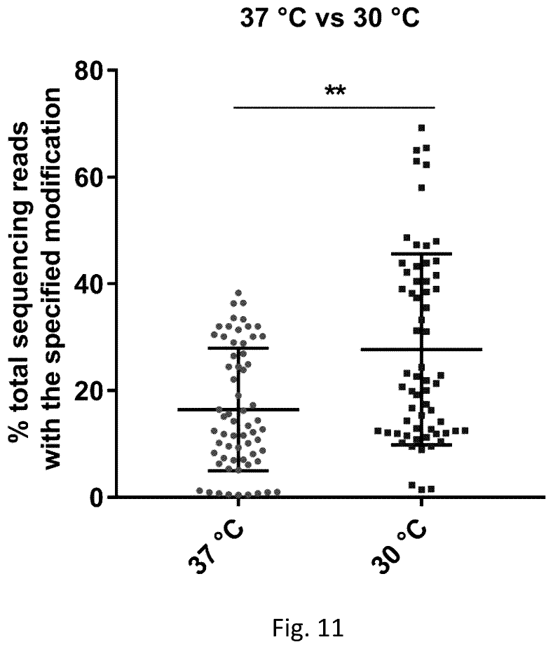

To further investigate if the PBS-target strand interaction is temperature dependent, we shifted the culture temperature of PE2 RNP treated cells post electroporation. We evaluated the prime editing efficiency of the FANCF pegRNA PBS panel in HEK293T, U20S and RPE-1 cells at 30° C. and 37° C. For the transient cold shock treatment, the cells were cultured at 30° C. overnight for 12-16 hrs post nucleofection and then transferred to 37° C. until the 72 hour editing analysis point. We quantified the editing efficiency using targeted amplicon deep sequencing. Consistent with the importance of the reaction temperature on the PBS length for efficient prime editing, we observed an increase in prime editing activity for pegRNAs with shorter PBS lengths at 30° C. relative to 37° C. (FIG. 3A). We also observed an unexpected increase in prime editing activity at 30° C. for the optimal PBS length (7 nt) compared to the standard 37° C. editing conditions. The observed increase in precise editing rates as a function of cold shock was independent of the cell type, where cold shock treatment increased the precise editing rates by 1.3 to 1.6 fold (FIG. 3B). We observed a similar increase in prime editing rates at 30° C. for pegRNAs targeting the HEK4 and MECP2 loci when using PE2 RNPs (FIG. 3C and FIG. 3D). FIG. 11 shows that cold shock treatment to the cells post PE RNP nucleofection significantly increases prime editing rates across multiple loci, different cell types, and different delivery methods (mRNA and RNP). Thus, subjecting cells to a cold shock post electroporation can alter the prime editing activity as a function of PBS length and modestly enhance prime editing efficiency in a variety of cell types.

### Example 8: Prime Editing in Patient-Derived Fibroblasts and Human Primary T Cells

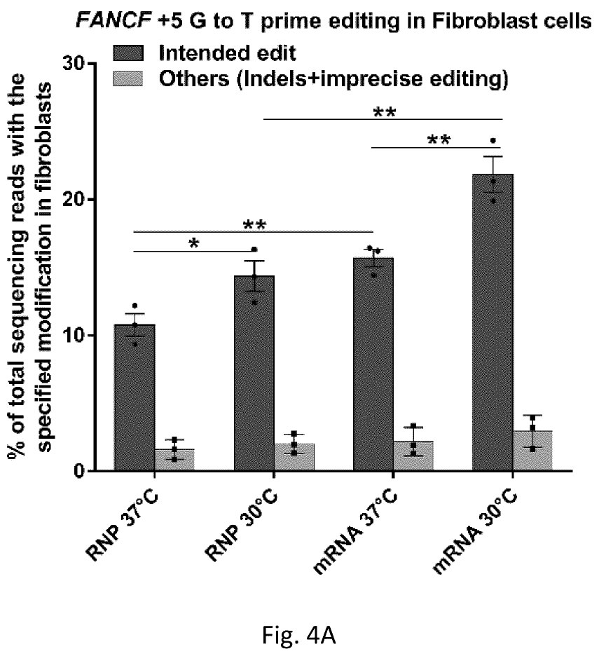

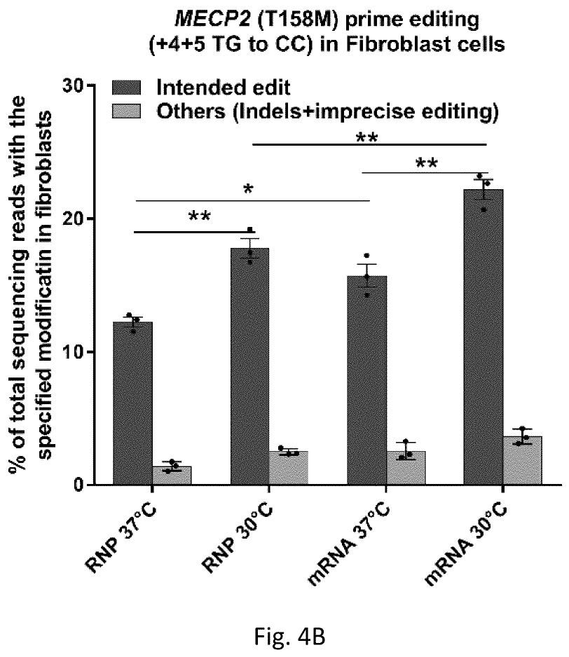

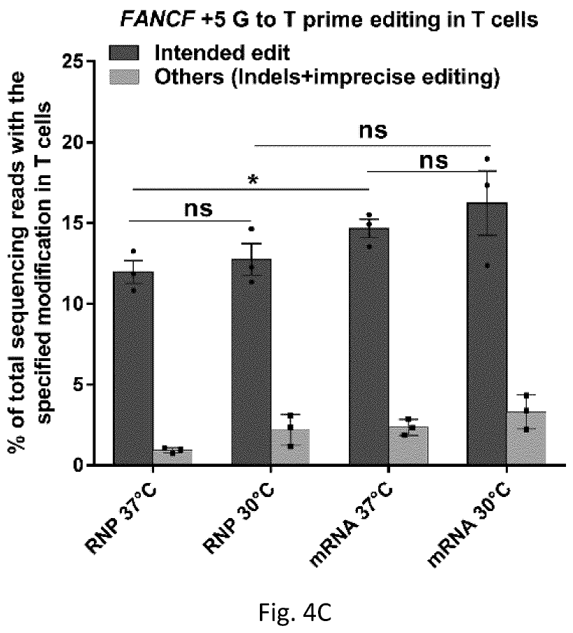

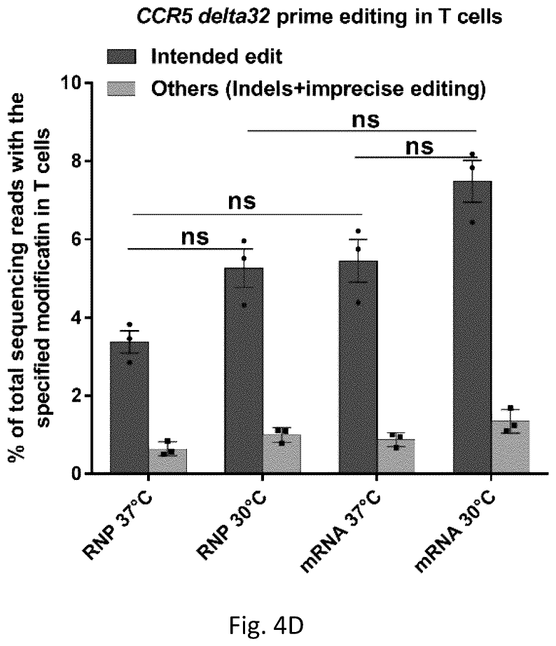

To demonstrate the therapeutic potential of PE RNPs using optimized PBS lengths, we tested prime editing in a Rett syndrome patient-derived fibroblast line that carries the T158M mutation, and in primary human T cells. In the Rett fibroblast line, we tested PE3 RNP or PE3 mRNA delivery targeting the FANCF (+5 G→T) and MECP2 T158M sites employing a pegRNA with a 7 nt PBS for FANCF or a 10 nt PBS for T158M. We observed 10.8% and 15.7%+5 G→T edits at the FANCF target site with RNP and mRNA respectively and 12.2% and 15.9% correction of the mutant allele at the MECP2 target site with RNP and mRNA, respectively (FIG. 4A and FIG. 4B). A transient cold shock treatment of these cells following electroporation further increased the prime editing efficiency by ˜1.5 fold at FANCF and MECP2 T158M for both PE3 RNP and PE3 mRNA delivery.

In primary human T cells, we tested PE3 RNP or mRNA delivery targeting the FANCF (+5 G→T) site evaluating editing at both 37° C. and with cold shock at 30° C. We observed 11.3% and 14.2% precise editing at 37° C. with PE3 RNP and PE3 mRNA respectively, which increased ˜1.2 fold with a cold shock treatment (FIG. 4C). We additionally designed a pegRNA to introduce the CCR5delta32 mutation into T cells, which is associated with HIV resistance(41). Using the MELTING 5 program(35), the optimal PBS length calculated for this pegRNA was 10 nt. Using a pegRNA with a 10 nt PBS with PE3 RNP or mRNA delivery by electroporation, we observed 3.4% and 5.1% rate of delta32 deletion with PE3 RNP and PE3 mRNA respectively when the T cells were grown at 37° C., and ˜1.4 fold increase in editing rates with a cold shock treatment (FIG. 4D).

### Materials and Methods

### General Methods and Molecular Cloning

To generate pegRNA expression plasmids, gblocks or PCR products including spacer sequences, scaffold sequences and 3′ extension sequences (RTT, PBS & pseudoknot) were amplified with indicated primers using Phusion master mix (ThermoFisher Scientific).

These amplicons were subsequently cloned into the sgRNA, pegRNA or epegRNA U6 expression vectors (Addgene, #122089) by the Gibson assembly method (NEB). To generate sgRNA expression plasmids, annealed oligos were cloned into BfuAI-digested vectors. To generate pegRNA and epegRNA expression plasmids, BfuAI and EcoRI digested vectors were used. All plasmids used for transfection experiments were purified using Midiprep kit including endotoxin removal step (ZymoPURE Plasmid Miniprep Kit from Zymo Research). pCMV-PEmax was a gift from David Liu (Addgene plasmid #174820). To generate PEmax protein expression vector (pET-21a-PEmax-6His), Primers were used to amplify the SpCas9-H840A and M-MLV ORFs from PEmax backbone, and then cloned into the bacterial expression plasmid pET-21a vector by Gibson assembly.

### Small RNA Sequencing

The immunoprecipitation protocol (IP) was adapted from the ChIP protocol described by the Castilo lab(24). HEK293T cells (10⁷ cells) were plated in 10 cm culture dishes and transfected with the prime editor components (10 μg of PEmax or Cas9 vector and 5 μg of pegRNA or epegRNA) using lipofectamine 3000 as per manufacturer's instructions. The cells were harvested and for the IP of effector-bound RNAs, cross-linked in 1% formaldehyde for 20 minutes at room temperature. The cells were then lysed using Pierce™ IP Lysis Buffer (Thermofisher scientific #87788). Immunoprecipitation of the Cas9 or PE RNP was carried out using anti-HA tag antibody—ChIP Grade (Abcam #ab9110) overnight at 4° C. Antibody bound RNP complexes were isolated using Dyna magnetic beads (Life technologies, 10004D). The immunoprecipitated RNP complex was then reverse cross-linked overnight at 65° C. DNAse (NEB, M0303S) and proteinase K (Thermofisher scientific, #25530049) treatment was carried out at 37° C. to remove the protein and DN-A. The RNA (pegRNA or epegRNA) was then purified using the Monarch® RNA Cleanup Kit (T2050L). The isolated RNA was then analyzed by deep sequencing. The small RNA library was built by a protocol adapted from the illumina TruSeq small RNA library protocol described by the Zamore lab(25).

In Vitro Transcription of PEmax mRNA Used in HEK293T, Fibroblast Cells and T Cell Experiments

PEmax coding region was cloned into an mRNA vector encoding an T7 promoter followed by a 5′ untranslated region (UTR), Kozak sequence, multiple cloning sites (MCS), and a 3′ UTR with a 125-nt poly(A) tail(26). Then the vector was linearized by the PmeI enzyme that cleaves after the polyA tail. PEmax mRNA was transcribed from 500 ng purified linearized template using the HiScribe T7 High-Yield RNA Synthesis Kit (New England BioLabs) with co-transcriptional capping by CleanCap AG (TriLink Biotechnologies) and full replacement of UTP with N1-Methylpseudouridine-5′-triphosphate (TriLink Biotechnologies). After 1 hour of in vitro transcription, the DNA template was digested by 1 μL DNase I (Thermo Fisher Scientific) for 15 min. Transcribed mRNAs were purified by RNA Clean & Concentrator-25 kit from Zymo Research, then purified mRNA was dissolved in nuclease-free water. The resulting PEmax mRNA was quantified with a NanoDrop One UV-Vis spectrophotometer (Thermo Fisher Scientific) and was stored at ˜80° C.

### PEmax Protein Purification

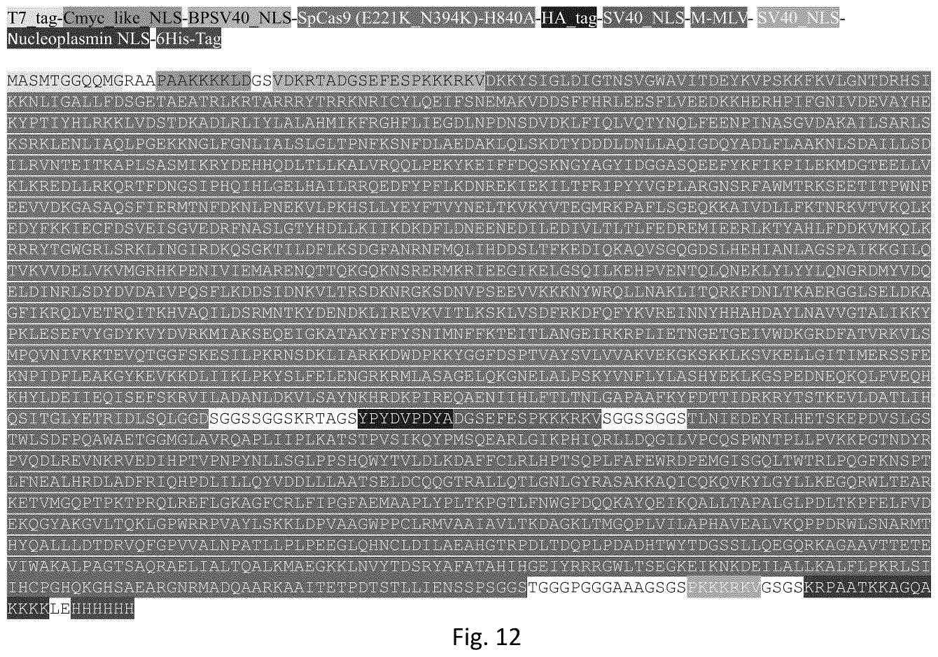

PEmax Protein purification protocol was adapted from a previously described protocol for 3×-NLS-SpCas9(27). pET-21a-PEmax-His₆ (FIG. 12) was introduced into E. coli Rosetta2(DE3)pLysS cells (EMD Millipore) for protein overexpression. Cells were grown at 37° C. to an OD600 of ˜0.6, then pre-chilled in an ice bath for 10 minutes and shifted to 18° C. At an OD600 of ˜0.8 the cells were induced for 16 hours with IPTG (0.7 mM final concentration). Following induction, cells were pelleted by centrifugation and then resuspended with Nickel-NTA buffer (20 mM TRIS+1 M NaCl+20 mM imidazole+1 mM TCEP, pH 7.5) supplemented with HALT Protease Inhibitor Cocktail, EDTA-Free (100×) [ThermoFisher] and lysed with LM-20 Microfluidizer (Microfluidics) following the manufacturer's instructions. The protein pellet was then purified with Ni-NTA resin in batch mode and eluted with elution buffer (20 mM TRIS, 500 mM NaCl, 250 mM Imidazole, 10% w/v glycerol, pH 7.5). The PEmax protein was dialyzed overnight at 4° C. in 20 mM HEPES, 500 mM NaCl, 1 mM EDTA, 10% w/v (8% v/v) glycerol, pH 7.5. Subsequently, The PEmax protein was step dialyzed from 500 mM NaCl to 200 mM NaCl (Final dialysis buffer: 20 mM HEPES, 200 mM NaCl, 1 mM EDTA, 10% w/v glycerol, pH 7.5). Next, the PEmax protein was purified by cation exchange chromatography (Column=5 ml HiTrap-S(Cytiva), Buffer A=20 mM HEPES pH 7.5+1 mM TCEP, Buffer B=20 mM HEPES pH 7.5+1 M NaCl+1 mM TCEP, Flow rate=5 ml/min, CV=column volume=5 ml). The primary prime editor protein peak was dialyzed into 20 mM HEPES pH 7.5, 300 mM NaCl and then concentrated to ˜30 uM.

### In Vitro Cleavage Assay Conditions

For the Cas9 nuclease based cleavage assay with pegRNA, 10 pmol of pegRNA or sgRNA was added to 5 μL of nuclease free water and then 5 pmol of Cas9 in its storage buffer (20 mM HEPES and 150 mM NaCl, pH 7.4) was added to this solution and incubated at room temperature for 20 minutes for the RNP complex formation. For reactions with competing oligonucleotide, the pegRNA and the competing oligo (50 μmol) were heated together to 95° C. and allowed to cool at room temperature for 5 minutes before complexing with Cas9 nuclease as described above. Following RNP complex formation, 2 μL of NEB cutsmart buffer and 500 ng of PCR product containing the target sequence was added to the Cas9 RNP. Finally, nuclease free water was added to the reaction to bring the total reaction volume to 20 μL. The cleavage reaction was then incubated at 37° C. for 20 minutes followed by proteinase K treatment for 10 minutes to stop the cleavage reaction and to digest away the Cas9 that is bound to the DNA ends. The reaction was then run on a 2% agarose gel to observe the cleaved products. To examine the relative binding affinity of Cas9 for a pegRNA or sgRNA, we set up an in vitro competition-based cleavage assay. Here we first load 5 pmol of Cas9 nuclease with either 10 pmol of mCherry pegRNA or 10 pmol mCherry sgRNA. After allowing the RNP complex to equilibrate at room temperature for 20 minutes, we add 10 pmol of the competing sgRNA, AAVS1, and carry out the in vitro digestion of the appropriate PCR product under the same buffer conditions and temperature as described above.

### Culture Conditions for Immortalized Cell Lines and Patient Derived Fibroblasts

HEK293T cells and U2OS cells were purchased from ATCC. RPE-1 cells were a gift from the Sharon Cantor lab. A HEK293T based cell line that contains the MECP2 editing locus with some common Rett syndrome mutations was constructed as described in our recent work (manuscript currently under review). Patient derived fibroblasts containing the T158M mutation were obtained from the Rett Syndrome Research Trust. All cells were maintained in Dulbecco's Modified Eagle's Medium supplemented with 10% FBS at 37° C. and 5% CO₂ unless otherwise noted.

### Transfection of HEK293T and U20S Cells

To define unsaturated prime edit conditions for comparison of the activity of various pegRNAs, a series of prime editing reactions were tested where the amount of PEmax plasmid (100 ng, 200 ng, 400 ng, 600 ng, 800 ng, 1000 ng) and pegRNA plasmid (50 ng, 100 ng, 200 ng, 300 ng, 400 ng, 500 ng) were delivered by transfection to HEK293T cells keeping the ratio of PEmax:pegRNA at 2:1 (FIG. 7A). A ratio of 200 ng PEmax plasmid to 100 ng pegRNA was chosen for editing activity comparisons. For transfection-based editing experiments, HEK293T and U20S cells were plated 40,000 cells per well in a 48-well plate. 24 hours later, the cells were co-transfected with 200 ng of prime editor plasmid, 100 ng of pegRNA plasmid. Lipofectamine 3000 (Invitrogen) was used for the transfection according to the manufacturer's instructions. To determine editing rates at endogenous genomic loci, cells were cultured 3 days following transfection, after which the media was removed, the cells were harvested, and genomic DNA was isolated using QIAamp DNA mini kit (QIAGEN) according to the manufacturer's instructions. The editing rates were then determined by targeted amplicon deep sequencing or by a flow cytometer in the case of the mcherry reporter line.

### Electroporation of HEK293T, U2OS, RPE-1, and Fibroblast Cells

PEmax mRNA-sgRNA mixtures or RNPs were delivered by electroporation using the NEON Nucleofection System 10 μL kit (Thermo Fisher Scientific). For PEmax mRNA based editing experiments, 100k cells were pelleted at 300 g for 5 min and resuspended in 9 μL NEON Buffer R. The cell solution was combined with a 3 μL mixture of 1 μg PEmax mRNA, 100 pmol synthetic pegRNA (IDT) and 15 pmol synthetic sgRNA in R buffer from the NEON nucleofection kit (Invitrogen). The NEON Nucleofection System (Invitrogen) was used for electroporation with 10 μL tips (HEK293T: 1150v, 20 ms, 2 pulses; U20S: 1200v, 20 ms, 2 pulses; RPE-L: 1350v, 20 ms, 2 pulses and fibroblasts: 1200v, 30 ms, 2 pluses). For RNP based editing experiments, 50 pmol of PEmax protein was incubated with 200 pmol of pegRNA and 15 pmol of nicking guide RNA (150 pmol of PE protein with 600 pmol of pegRNA and 45 pmol of nk sgRNA were used in case of fibroblasts) in R buffer to a total volume of 10 μL for 15 min at room temperature. Then 100k cells were electroporated with 10 μL of PEmax RNP complex using the same electroporation conditions described above for mRNA nucleofection. gDNA was isolated 3 days after electroporation from each group and stored at −80 for Illumina library preparation.

### Prime Editing Experiments in Human Primary CD4 T Cells

To generate primary CD4+ T cells, peripheral blood mononuclear cells (PBMCs) were isolated from human donor leukopaks (source) by gradient centrifugation on lymphoprep (cat #07861, Stemcell Technologies). Thereafter, PBMCs were depleted of CD14 mononuclear cells using anti-CD14 microbead antibodies (cat #130-050-201, Miltenyi Biotec) and the flowthrough was enriched for CD4+ T cells by positive selection using anti-CD4 microbead antibodies (cat #130-045-101, Miltenyi Biotec). CD4+ T cell enrichment was confirmed by determining the percentage of CD3+/CD4+ cells via flow cytometry. Isolated CD4+ T cells were cultured in complete RPMI-IL2 media (RPMI-1640 media (cat #11875093, Thermofisher Scientific) supplemented with 10% heat-inactivated Cosmic Calf Serum (cat #SH30087.03, GE lifesciences), 25 mM HEPES pH 7.2 (cat #25-060-CI, Corning), 20 mM GlutaMAX (cat #3505-061, Gibco), 1 mM Sodium pyruvate (cat #25-000-CI, Corning), 1×MEM non-essential amino acids (cat #25-025-CI, Corning), 1% penicillin-streptomycin(cat #15140-122, Gibco), and 1:2000 human interleukin-2 (made in-house from IL-2 expressing cell line). 3 days prior to electroporation, primary CD4+ T cells were activated with anti-CD3/CD28 antibodies (cat #10971, Stemcell Technologies). 150 pmol of PE protein with 600 pmol of pegRNA (IDT) and 45 pmol of NK sgRNA (IDT) were used for RNP complex formation. 1e6 activated primary CD4+ T cells were electroporated with prime editing RNPs using the P3 primary cell nucleofector kit (cat #V4XP-3032, Lonza Biosciences) and program EH-115 on an Amaxa 4D-Nucleofector. The CD4+ T cells were allowed to recover for 72 hours at 37° C. with or without cold shock before genomic DNA is extracted using the Qiagen QiAamp DNA Blood Mini Kit (cat #51104, Qiagen).

### Cold Shock Treatment for Cells Post-Electroporation

Post nucleofection of the PE mRNA or PE RNP, the cells were moved to an incubator set at 30° C. and 5% CO₂ for 12-16 hours. After which, the cells were moved back to 37° C. and 5% CO₂. 72 hours post nucleofection, genomic DNA was harvested from the cells using the Qiagen DNeasy Blood and Tissue kit (Qiagen).

### Zebrafish Prime Editing Experiments

Zebrafish were maintained and bred according to standard protocols set by University of Massachusetts Chan Medical School Institutional Animal Care and Use Committee. Zebrafish embryos obtained from EK (WT) wild-type in-crosses were used for one cell-stage microinjections of PE RNPs. Prior to injections the tek target sequence was verified by Sanger sequencing. For PE2, 12 μM pegRNA (synthesized by IDT) and 6 μM PE protein were combined in nuclease-free water. For PE3 a nicking sgRNA (synthesized by IDT) was added to the PE2 complex at a 1 to 10 nicking sgRNA to pegRNA molar ratio. Complexes were incubated at room temperature for 5 minutes and then 2 nl was injected into single-cell embryos. Injected embryos were incubated at 28.5° C. overnight. Twenty-four hours post injection embryos were assessed for toxicity and genomic DNA was extracted from 20 normally developing embryos using the Qiagen DNeasy Blood and Tissue kit (Qiagen). Injections were performed in three independent replicates.

### Targeted Amplicon Deep Sequencing to Assess Editing Rates

Genomic DNA was isolated for prime editing analysis from treated cells or zebrafish embryos. Genomic loci spanning each target site were PCR amplified with locus-specific primers carrying tails complementary to the Truseq adapters. 200 ng of genomic DNA was used for the 1^{st} PCR using Phusion master mix (Thermo) with locus specific primers that contain tails. PCR products from the 1^{st} PCR were used for the 2^{nd} PCR with i5 primers and i7 primers to complete the adaptors and include the i5 and i7 indices. PCR products were purified with Ampure beads (0.9×reaction volume) and eluted with 25 ul of TE buffer, and were quantified by Qubit. Equal molar ratios of each amplicon were pooled and sequenced using Illumina Miniseq. Amplicon sequencing data was analyzed with CRISPResso (https://crispresso.pinellolab.partners.org/) (28). Briefly, demultiplexing and base calling were both performed using bcl2fastq Conversion Software v2.18 (Illumina, Inc.), allowing 0 barcode mismatches with a minimum trimmed read length of 75. Alignment of sequencing reads to each amplicon sequence was performed using CRISPResso2 in standard mode using the parameters “-q 30”. For each amplicon, the CRISPResso2 quantification window was positioned to include the entire sequence between pegRNA- and Nk sgRNA-directed Cas9 cut sites, as well as an additional 10 bp beyond both cut sites. For quantification of PE activity at the target site, editing efficiency was calculated as the percentage of reads with the desired edit without indels (“-discard_indel_reads TRUE.” mode) out of the total number of reads ((number of desired edit-containing reads)/(number of reference-aligned reads)). For all experiments, indel frequency was calculated as the number of discarded reads divided by the total number of reads ((number of indel-containing reads)/(number of reference-aligned reads)). The editing rate should be the number of reads containing indels out of the total number of reads.

### Statistical Analyses

Statistical analyses for plotted data were performed using GraphPad Prism 8.4. In all studies, data represent biological replicates (n) and are depicted as mean±s.d. as indicated in the figure legends. Comparison of mean values was conducted with unpaired, two-tailed Student's t-test; one-way ANOVA; or two-way ANOVA with Tukey's multiple comparisons test, as indicated in the figure legends. In all analyses, P values <0.05 were considered statistically significant.

### Data Availability/Sequence Data Resources

Illumina Sequencing data have been submitted to the Sequence Read Archive. These datasets are available under BioProject Accession number PRJNA907921 (www.ncbi.nlm.nih.gov/bioproject/?term=PRJNA907921) (SRA number: SRR23012416˜SRR23012421). Backbone plasmids used for pegRNA and sgRNA cloning are available from Addgene (#122089). The PEmax protein expression vector will be deposited with Addgene.

### Web Sites/Data Base Referencing/Programs

CRISPResso (github.com/pinellolab/CRISPResso2)
BWA (v0.7.17) (github.com/lh3/bwa)
Samtools (v1.16.1) (github.com/samtools/samtools)
umi_tools (vi.1.2) (umi-tools.readthedocs.io/en/latest/)
Aravind J, Krishna G K (2022). rmelting: R Interface to MELTING 5. R package version 1.14.0 (aravind-j.github.io/rmelting/)
GraphPad Prism 8.4

### REFERENCES

1. Anzalone, A. V., Randolph, P. B., Davis, J. R., Sousa, A. A., Koblan, L. W., Levy, J. M., Chen, P. J., Wilson, C., Newby, G. A., Raguram, A. et al. (2019) Search-and-replace genome editing without double-strand breaks or donor DNA. Nature, 576, 149-157.
2. Rees, H. A. and Liu, D. R. (2018) Base editing: precision chemistry on the genome and transcriptome of living cells. Nat Rev Genet, 19, 770-788.
3. Park, S. J., Jeong, T. Y., Shin, S. K., Yoon, D. E., Lim, S. Y., Kim, S. P., Choi, J., Lee, H., Hong, J. I., Ahn, J. et al. (2021) Targeted mutagenesis in mouse cells and embryos using an enhanced prime editor. Genome Biol, 22, 170.
4. Liu, P., Liang, S. Q., Zheng, C., Mintzer, E., Zhao, Y. G., Ponnienselvan, K., Mir, A., Sontheimer, E. J., Gao, G., Flotte, T. R. et al. (2021) Improved prime editors enable pathogenic allele correction and cancer modelling in adult mice. Nat Commun, 12, 2121.
5. Song, M., Lim, J. M., Min, S., Oh, J. S., Kim, D. Y., Woo, J. S., Nishimasu, H., Cho, S. R., Yoon, S. and Kim, H. H. (2021) Generation of a more efficient prime editor 2 by addition of the Rad51 DNA-binding domain. Nat Commun, 12, 5617.
6. Chen, P. J., Hussmann, J. A., Yan, J., Knipping, F., Ravisankar, P., Chen, P. F., Chen, C., Nelson, J. W., Newby, G. A., Sahin, M. et al. (2021) Enhanced prime editing systems by manipulating cellular determinants of editing outcomes. Cell, 184, 5635-5652 e5629.
7. Liu, Y., Yang, G., Huang, S., Li, X., Wang, X., Li, G., Chi, T., Chen, Y., Huang, X. and Wang, X. (2021) Enhancing prime editing by Csy4-mediated processing of pegRNA. Cell Res, 31, 1134-1136.
8. Nelson, J. W., Randolph, P. B., Shen, S. P., Everette, K. A., Chen, P. J., Anzalone, A. V., An, M., Newby, G. A., Chen, J. C., Hsu, A. et al. (2022) Engineered pegRNAs improve prime editing efficiency. Nat Biotechnol, 40, 402-410.
9. Li, X., Zhou, L., Gao, B. Q., Li, G., Wang, X., Wang, Y., Wei, J., Han, W., Wang, Z., Li, J. et al. (2022) Highly efficient prime editing by introducing same-sense mutations in pegRNA or stabilizing its structure. Nat Commun, 13, 1669.
10. Lin, Q., Jin, S., Zong, Y., Yu, H., Zhu, Z., Liu, G., Kou, L., Wang, Y., Qiu, J. L., Li, J. et al. (2021) High-efficiency prime editing with optimized, paired pegRNAs in plants. Nat Biotechnol, 39, 923-927.
11. Choi, J., Chen, W., Suiter, C. C., Lee, C., Chardon, F. M., Yang, W., Leith, A., Daza, R. M., Martin, B. and Shendure, J. (2022) Precise genomic deletions using paired prime editing. Nat Biotechnol, 40, 218-226.
12. Anzalone, A. V., Gao, X. D., Podracky, C. J., Nelson, A. T., Koblan, L. W., Raguram, A., Levy, J. M., Mercer, J. A. M. and Liu, D. R. (2022) Programmable deletion, replacement, integration and inversion of large DNA sequences with twin prime editing. Nat Biotechnol, 40, 731-740.
13. Wang, J., He, Z., Wang, G., Zhang, R., Duan, J., Gao, P., Lei, X., Qiu, H., Zhang, C., Zhang, Y. et al. (2022) Efficient targeted insertion of large DNA fragments without DNA donors. Nat Methods, 19, 331-340.
14. Ferreira da Silva, J., Oliveira, G. P., Arasa-Verge, E. A., Kagiou, C., Moretton, A., Timelthaler, G., Jiricny, J. and Loizou, J. I. (2022) Prime editing efficiency and fidelity are enhanced in the absence of mismatch repair. Nat Commun, 13, 760.
15. Schene, I. F., Joore, I. P., Oka, R., Mokry, M., van Vugt, A. H. M., van Boxtel, R., van der Doef, H. P. J., van der Laan, L. J. W., Verstegen, M. M. A., van Hasselt, P. M. et al. (2020) Prime editing for functional repair in patient-derived disease models. Nat Commun, 11, 5352.
16. Surun, D., Schneider, A., Mircetic, J., Neumann, K., Lansing, F., Paszkowski-Rogacz, M., Hanchen, V., Lee-Kirsch, M. A. and Buchholz, F. (2020) Efficient Generation and Correction of Mutations in Human iPS Cells Utilizing mRNAs of CRISPR Base Editors and Prime Editors. Genes (Basel), 11.
17. Adikusuma, F., Lushington, C., Arudkumar, J., Godahewa, G. I., Chey, Y. C. J., Gierus, L., Piltz, S., Geiger, A., Jain, Y., Reti, D. et al. (2021) Optimized nickase- and nuclease-based prime editing in human and mouse cells. Nucleic Acids Res, 49, 10785-10795.
18. Zhi, S., Chen, Y., Wu, G., Wen, J., Wu, J., Liu, Q., Li, Y., Kang, R., Hu, S., Wang, J. et al. (2022) Dual-AAV delivering split prime editor system for in vivo genome editing. Mol Ther, 30, 283-294.
19. Gao, Z., Ravendran, S., Mikkelsen, N. S., Haldrup, J., Cai, H., Ding, X., Paludan, S. R., Thomsen, M. K., Mikkelsen, J. G. and Bak, R. O. (2022) A truncated reverse transcriptase enhances prime editing by split AAV vectors. Mol Ther, 30, 2942-2951.
20. Zheng, C., Liang, S. Q., Liu, B., Liu, P., Kwan, S. Y., Wolfe, S. A. and Xue, W. (2022) A flexible split prime editor using truncated reverse transcriptase improves dual-AAV delivery in mouse liver. Mol Ther, 30, 1343-1351.
21. Petri, K., Zhang, W., Ma, J., Schmidts, A., Lee, H., Horng, J. E., Kim, D. Y., Kurt, I. C., Clement, K., Hsu, J. Y. et al. (2022) CRISPR prime editing with ribonucleoprotein complexes in zebrafish and primary human cells. Nat Biotechnol, 40, 189-193.
22. Li, H., Busquets, O., Verma, Y., Syed, K. M., Kutnowski, N., Pangilinan, G. R., Gilbert, L. A., Bateup, H. S., Rio, D. C., Hockemeyer, D. et al. (2022) Highly efficient generation of isogenic pluripotent stem cell models using prime editing. Elife, 11.
23. Kankia, B. I. and Marky, L. A. J. T. J. o. P. C. B. (1999) DNA, RNA, and DNA/RNA oligomer duplexes: A comparative study of their stability, heat, hydration, and Mg2+ binding properties. 103, 8759-8767.
24. Pulikkan, J. A., Hegde, M., Ahmad, H. M., Belaghzal, H., Illendula, A., Yu, J., O'Hagan, K., Ou, J., Muller-Tidow, C., Wolfe, S. A. et a!. (2018) CBFbeta-SMMHC Inhibition Triggers Apoptosis by Disrupting MYC Chromatin Dynamics in Acute Myeloid Leukemia. Cell, 174, 172-186 e121.
25. Zou, H., Jakovlic, I., Chen, R., Zhang, D., Zhang, J., Li, W. X. and Wang, G. T. (2017) The complete mitochondrial genome of parasitic nematode Camallanus cotti: extreme discontinuity in the rate of mitogenomic architecture evolution within the Chromadorea class. BMC Genomics, 18, 840.
26. Liu, B., Dong, X., Cheng, H., Zheng, C., Chen, Z., Rodriguez, T. C., Liang, S. Q., Xue, W. and Sontheimer, E. J. (2022) A split prime editor with untethered reverse transcriptase and circular RNA template. Nat Biotechnol, 40, 1388-1393.
27. Wu, Y., Zeng, J., Roscoe, B. P., Liu, P., Yao, Q., Lazzarotto, C. R., Clement, K., Cole, M. A., Luk, K., Baricordi, C. et al. (2019) Highly efficient therapeutic gene editing of human hematopoietic stem cells. Nat Med, 25, 776-783.
28. Clement, K., Rees, H., Canver, M. C., Gehrke, J. M., Farouni, R., Hsu, J. Y., Cole, M. A., Liu, D. R., Joung, J. K., Bauer, D. E. et al. (2019) CRISPResso2 provides accurate and rapid genome editing sequence analysis. Nat Biotechnol, 37, 224-226.
29. Kim, H. K., Yu, G., Park, J., Min, S., Lee, S., Yoon, S. and Kim, H. H. (2021) Predicting the efficiency of prime editing guide RNAs in human cells. Nat Biotechnol, 39, 198-206.
30. Philippe, C., Villard, L., De Roux, N., Raynaud, M., Bonnefond, J. P., Pasquier, L., Lesca, G., Mancini, J., Jonveaux, P., Moncla, A. et al. (2006) Spectrum and distribution of MECP2 mutations in 424 Rett syndrome patients: a molecular update. Eur J Med Genet, 49, 9-18.
31. Canver, M. C., Smith, E. C., Sher, F., Pinello, L., Sanjana, N. E., Shalem, O., Chen, D. D., Schupp, P. G., Vinjamur, D. S., Garcia, S. P. et al. (2015) BCL11A enhancer dissection by Cas9-mediated in situ saturating mutagenesis. Nature, 527, 192-197.
32. Sternberg, S. H., Redding, S., Jinek, M., Greene, E. C. and Doudna, J. A. (2014) DNA interrogation by the CRISPR RNA-guided endonuclease Cas9. Nature, 507, 62-67.
33. Trojan, J., Zeuzem, S., Randolph, A., Hemmerle, C., Brieger, A., Raedle, J., Plotz, G., Jiricny, J. and Marra, G. (2002) Functional analysis of hMLH1 variants and HNPCC-related mutations using a human expression system. Gastroenterology, 122, 211-219.
34. Peng, M., Xie, J., Ucher, A., Stavnezer, J. and Cantor, S. B. (2014) Crosstalk between BRCA-Fanconi anemia and mismatch repair pathways prevents MSH2-dependent aberrant DNA damage responses. EMBO J, 33, 1698-1712.
35. Dumousseau, M., Rodriguez, N., Juty, N. and Le Novere, N. (2012) MELTING, a flexible platform to predict the melting temperatures of nucleic acids. BMC Bioinformatics, 13, 101.
36. Woloszynek, J. R., Rothbaum, R. J., Rawls, A. S., Minx, P. J., Wilson, R. K., Mason, P. J., Bessler, M. and Link, D. C. (2004) Mutations of the SBDS gene are present in most patients with Shwachman-Diamond syndrome. Blood, 104, 3588-3590.
37. Boocock, G. R., Morrison, J. A., Popovic, M., Richards, N., Ellis, L., Durie, P. R. and Rommens, J. M. (2003) Mutations in SBDS are associated with Shwachman-Diamond syndrome. Nat Genet, 33, 97-101.
38. Nakashima, E., Mabuchi, A., Makita, Y., Masuno, M., Ohashi, H., Nishimura, G. and Ikegawa, S. (2004) Novel SBDS mutations caused by gene conversion in Japanese patients with Shwachman-Diamond syndrome. Hum Genet, 114, 345-348.
39. Vikkula, M., Boon, L. M., Carraway, K. L., 3rd, Calvert, J. T., Diamonti, A. J., Goumnerov, B., Pasyk, K. A., Marchuk, D. A., Warman, M. L., Cantley, L. C. et al. (1996) Vascular dysmorphogenesis caused by an activating mutation in the receptor tyrosine kinase TIE2. Cell, 87, 1181-1190.
40. Limaye, N., Wouters, V., Uebelhoer, M., Tuominen, M., Wirkkala, R., Mulliken, J. B., Eklund, L., Boon, L. M. and Vikkula, M. (2009) Somatic mutations in angiopoietin receptor gene TEK cause solitary and multiple sporadic venous malformations. Nat Genet, 41, 118-124.
41. Liu, R., Paxton, W. A., Choe, S., Ceradini, D., Martin, S. R., Horuk, R., MacDonald, M. E., Stuhlmann, H., Koup, R. A. and Landau, N. R. (1996) Homozygous defect in HIV-1 coreceptor accounts for resistance of some multiply-exposed individuals to HIV-1 infection. Cell, 86, 367-377.
42. Doyon, Y., Choi, V. M., Xia, D. F., Vo, T. D., Gregory, P. D. and Holmes, M. C. (2010) Transient cold shock enhances zinc-finger nuclease-mediated gene disruption. Nat Methods, 7, 459-460.
43. Roobol, A., Carden, M. J., Newsam, R. J. and Smales, C. M. (2009) Biochemical insights into the mechanisms central to the response of mammalian cells to cold stress and subsequent rewarming. FEBS J, 276, 286-302.
44. Maurissen, T. L. and Woltjen, K. (2020) Synergistic gene editing in human iPS cells via cell cycle and DNA repair modulation. Nat Commun, 11, 2876.

The publications (including patent publications), web sites, company names, books, manuals, treatise, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:
P1. A programmable prime editing system for modification of a double-stranded target DNA sequence comprising a target strand and a complementary non-target strand, the system comprising:
   a prime editor protein, the prime editor protein being a fusion protein comprising a nucleic acid programmable DNA binding domain fused to a reverse transcriptase domain, the DNA binding domain having nickase activity, and
   a pegRNA comprising, in a 5′ to 3′ direction:
      (i) a spacer sequence comprising a region of complementarity to the target strand of the double-stranded target DNA sequence;
      (ii) a gRNA core that interacts with the DNA binding domain; and
      (iii) an extension arm, the extension arm comprising, in a 5′ to 3′ orientation:
         (a) a DNA synthesis template encoding one or more nucleotide changes compared to a region downstream of a nick site in the non-target strand of the double-stranded target DNA sequence, and
         (b) a primer binding sequence comprising a region of complementarity to a primer sequence upstream of the nick site in the non-target strand of the double-stranded target DNA sequence and to a portion of the spacer sequence;
      wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.
P2. The programmable prime editing system of potential claim P1, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.
P3. The programmable prime-editing system according any one of the preceding potential claims, wherein the pegRNA comprises a modification conferring resistance to nuclease degradation.
P4. The programmable prime-editing system according any one of the preceding potential claims, wherein the pegRNA comprises at least one nucleotide comprising a modification conferring resistance to nuclease degradation.
P5. The programmable prime-editing system according any one of the preceding potential claims, wherein a 5′ series of 1-50 nucleotides of a 5′ terminus of the pegRNA comprises a modification conferring resistance to nuclease degradation.
P6. The programmable prime-editing system according any one of the preceding potential claims, wherein a 3′ series of 1-50 nucleotides of a 3′ terminus of the pegRNA comprises a modification conferring resistance to nuclease degradation.
P7. The programmable prime-editing system according any one of potential claims P5-P6, wherein, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.
P8. The programmable prime-editing system according any one of the preceding potential claims, wherein the primer binding sequence consists of 5-15 nucleotides.
P9. The programmable prime-editing system according any one of the preceding potential claims, wherein the primer binding sequence consists of 5-9 nucleotides.
P10. The programmable prime-editing system according any one of the preceding potential claims, wherein the primer binding sequence consists of a number of nucleotides, the number of nucleotides being selected so as to provide a primer binding sequence having a melting temperature (Tm) of 34°-40° C. with the primer sequence.
P11. The programmable prime-editing system according any one of the preceding potential claims, wherein the primer binding sequence consists of a number of nucleotides, the number of nucleotides being selected so as to provide a primer binding sequence having a melting temperature (Tm) of 35.5°-38.5° C. with the primer sequence.
P12. The programmable prime-editing system according any one of the preceding potential claims, wherein the programmable prime-editing system further comprises a competing oligonucleotide, the competing oligonucleotide being complementary to the primer binding sequence.
P13. The programmable prime-editing system of potential claim P12, wherein the competing oligonucleotide is further complementary to at least a portion of the DNA synthesis template.
P14. The programmable prime-editing system according any one of potential claims P12 and P13, wherein the competing oligonucleotide has a length of 5-25 nucleotides.
P15. The programmable prime-editing system according any one of potential claims P12-P14, wherein the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.
P16. The programmable prime editing system according to any one of the preceding potential claims, wherein the DNA synthesis template comprises at least one RTT self-avoiding base.
P17. A programmable prime editing system for modification of a double-stranded target DNA sequence comprising a target strand and a complementary non-target strand, the system comprising:
   a prime editor protein, the prime editor protein being a fusion protein comprising a nucleic acid programmable DNA binding domain fused to a reverse transcriptase domain, the DNA binding domain having nickase activity, and
   a pegRNA comprising, in a 5′ to 3′ direction:
      (i) a spacer sequence comprising a region of complementarity to the target strand of the double-stranded target DNA sequence;
      (ii) a gRNA core that interacts with the DNA binding domain; and
      (iii) an extension arm, the extension arm comprising, in a 5′ to 3′ orientation:
         (a) a DNA synthesis template encoding one or more nucleotide changes compared to a region downstream of a nick site in the non-target strand of the double-stranded target DNA sequence, and
         (b) a primer binding sequence comprising a region of complementarity to a primer sequence upstream of the nick site in the non-target strand of the double-stranded target DNA sequence and to a portion of the spacer sequence;
      wherein a terminus of the pegRNA selected from the group consisting of a 3′ terminus, a 5′ terminus, and combinations thereof, comprises a series of 1-50 nucleotides comprising a modification conferring resistance to nuclease degradation.
P18. The programmable prime editing system of potential claim P17, wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.
P19. The programmable prime editing system of potential claim P18, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.
P20. The programmable prime-editing system according any one of potential claims P17-P19, wherein, independently for any given nucleotide of the series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.
P21. The programmable prime-editing system according any one of potential claims P17-P20, wherein the primer binding sequence consists of 5-15 nucleotides.
P22. The programmable prime-editing system according any one of potential claims P17-P21, wherein the primer binding sequence consists of 5-9 nucleotides.
P23. The programmable prime-editing system according any one of potential claims P17-P22, wherein the primer binding sequence consists of a number of nucleotides, the number of nucleotides being selected so as to provide a primer binding sequence having a melting temperature (Tm) of 34°-40° C. with the primer sequence.
P24. The programmable prime-editing system according any one of potential claims P17-P23, wherein the primer binding sequence consists of a number of nucleotides, the number of nucleotides being selected so as to provide a primer binding sequence having a melting temperature (Tm) of 35.5°-38.5° C. with the primer sequence.
P25. The programmable prime-editing system according any one of potential claims P17-P24, wherein the programmable prime-editing system further comprises a competing oligonucleotide, the competing oligonucleotide being complementary to the primer binding sequence.
P26. The programmable prime-editing system of potential claim P25, wherein the competing oligonucleotide is further complementary to at least a portion of the DNA synthesis template.
P27. The programmable prime-editing system according any one of potential claims P25 and P26, wherein the competing oligonucleotide has a length of 5-25 nucleotides.
P28. The programmable prime-editing system according any one of potential claims P25-P27, wherein the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.
P29. The programmable prime editing system according to any one of potential claims P17-P28, wherein the DNA synthesis template comprises at least one RTT self-avoiding base.
P30. A programmable prime editing system for modification of a double-stranded target DNA sequence comprising a target strand and a complementary non-target strand, the system comprising:
   a prime editor protein, the prime editor protein being a fusion protein comprising a nucleic acid programmable DNA binding domain fused to a reverse transcriptase domain, the DNA binding domain having nickase activity, and
   a pegRNA comprising, in a 5′ to 3′ direction:
      (i) a spacer sequence comprising a region of complementarity to the target strand of the double-stranded target DNA sequence;
      (ii) a gRNA core that interacts with the DNA binding domain; and
      (iii) an extension arm, the extension arm comprising, in a 5′ to 3′ orientation:
         (a) a DNA synthesis template encoding one or more nucleotide changes compared to a region downstream of a nick site in the non-target strand of the double-stranded target DNA sequence,
         (b) a primer binding sequence comprising a region of complementarity to a primer sequence upstream of the nick site in the non-target strand of the double-stranded target DNA sequence and to a portion of the spacer sequence; and
   a competing oligonucleotide, the competing oligonucleotide being complementary to the primer binding sequence.
P31. The programmable prime editing system of potential claim P30, wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.
P32. The programmable prime editing system according to potential claim P31, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.
P33. The programmable prime-editing system according any one of potential claims P30-P32, wherein a 5′ series of 1-50 nucleotides of a 5′ terminus of the pegRNA comprises a modification conferring resistance to nuclease degradation.
P34. The programmable prime-editing system according any one of potential claims P30-P33, wherein a 3′ series of 1-50 nucleotides of a 3′ terminus of the pegRNA comprises a modification conferring resistance to nuclease degradation.
P35. The programmable prime-editing system according any one of potential claims P33 and P34, wherein, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.
P36. The programmable prime-editing system according any one of potential claims P30-P35, wherein the primer binding sequence consists of 5-15 nucleotides.
P37. The programmable prime-editing system according any one of potential claims P30-P36, wherein the primer binding sequence consists of 5-9 nucleotides.
P38. The programmable prime-editing system according any one of potential claims P30-P37, wherein the primer binding sequence consists of a number of nucleotides, the number of nucleotides being selected so as to provide a primer binding sequence having a melting temperature (Tm) of 34°-40° C. with the primer sequence.
P39. The programmable prime-editing system according any one of potential claims P30-P38, wherein the primer binding sequence consists of a number of nucleotides, the number of nucleotides being selected so as to provide a primer binding sequence having a melting temperature (Tm) of 35.5°-38.5° C. with the primer sequence.
P40. The programmable prime-editing system of any one of potential claims P30-P39, wherein the competing oligonucleotide is further complementary to at least a portion of the DNA synthesis template.
P41. The programmable prime-editing system according any one of potential claims P30-P40, wherein the competing oligonucleotide has a length of 5-25 nucleotides.
P42. The programmable prime-editing system according any one of potential claims P30-P41, wherein the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.
P43. The programmable prime editing system according to any one of potential claims P30-P42, wherein the DNA synthesis template comprises at least one RTT self-avoiding base.
P44. A programmable prime editing system for modification of a double-stranded target DNA sequence comprising a target strand and a complementary non-target strand, the system comprising:
   a prime editor protein, the prime editor protein being a fusion protein comprising a nucleic acid programmable DNA binding domain fused to a reverse transcriptase domain, the DNA binding domain having nickase activity, and
   a pegRNA comprising, in a 5′ to 3′ direction:
      (i) a spacer sequence comprising a region of complementarity to the target strand of the double-stranded target DNA sequence;
      (ii) a gRNA core that interacts with the DNA binding domain; and
      (iii) an extension arm, the extension arm comprising, in a 5′ to 3′ orientation:
         (a) a DNA synthesis template encoding one or more nucleotide changes compared to a region downstream of a nick site in the non-target strand of the double-stranded target DNA sequence,
         (b) a primer binding sequence comprising a region of complementarity to a primer sequence upstream of the nick site in the non-target strand of the double-stranded target DNA sequence and to a portion of the spacer sequence, and
         (c) a 3′ extension, the 3′ extension being complementary to the primer binding sequence, thereby forming a hairpin with the primer binding sequence.
P45. The programmable prime editing system of potential claim P44, wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.
P46. The programmable prime editing system according to potential claim P45, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.
P47. The programmable prime-editing system according any one of potential claims P44-P46, wherein a 5′ series of 1-50 nucleotides of a 5′ terminus of the pegRNA comprises a modification conferring resistance to nuclease degradation.
P48. The programmable prime-editing system according any one of potential claims P44-P47, wherein a 3′ series of 1-50 nucleotides of a 3′ terminus of the pegRNA comprises a modification conferring resistance to nuclease degradation.
P49. The programmable prime-editing system according any one of potential claims P47 and P48, wherein, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.
P50. The programmable prime-editing system according any one of potential claims P44-P49, wherein the primer binding sequence consists of 5-15 nucleotides.
P51. The programmable prime-editing system according any one of potential claims P44-P50, wherein the primer binding sequence consists of 5-9 nucleotides.
P52. The programmable prime-editing system according any one of potential claims P44-P51, wherein the primer binding sequence consists of a number of nucleotides, the number of nucleotides being selected so as to provide a primer binding sequence having a melting temperature (Tm) of 34°-40° C. with the primer sequence.
P53. The programmable prime-editing system according any one of potential claims P44-P52, wherein the primer binding sequence consists of a number of nucleotides, the number of nucleotides being selected so as to provide a primer binding sequence having a melting temperature (Tm) of 35.5°-38.5° C. with the primer sequence.
P54. The programmable prime-editing system of any one of potential claims P44-P53, wherein the 3′ extension is further complementary to at least a portion of the DNA synthesis template.
P55. The programmable prime-editing system according any one of potential claims P44-P54, wherein the 3′ extension is 5-25 nucleotides in length.
P56. The programmable prime-editing system according any one of potential claims P44-P55, wherein the 3′ extension consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.
P57. The programmable prime editing system according to any one of potential claims P44-P56, wherein the DNA synthesis template comprises at least one RTT self-avoiding base.
P58. A method for site-specific modification of a double-stranded target DNA sequence comprising a target strand and a non-target strand, the method comprising:
   contacting the double-stranded target DNA sequence with the programmable prime-editing system according to any one of the preceding potential claims,
   wherein the contacting results in:
      nicking the non-target strand of the double-stranded target DNA sequence to form a free 3′ end at the nick site;
      annealing the primer binding sequence with the primer sequence upstream of the nick site in the non-target strand of the double-stranded target DNA;
      synthesizing a single strand of DNA encoded by the DNA synthesis template from the free 3′ end of the non-target strand of the double-stranded target DNA sequence; and
      replacing the region downstream of the nick site in the non-target strand of the double-stranded target DNA sequence with the single strand of DNA encoded by the DNA synthesis template, thereby modifying the sequence of the double-stranded target DNA sequence.
P59. A non-naturally occurring pegRNA comprising, from 5′ to 3′:
   (i) a spacer sequence comprising a region of complementarity to a target strand of a double-stranded target DNA sequence;
   (ii) a gRNA core configured to interact with a DNA binding domain of a prime editor protein; and
   (iii) an extension arm, the extension arm comprising, in a 5′ to 3′ orientation:
      (a) a DNA synthesis template encoding one or more nucleotide changes compared to a region downstream of a nick site in a non-target strand of the double-stranded target DNA sequence, and
      (b) a primer binding sequence comprising a region of complementarity to a primer sequence upstream of the nick site in the non-target strand of the double-stranded target DNA sequence and to a portion of the spacer sequence;
   wherein the primer binding sequence comprises at least one self-avoiding base and the portion of the spacer sequence comprises at least one corresponding complement self-avoiding base so as to reduce base pairing of the primer binding sequence to the portion of the spacer sequence.
P60. The non-naturally occurring pegRNA of potential claim P59, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.
P61. The non-naturally occurring pegRNA of any one of potential claims P59 and P60, wherein a 5′ series of 1-50 nucleotides of a 5′ terminus of the pegRNA comprises a modification conferring resistance to nuclease degradation.
P62. The non-naturally occurring pegRNA of any one of potential claims P59-P61, wherein a 3′ series of 1-32 nucleotides of a 3′ terminus of the pegRNA comprises a modification conferring resistance to nuclease degradation.
P63. The non-naturally occurring pegRNA of any one of potential claims P61-P62, wherein, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.
P64. The non-naturally occurring pegRNA of any one of potential claims P59-P63, wherein the primer binding sequence consists of 5-15 nucleotides.
P65. The non-naturally occurring pegRNA of any one of potential claims P59-P64, wherein the primer binding sequence consists of 5-9 nucleotides.
P66. The non-naturally occurring pegRNA according any one of potential claims P59-P65, wherein the primer binding sequence consists of a number of nucleotides, the number of nucleotides being selected so as to provide a primer binding sequence having a melting temperature (Tm) of 34°-40° C. with the primer sequence.
P67. The non-naturally occurring pegRNA according any one of potential claims P59-P66, wherein the primer binding sequence consists of a number of nucleotides, the number of nucleotides being selected so as to provide a primer binding sequence having a melting temperature (Tm) of 35.5°-38.5° C. with the primer sequence.
P68. The non-naturally occurring pegRNA according to any one of potential claims P59-P67, wherein the DNA synthesis template comprises at least one RTT self-avoiding base.
P69. A non-naturally occurring pegRNA comprising, from 5′ to 3′:
   (i) a spacer sequence comprising a region of complementarity to a target strand of a double-stranded target DNA sequence;
   (ii) a gRNA core configured to interact with a DNA binding domain of a prime editor protein; and
   (iii) an extension arm, the extension arm comprising, in a 5′ to 3′ orientation:
      (a) a DNA synthesis template encoding one or more nucleotide changes compared to a region downstream of a nick site in a non-target strand of the double-stranded target DNA sequence, and
      (b) a primer binding sequence comprising a region of complementarity to a primer sequence upstream of the nick site in the non-target strand of the double-stranded target DNA sequence and to a portion of the spacer sequence,
      (c) a 3′ extension, the 3′ extension being complementary to the primer binding sequence, thereby forming a hairpin with the primer binding sequence.
P70. The non-naturally occurring pegRNA of potential claim P69, wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.
P71. The non-naturally occurring pegRNA of potential claim P70, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.
P72. The non-naturally occurring pegRNA of any one of potential claims P69-P71, wherein a 5′ series of 1-50 nucleotides of a 5′ terminus of the pegRNA comprises a modification conferring resistance to nuclease degradation.
P73. The non-naturally occurring pegRNA of any one of potential claims P69-P72, wherein a 3′ series of 1-32 nucleotides of a 3′ terminus of the pegRNA comprises a modification conferring resistance to nuclease degradation.
P74. The non-naturally occurring pegRNA of any one of potential claims P72-P73, wherein, independently for any given nucleotide of the 5′ series and the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.
P75. The non-naturally occurring pegRNA of any one of potential claims P69-P74, wherein the primer binding sequence consists of 5-15 nucleotides.
P76. The non-naturally occurring pegRNA of any one of potential claims P69-P75, wherein the primer binding sequence consists of 5-9 nucleotides.
P77. The non-naturally occurring pegRNA according any one of potential claims P69-P76, wherein the primer binding sequence consists of a number of nucleotides, the number of nucleotides being selected so as to provide a primer binding sequence having a melting temperature (Tm) of 34°-40° C. with the primer sequence.
P78. The non-naturally occurring pegRNA according any one of potential claims P69-P77, wherein the primer binding sequence consists of a number of nucleotides, the number of nucleotides being selected so as to provide a primer binding sequence having a melting temperature (Tm) of 35.5°-38.5° C. with the primer sequence.
P79. The non-naturally occurring pegRNA according any one of potential claims P69-P78, wherein the 3′ extension is further complementary to at least a portion of the DNA synthesis template.
P80. The non-naturally occurring pegRNA according any one of potential claims P69-P79, wherein the 3′ extension is 5-25 nucleotides in length.
P81. The non-naturally occurring pegRNA according any one of potential claims P69-P80, wherein the 3′ extension consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.
P82. The non-naturally occurring pegRNA according to any one of potential claims P69-P81, wherein the DNA synthesis template comprises at least one RTT self-avoiding base.
P83. A method of treating a subject having or suspected of having a disease or disorder, the method comprising administering the programmable prime-editing system according to any one potential claims 1-57, ex vivo, to a cell from the subject.
P84. The method of potential claim P83, wherein, following the administering of the system, the cell is incubated at 10°-34° C. for a period of time.
P85. The method according to potential claim P84, wherein the cell is incubated at 32°-42° C. for the period of time.
P86. The method according to any one of potential claims P84 and P85, wherein the cell is incubated at 34°-40° C. for the period of time.
P87. The method according to any one of potential claims P84-P86, wherein the cell is incubated at 35°-39° C. for the period of time.
P88. The method according to any one of potential claims P84-P87, wherein the cell is incubated at 35.5°-38.5° C. for the period of time.
P89. The method according to any one of potential claims P84-P88, wherein the cell is incubated for the period of time at a temperature, selected from the group consisting 20°±0.5° C., 21°±0.5° C., 22°±0.5° C., 23°±0.5° C., 24°±0.5° C., 25°±0.5° C., 26°±0.5° C., 27°±0.5° C., 28°±0.5° C., 29°:0.5° C., 30°±0.5° C., 31°±0.5° C., 32°±0.5° C., 33°±0.5° C., and 34°±0.5° C.
P90. The method according to any one of potential claims P84-P89, wherein the period of time is 1-96 hours.
P91. The method according to any one of potential claims P84-P90, wherein the period of time is 12-72 hours.
P92. The method according to any one of potential claims P84-P91, wherein the period of time is 24-72 hours.
P93. The method according to any one of potential claims P84-P92, wherein the period of time is 24-48 hours.

The embodiments of the invention described above are intended to be merely exemplary; numerous variations and modifications will be apparent to those skilled in the art. All such variations and modifications are intended to be within the scope of the present invention as defined in any appended claims.

1. A programmable prime editing system for modification of a double-stranded target DNA sequence comprising a target strand and a complementary non-target strand, the system comprising:
a prime editor protein, the prime editor protein being a fusion protein comprising a nucleic acid programmable DNA binding domain fused to a reverse transcriptase domain, the DNA binding domain having nickase activity, and
a pegRNA comprising, in a 5′ to 3′ direction:
(i) a spacer sequence comprising a region of complementarity to the target strand of the double-stranded target DNA sequence;
(ii) a gRNA core that interacts with the DNA binding domain; and
(iii) an extension arm, the extension arm comprising, in a 5′ to 3′ orientation:
(a) a DNA synthesis template encoding one or more nucleotide changes compared to a region downstream of a nick site in the non-target strand of the double-stranded target DNA sequence, and
(b) a primer binding sequence comprising a region of complementarity to a primer sequence upstream of the nick site in the non-target strand of the double-stranded target DNA sequence and to a portion of the spacer sequence;
wherein the primer binding sequence comprises at least one self-avoiding base and the portion of the spacer sequence comprises at least one corresponding complementary self-avoiding base so as to reduce base pairing of the primer binding sequence to the portion of the spacer sequence, thereby reducing auto-inhibition of the modification of the double-stranded target DNA sequence by the pegRNA.

2. The programmable prime editing system of claim 1, wherein a given one of the at least one self-avoiding base and a given one of the at least one corresponding complement self-avoiding base, respectively, is selected from the group consisting of 2-aminopurine and 2-thiouracil; 2-thiouracil and 2-aminopurine; hypoxanthine and N4-ethyl-cytosine; N4-ethyl-cytosine and hypoxanthine, N4-methyl-cytosine and hypoxanthine, and N4-methyl-cytosine and hypoxanthine.

3. The programmable prime-editing system of claim 1, wherein the pegRNA comprises a modification conferring resistance to nuclease degradation.

4. The programmable prime-editing system of claim 1, wherein the pegRNA comprises at least one nucleotide comprising a modification conferring resistance to nuclease degradation.

5. The programmable prime-editing system of claim 1, wherein a 5′ series of 1-50 nucleotides of a 5′ terminus of the pegRNA comprises a modification conferring resistance to nuclease degradation.

6. The programmable prime-editing system of claim 1, wherein a 3′ series of 1-50 nucleotides of a 3′ terminus of the pegRNA comprises a modification conferring resistance to nuclease degradation.

7. The programmable prime-editing system of claim 5, wherein, independently for any given nucleotide of the 5′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

8. The programmable prime-editing of claim 1, wherein the primer binding sequence consists of 5-15 nucleotides.

9. The programmable prime-editing system of claim 1, wherein the primer binding sequence consists of 5-9 nucleotides.

10. The programmable prime-editing system of claim 1, wherein the primer binding sequence consists of a number of nucleotides, the number of nucleotides being selected so as to provide a primer binding sequence having a melting temperature (Tm) of 34°-40° C. with the primer sequence.

11. The programmable prime-editing system of claim 1, wherein the primer binding sequence consists of a number of nucleotides, the number of nucleotides being selected so as to provide a primer binding sequence having a melting temperature (Tm) of 35.5°-38.5° C. with the primer sequence.

12. The programmable prime-editing system of claim 1, wherein the programmable prime-editing system further comprises a competing oligonucleotide, the competing oligonucleotide being complementary to the primer binding sequence.

13. The programmable prime-editing system of claim 12, wherein the competing oligonucleotide is further complementary to at least a portion of the DNA synthesis template.

14. The programmable prime-editing system of claim 12, wherein the competing oligonucleotide has a length of 5-25 nucleotides.

15. The programmable prime-editing system of claim 12, wherein the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.

16. The programmable prime editing system of claim 1, wherein the DNA synthesis template comprises at least one RTT self-avoiding base.

17-93. (canceled)

94. The programmable prime-editing system of claim 6, wherein, independently for any given nucleotide of the 3′ series, the modification conferring resistance to nuclease degradation is selected from the group consisting of (i) a phosphorothioate modification, (ii) a 2′ modification selected from the group consisting of a 2′ O-methyl modification, a 2′-fluoro modification, a 2′-O-methoxyethyl (2′-MOE) modification, a 2′-deoxy modification, and a 2′-amino modification, and (iii) the phosphorothioate modification and the 2′ modification.

95. The programmable prime-editing system of claim 13, wherein the competing oligonucleotide is further complementary to at least a portion of the DNA synthesis template.

96. The programmable prime-editing system of claim 13, wherein the competing oligonucleotide has a length of 5-25 nucleotides.

97. The programmable prime-editing system of claim 13, wherein the competing oligonucleotide consists of a number of nucleotides selected from the group consisting of 5, 6, 7, 8, 9, 10, 11, 12, 13, 14, 15, 16, 17, 18, 19, 20, 21, 22, 23, 24, and 25 nucleotides.


SEQUENCE LISTING (18 sequences)

SEQ ID NO: 1 - protein, 7 aa, synthetic construct
PKKKRKV

SEQ ID NO: 2 - protein, 30 aa, synthetic construct
MDSLLMNRRKFLYQFKNVRWAKGRRETYLC

SEQ ID NO: 3 - protein, 45 aa, synthetic construct
SGGSSGGSKRTAGSYPYDVPDYADGSEFESPKKKRKVSGGSSGGS

SEQ ID NO: 4 - DNA, 34 bp, synthetic construct
  source 1..34: mol_type = other DNA
agggcacctagaccgccaagctgaaggtgaccaa

SEQ ID NO: 5 - DNA, 34 bp, synthetic construct
  source 1..34: mol_type = other DNA
agggcacacagaccgccaagctgaaggtgaccaa

SEQ ID NO: 6 - DNA, 34 bp, synthetic construct
  source 1..34: mol_type = other DNA
cctcatggaatcccttctgcagcacctggatcgc

SEQ ID NO: 7 - DNA, 34 bp, synthetic construct
  source 1..34: mol_type = other DNA
cctcatggaatcccttctgcagcaccttgatcgc

SEQ ID NO: 8 - DNA, 34 bp, synthetic construct
  source 1..34: mol_type = other DNA
ctggaccctaatgattttgacttcatggtaactg

SEQ ID NO: 9 - DNA, 34 bp, synthetic construct
  source 1..34: mol_type = other DNA
ctggaccctaatgattttgacttcaccgtaactg

SEQ ID NO: 10 - DNA, 34 bp, synthetic construct
  source 1..34: mol_type = other DNA
ctggagcctgtgataaaagcaactgttagcttgc

SEQ ID NO: 11 - DNA, 31 bp, synthetic construct
  source 1..31: mol_type = other DNA
ctggagcctataaaagcaactgttagcttgc

SEQ ID NO: 12 - DNA, 34 bp, synthetic construct
  source 1..34: mol_type = other DNA
ccggtggcactgcggctggaggtgggggttaaag

SEQ ID NO: 13 - DNA, 34 bp, synthetic construct
  source 1..34: mol_type = other DNA
ccggtggcactgcggctggaggtgggtgttaaag

SEQ ID NO: 14 - DNA, 32 bp, synthetic construct
  source 1..32: mol_type = other DNA
accaaactgaaatctgtaagcaggcgggtaac

SEQ ID NO: 15 - DNA, 32 bp, synthetic construct
  source 1..32: mol_type = other DNA
accaaactgaaatctgtaagcaggtaagtaac

SEQ ID NO: 16 - DNA, 30 bp, synthetic construct
  source 1..30: mol_type = other DNA
gcgcgcatcaggaaggacgggctgaggatg

SEQ ID NO: 17 - DNA, 30 bp, synthetic construct
  source 1..30: mol_type = other DNA
gcgcgcatcaggaaggacgggttatggatg

SEQ ID NO: 18 - protein, 2188 aa, synthetic construct
MASMTGGQQMGRAAPAAKKKKLDGSVDKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTAGSYPYDVPDYADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSTGGGPGGGAAAGSGSPKKKRKVGSGSKRPAATKKAGQAKKKKLEHHHHHH